(19) United States
(12) Patent Application Publication
(10) Pub. No.: US 2025/0213531 A1
(43) Pub. Date: Jul. 3, 2025

(54) 5-MEO-DTM FOR THE TREATMENT OF BIPOLAR DISORDER

(71) Applicant: GH RESEARCH IRELAND LIMITED, Dublin 2 (IE)

(72) Inventors: Theis TERWEY, Berlin (DE); Conor BURKE, Laytown (IE); Naoise GAFFNEY, Dublin (IE)

(73) Assignee: GH RESEARCH IRELAND LIMITED, Dublin 2 (IE)

(21) Appl. No.: 18/851,336

(22) PCT Filed: Mar. 27, 2023
(86) PCT No.: PCT/EP2023/057845

§ 371 Date: Sep. 26, 2024

(30) Foreign Application Priority Data
Mar. 27, 2022 (EP) 22000084.8
Mar. 27, 2022 (EP) 22000085.5
Jan. 30, 2023 (EP) 23153996.6
Jan. 30, 2023 (EP) 23154014.7

(51) Int. Cl. A61K 31/4045; A61P 25/24

(52) U.S. Cl. CPC A61K 31/4045; A61P 25/24

(57) ABSTRACT

5-Methoxy-N,N-dimethyltryptamine (5-MeO-DMT) or a pharmaceutically acceptable salt thereof is used in treating a patient who is diagnosed with bipolar disorder, wherein the 5-MeO-DMT is administered via the intravenous, intramuscular or subcutaneous route.

### TECHNICAL FIELD

The present invention is directed to improved methods for the treatment of bipolar disorder (BD) comprising administering to a patient in need thereof a therapeutically effective amount of 5-methoxy-N,N-dimethyltryptamine (5-MeO-DMT) or of a pharmaceutically acceptable salt thereof. The treatment not only improves depressed mood, but in particular improves characteristic aspects of BD, such as sleep disturbance, psychomotor retardation (reduced energy and activity and reduced motivation), negative thinking (worthlessness; helplessness and hopelessness; guilt), anxiety, cognitive dysfunction (impaired concentration and memory) and social/emotional withdrawal or detachment (anhedonia, emotional withdrawal and affective flattening).

The treatment also counteracts suicidal ideation. Still further, the treatment improves mixed symptoms (psychotic symptoms; irritability; lability; increased motor drive; increased speech; agitation).

The treatment according to the invention reduces or eliminates the risk of treatment-emergent mania or hypomania.

### BACKGROUND OF THE INVENTION

Bipolar disorder is a mental health condition characterized by extreme mood swings that include emotional lows (major depressive episodes) and highs (manic or hypomanic episodes). Bipolar disorder is a recurrent chronic disorder that affects more than 1% of the world's population irrespective of ethnic origin or socioeconomic status.

Symptoms indicating a depressive episode include depressed mood, such as feeling sad, empty, hopeless or tearful; marked loss of interest or feeling no pleasure in all or almost all activities; significant weight loss when not dieting, weight gain, or decrease or increase in appetite; either insomnia or sleeping too much; either restlessness or slowed behaviour; fatigue or loss of energy; feelings of worthlessness or excessive or inappropriate guilt; decreased ability to think or concentrate, or indecisiveness; thinking about, planning or attempting suicide.

A major depressive episode generally includes five or more of these symptoms. It includes symptoms that are severe enough to cause noticeable difficulty in day-to-day situations, such as work, school, social activities or relationships.

During a manic or hypomanic episode, a patient behaves or feels abnormally energetic, happy or irritable, and the patient often makes impulsive decisions with little regard for the consequences. There is usually also a reduced need for sleep.

If the elevated mood is severe or associated with psychosis, it is called mania; if it is less severe, it is called hypomania. Hypomania does not involve psychotic symptoms.

In bipolar disorder, periods of depression and periods of abnormally elevated mood can each last from days to weeks. Episodes of mood swings may occur rarely or multiple times a year.

Bipolar disorder was previously called manic depression. However, it has long been recognised that the condition is different from major depressive disorder. A first formal separation of a distinct bipolar disorder (BD) with mania from nonbipolar major depressive disorder (MDD) was introduced by DSM-III (Diagnostic and Statistical Manual of Mental Disorders III; 1980).

BD is classified as bipolar I disorder if there has been at least one manic episode, with or without depressive episodes. It is classified as bipolar II disorder if there has been at least one hypomanic episode (but no full manic episodes) and one major depressive episode. If these symptoms are due to drugs or medical problems, they are not diagnosed as bipolar disorder.

Whereas hypomania is a milder form of mania, bipolar II disorder is not a milder form of bipolar I disorder. A 13.6-year study of the natural course of bipolar II disorder found that patients were symptomatic during 53.9% of follow-up weeks, with depressive symptoms present during 50.3% of follow-up weeks (Judd, L., Akiskal, H., Schettler, P., Coryell, W., Endicott, J., Maser, J., Solomon, D., Leon, A. and Keller, M., 2003. A Prospective Investigation of the Natural History of the Long-term Weekly Symptomatic Status of Bipolar II Disorder. Archives of General Psychiatry, 60 (3), p. 261). A similar study in patients suffering from bipolar I disorder found that patients were symptomatic during 47.3% of follow-up weeks, with depressive symptoms accounting for 31.9% of follow-up weeks (Judd, L., Akiskal, H., Schettler, P., Endicott, J., Maser, J., Solomon, D., Leon, A., Rice, J. and Keller, M., 2002. The Long-term Natural History of the Weekly Symptomatic Status of Bipolar I Disorder. Archives of General Psychiatry, 59 (6), p. 530). Thus, it is evident that depressive symptoms dominate the symptomatic status in both types of bipolar disorder-most notably so in bipolar II disorder. Evidence suggests that a greater proportion of patients suffering from bipolar II disorder attempt suicide (Karanti, A., Kardell, M., Joas, E., Runeson, B., Pålsson, E. and Landen, M., 2019. Characteristics of bipolar I and II disorder: A study of 8766 individuals. Bipolar Disorders, 22 (4), pp. 392-400).

In most cases, bipolar disorder is treated with medications and psychological counseling (psychotherapy). Current treatments are, however, associated with only limited success, for instance, because of the often limited or not durable treatment response, the late onset of response, side effects which limit the long-term drug administration, and inconvenient dosing regimens which often limit compliance of the patient.

The evidence for or against the use of antidepressants in BD patients remains highly inconsistent (Baldessarini 2020) and a recent nationwide study found that 54.9% of patients suffering from bipolar II disorder were being treated with an antidepressant drug (Karanti, Kardell et al. 2020). This is despite the fact that there are several reports that BD patients, when treated with antidepressants, can worsen clinically, displaying symptoms such as agitation, insomnia, anger and irritability with increased risk of suicidal behaviour (Baldessarini 2020). Treatment emergent mania or hypomania (TEM) is a significant risk associated with such treatments.

Although numerous treatment options beyond antidepressants are available for BD, many of these are associated with adverse effects. Quetiapine, which is approved for the acute treatment of depressive episodes in both bipolar I disorder and bipolar II disorder, is associated with weight-gain, dry mouth, sedation, somnolence, and dizziness—resulting in higher discontinuation rates due to adverse effects amongst patients receiving quetiapine in placebo-controlled trials (Calabrese, Keck et al. 2005, Thase, Macfadden et al. 2006, Suppes, Datto et al. 2010). Furthermore, quetiapine is usually titrated over a period spanning multiple days resulting in delayed onset of efficacy. Lurasidone has been shown to result in higher rates of nausea and extrapyramidal events compared to placebo (Loebel, Cucchiaro et al. 2014, Loebel, Cucchiaro et al. 2014). Moreover, olanzapine and olanzapine-fluoxetine combination may cause somnolence and weight gain (Tohen, Vieta et al. 2003).

Depression as the predominant psychopathology even in treated BD is associated not only with excess morbidity, but also mortality from co-occurring general-medical disorders. The risks for medical disorders including diabetes or metabolic syndrome, and cardiovascular disorders, and associated mortality rates are several-times above those for the general population or for patients with other psychiatric disorders.

Furthermore, the standardized mortality ratio for suicide with BD reaches 20-times above general-population rates and exceeds rates with other major psychiatric disorders.

The current status of BD treatment efficacy is appropriately summarized by Baldessarini et al:

“All available pharmacological treatments used for bipolar depression have limited efficacy and risk adverse metabolic or neurological effects.”

Thus, new treatments for BD are a major unmet medical need.

While there has recently been significant interest in hallucinogens for the treatment of mental disorders, this has so far not led to a treatment of BD. This is due to a general lack of relevant clinical data which would allow drawing conclusions on the clinical utility of hallucinogens in BD as well as due to specific concerns that hallucinogens may in fact lead to exacerbations of the disease, in particular by the induction of mania or hypomania.

Hallucinogens including psychedelics are chemical compounds, some naturally occurring, some synthetic, which are defined by their ability to induce in humans after consumption sensory distortions, such as changes in auditory and visual perception, as well as distortions of mood and cognition. The term hallucinogen encompasses a rather broad group of psychoactive molecules with different modes of action. Some mental disorders have been suggested as in principle being amenable for treatment with psychoactive molecules, like psychedelics.

However, no psychedelic drug has been approved by any regulatory agency. In fact, clinical experience with such molecules is still rather restricted.

One compound already investigated in clinical trials is 5-methoxy-N,N-dimethyltryptamine (5-MeO-DMT). WO 2020/169850 reports on tests in healthy volunteers as well as a clinical trial involving patients suffering from treatment resistant depression (TRD), i.e., a form of major depressive disorder.

Against this background, an aim of the invention is in particular the provision of therapies as well as dosing regimens and administration routes for such therapies which are more effective (i.e., a) larger percentage of patients experiencing a clinical response, b) a larger average clinical response, c) an earlier onset of the clinical response, and/or d) a more durable clinical response) than previously described therapies.

A further aim of the current invention is to provide a compound for improved psychoactive therapies as well as dosing regimens and administration routes for such therapies which have a better safety profile and/or are better tolerated than previously described therapies. Another aim of the current invention is to provide a compound for improved psychoactive therapies as well as dosing regimens and administration routes for such therapies which are more convenient than previously described therapies. Another aim of the current invention is to provide a compound for improved psychoactive therapies as well as dosing regimens and administration routes for such therapies which are associated with higher rates of patient compliance (including higher rates of treatment initiation) than previously described therapies. A still further aim of the current invention is to identify specific disease aspects and specific subgroups of disease aspects which benefit from such improved psychoactive therapies.

### SUMMARY OF THE INVENTION

The present invention relates to 5-methoxy-N, N-dimethyltryptamine (5-MeO-DMT) or a pharmaceutically acceptable salt thereof for use in treating a patient who is diagnosed with bipolar disorder.

The patient may be diagnosed with bipolar II disorder or with bipolar I disorder. Patients treated will typically suffer from a current major depressive episode. The patients may have been previously treated without success.

In the context of the present invention, 5-MeO-DMT or a pharmaceutically acceptable salt thereof is administered via intravenous, intramuscular or subcutaneous administration.

5-MeO-DMT or a pharmaceutically acceptable salt thereof is administered at a dose or in a dosage regimen that causes the patient to experience a peak psychedelic experience. A dosage of about 1 mg to about 10 mg 5-MeO-DMT or an equimolar amount of a pharmaceutically acceptable salt may be administered.

Success of the treatment may be assessed by various scales including, but not limited to, the Bipolar Depression Rating Scale (BDRS), the Montgomery-Åsberg Depression Rating Scale (MADRS) and the Clinical Global Impression-Severity scale (CGI-S). The treatment brings about various treatment effects.

A clinical response, as reflected, for instance, by a reduction in the Clinical Global Impression-Severity (CGI-S) score, generally occurs not later than about 2 hours after the last administration of 5-MeO-DMT or a pharmaceutically acceptable salt thereof. A clinical response is observed on day 1, for instance, after about 24 hours.

In case of the treatment of sleep disturbance, a clinical response as reflected, for instance, by a reduction in the CGI-S score, occurs not later than about 24 hours after the last administration of 5-MeO-DMT or a pharmaceutically acceptable salt thereof.

The clinical response preferably persists until at least 6 days; in particular until at least 14 days; more preferably until at least 28 days after the last administration of 5-MeO-DMT or a pharmaceutically acceptable salt thereof.

The patient does not experience treatment-emergent mania or hypomania.

The treatment in particular leads to an improvement in at least one of sleep disturbance, psychomotor retardation, negative thinking, anxiety, cognitive dysfunction and social/emotional withdrawal or detachment.

The treatment moreover reduces or eliminates suicidal ideation.

It also reduces or eliminates at least one of psychotic symptoms; irritability; lability; increased motor drive; increased speech; agitation.

### DETAILED DESCRIPTION OF THE INVENTION

### Definitions

As used in the context of the present invention, unless otherwise noted, the term “5-MeO-DMT” refers to the free base 5-MeO-DMT. It is contemplated that pharmaceutically acceptable salts of 5-MeO-DMT will typically also be used. Such salts are in particular acid addition salts, wherein the acid may be selected from, for instance, acetic acid, benzoic acid, citric acid, fumaric acid, hydrobromic acid, hydrochloric acid, hydrofluoric acid, hydroiodic acid, oxalic acid, succinic acid and triflic acid. A preferred example is the hydrobromide salt. The appropriate weight amount of a salt to be administered can be calculated from the weight amount of the free base, assuming that equimolar amounts are used.

As used in the context of the present invention, a “patient” to be treated is a human subject who is diagnosed with bipolar disorder, such as bipolar II disorder, by a licensed professional in accordance with accepted medical practice. Diagnosis can, for instance, be in accordance with the Diagnostic and Statistical Manual of Mental Disorders-Fifth Edition (DSM-5) published by the American Psychiatric Association. The diagnosis will be by a physician or a psychologist. It is not sufficient that the human subject himself considers that he is suffering from the disorder.

As used in the context of the present invention, “suicidal ideation” refers to thinking about, considering, or planning for suicide. The presence of suicidal ideation in a patient will be diagnosed by a physician or a psychologist, using established protocols and methods for diagnosing suicidality. It is generally not sufficient that the patient himself considers that he is suffering from suicidal ideation. In some situations, a patient experiencing suicidal ideation will be at imminent risk of committing suicide, or will be considered to have ‘intent to act.’

As used in the context of the present invention, unless otherwise noted, the terms “treating” and “treatment” shall include the management and care of a patient for the purpose of combating a disease, condition, or disorder and includes the administration of compounds and methods according to the present invention to alleviate the signs and/or symptoms of the disease or eliminate the disease, condition, or disorder.

As used in the context of the present invention, unless otherwise noted, the term “therapeutically effective amount” shall mean the amount of active compound or pharmaceutical ingredient that elicits the biological or clinical response in a human that is being sought by a researcher, medical doctor or other clinician, which includes alleviation of the signs and/or symptoms of the disease, condition or disorder being treated.

“Clinical response” includes, but is not limited to, improvements on rating scales. These scales assess various disease aspects. Scales which may be used according to the invention include the Brief Psychiatric Rating Scale (BPRS), the Bipolar Depression Rating Scale (BDRS), the Montgomery-Åsberg Depression Rating Scale (MADRS) and the 17-item Hamilton Depression Rating Scale (HAM-D). Further relevant scales to assess clinical outcome include the Young Mania Rating Scale (YMRS), the Clinician Administered Dissociative States Scale (CADSS), the Brief Psychiatric Rating Scale (BPRS) and the Columbia-Suicide Severity Rating Scale (C-SSRS).

A clinical response can also be assessed based on the Clinical Global Impression-Severity scale (CGI-S), the Patient Global Impression-Severity scale (PGI-S), the Clinical Global Impression-Improvement scale (CGI-I) or the Patient Global Impression-Improvement scale (PGI-I).

Individual items of the scales indicated as well as sub-combinations of individual items may be used to assess specific disease aspects.

When assessing a clinical response at an early timepoint after drug administration (e.g. at 2 hours) based on endpoints which have been developed for a longer recall period (e.g. normally 7 days for the MADRS), a rational modification of such endpoint (e.g. changing the MADRS recall period to 2 hours and carrying forward the sleep item recorded at baseline before drug administration) may be applied. The same applies with respect to the BDRS (in particular the item sleep disturbance) and any other scale applied herein, unless a recall period is specifically indicated.

The considerations outlined apply for early timepoints because, on the one hand, in order to assess a clinical response, the influence of the patient's status before the treatment on any score recorded after treatment should be kept as low as possible, whereas on the other hand the sleep item cannot be assessed 2 hours after drug administration.

At later timepoints, for instance, on day 1 or later, typically all items of the relevant scales to assess a clinical response can be assessed, using, if necessary, an adapted recall period, so that it is not necessary to carry forward any pre-treatment score.

The Pittsburgh Sleep Quality Index (PSQI) assesses overall sleep quality and disturbances. The PSQI is a self-rated questionnaire comprising 19 questions. Respondents are asked to indicate how frequently they have experienced certain sleep difficulties over the past month or another appropriate recall window.

The 19 self-rated questions assess a wide variety of factors relating to sleep quality, including estimates of sleep duration and latency and of the frequency and severity of specific sleep-related problems. These 19 items are grouped into seven component scores: (1) subjective sleep quality; (2) sleep latency; (3) sleep duration; (4) habitual sleep efficiency; (5) sleep disturbances; (6) use of sleeping medication; (7) daytime dysfunction.

Each component is assigned a score of 0 to 3. Higher scores indicate more acute sleep disturbances. Detailed Scoring Instructions for the Pittsburgh Sleep Quality Index can be found in the Appendix of Buysse et al. The Pittsburgh Sleep Quality Index: a new instrument for psychiatric practice and research. Psychiatry Res. 1989 May; 28 (2): 193-213.

The seven component scores are then summed to yield one global score, with a range of 0-21 points, “0” indicating no difficulty and “21” indicating severe difficulties in all areas. A global score cut-off of 5 distinguishes poor from good sleepers. A global score >5 indicates that a patient is having severe difficulties in at least two areas, or moderate difficulties in more than three areas.

If treatment outcome is assessed using the PSQI, treatment success is indicated (i) by a decrease of the score, preferably (ii) by a decrease to 5 or below.

The Verbal Recognition Memory (VRM) test assesses verbal memory and new learning. It measures the ability to encode and subsequently retrieve verbal information. During this task, 18 words are presented on screen and participants are subsequently asked to recall them in a free-recall stage. A recognition test is then carried out where the participant is shown 36 words (including target words and distractors) and is asked to answer ‘Yes’ or ‘No’ as to whether they saw the word previously. After a 20 minutes delay period, another recognition test is then carried out, this time with a new set of distractor words. Administration time is about 6 minutes (including immediate and delayed recall).

The Rapid Visual Information Processing (RVP) test is a sensitive tool for assessment of sustained attention. A white box is shown in the center of the screen, inside which single digits from 2 to 9 appear in a pseudo-random order at a rate of 100 digits per minute on screen. Patients must detect a series of target sequences (for example, 3-5-7, 2-4-6 and 4-6-8) and touch a button when they see the last digit of a target sequence. Nine target sequences appear every 100 numbers/every minute. Duration of task is about 7 minutes.

The Spatial Working Memory (SWM) Task requires retention and manipulation of visuospatial information. This self-ordered test provides a measure of strategy as well as working memory errors. The test involves a number of colored squares (boxes) shown on the screen which require a selection strategy to fill an empty column. The test takes about 4 minutes to complete. Outcome measures of the SWM include errors and strategy. The computerized Corsi Block will be the version of the SWM task used in this study.

The Digit Symbol Substitution Task (DSST) is a version of the original paper and pencil task taken from the Wechsler Adult Intelligence Scale (Royer, F. L., and Janowitch, L., 1973. Performance of process and reactive schizophrenics on a symbol-digit substitution task. Percept Mot Skills 37 (1): 63-70). The patient is shown an encoding scheme consisting of a row of squares at the top of the screen, wherein nine digits are randomly associated with particular symbols. The same symbols are presented in a fixed sequence at the bottom of the screen as a row of separate response buttons. The randomization procedure is chosen such that symbols never appear at the same ordinal position within both rows. The encoding scheme and the response buttons remain visible while the patient is shown successive presentations of a single digit at the center of the screen. The task is to match each digit with a symbol from the encoding list and click the corresponding response button. The number of digits correctly encoded within 3 minutes is the performance measure.

Treatment outcome is assessed by using one or more indices or scales at one or more time points after completion of a treatment course.

The assessment can be carried out after the acute psychedelic experience has subsided. An appropriate point in time for an early assessment is about 2 to 3 hours after the last administration. The assessment can be carried out, for instance, about 2 hours or about 3 hours after the last administration.

In case that more than one index or scale is administered this cannot be done simultaneously. Thus, while one index or scale may be administered about 2 hours after the last administration of 5-MeO-DMT, another one may be administered, for instance, about 3 hours after the last administration of 5-MeO-DMT. It is considered herein that assessments at both time points or generally within a time frame of about 2 to 3 hours equally reflect an early therapeutic outcome.

An assessment at day 1 or on day 1 means an assessment on the day following the administration. The assessment will be carried out not earlier than 12 hours after the last administration and in any event not earlier than one night after the last administration and not later than 36 hours after the last administration. The assessment can be carried out after about 24 hours.

An assessment at day 7 or on day 7 means an assessment on the seventh day following the administration (the day of administration is day 0). Analogous definitions apply for other assessment timings measured in days.

As used in the context of the present invention, unless otherwise noted, the term “administration” (or “application”) shall mean the introduction of an amount, which may be a predetermined amount, of active compound or pharmaceutical ingredient into a patient via any route. The active compound is administered by intravenous administration, by intramuscular administration or by subcutaneous administration.

As used in the context of the present invention, unless otherwise noted, the terms “dose” and “dosage” and “dosage amount” shall mean the amount of active compound or pharmaceutical ingredient which is administered to a patient in an individual administration. The term “dosage regimen” (or “dosing regimen”) shall mean a defined sequence of one or more individual administrations.

### Bipolar Disorder

Bipolar disorder is characterized by various symptoms and has various aspects.

The predominant psychopathology is depression, and the presentation of a patient experiencing a depressive phase may initially result in the diagnosis of that patient as having major depressive disorder (MDD). However, BD possesses multiple characteristics that define it as distinct from the latter even during the depressive phase.

Of particular interest here are the symptoms that are more strongly associated with BD compared to other psychiatric disorders, as these are the metrics against which patient treatment is assessed. Notwithstanding that many symptoms can be said to straddle multiple disorders, much work has been done to identify several symptoms that present strongly in BD patients: sleep disturbance, psychomotor retardation (reduced energy and activity and reduced motivation), negative thinking (feelings of worthlessness; helplessness and hopelessness; guilt), anxiety, cognitive dysfunction (impaired concentration and memory) and social/emotional withdrawal or detachment (anhedonia, emotional withdrawal and affective flattening). Characteristic symptoms further include suicidal ideation. Still further, characteristic symptoms include mixed symptoms (psychotic symptoms; irritability; lability; increased motor drive; increased speech; agitation).

Clinical assessment tools such as the Bipolar Depression Rating Scale (BDRS) have been developed and validated for use in BD, which take into account these symptoms.

The Bipolar Depression Rating Scale (BDRS) is designed to measure the severity of depressive symptoms in bipolar depression. The BDRS is validated for clinical use by trained raters. Based on a clinical interview, the BDRS items rate the severity of depressive and/or mixed symptoms expressed by patients currently and during the past few days. If there is a discordance between symptoms currently and the last few days, the rating should reflect current symptoms. The scale contains 20 questions and the maximum score possible is 60. Higher scores indicate greater severity.

The questions address depressed mood; sleep disturbance; appetite disturbance; reduced social engagement; reduced energy and activity; reduced motivation; impaired concentration and memory; anxiety; anhedonia; affective flattening; feelings of worthlessness; feelings of helplessness and hopelessness; suicidal ideation; feelings of guilt; psychotic symptoms; irritability; lability; increased motor drive; increased speech; agitation.

Each of these aspects is assessed and assigned a score of 0, 1, 2 or 3.

Depressed mood is scored as 0 if there is no self-reported and/or observed depression as evidenced by gloom, sadness, pessimism, hopelessness, and helplessness; 1 (mild) in case of brief or transient periods of depression, or mildly depressed mood; 2 (moderate) in case a depressed mood is clearly but not consistently present and other emotions are expressed, or depression is of moderate intensity; 3 (severe) in case of pervasive or continuous depressed mood of marked intensity.

Sleep disturbance (sleep dysregulation) is assessed based on the change in total amount of sleep over a 24-hour cycle, rated independent of the effect of external factors. It can either take the form of insomnia (reduction in total sleep time) or the form of hypersomnia (increase in total sleep time, inclusive of daytime sleep).

The rating for insomnia involves scores of 0 (no reduction in total sleep time); 1 (mild; reduction up to 2 hours); 2 (moderate; 2-4 hours); 3 (severe; more than 4 hours).

The alternative rating for hypersomnia involves scores of 0 (no increase in total sleep time, inclusive of daytime sleep); 1 (mild; less than 2 hours, or normal amount but nonrestorative); 2 (moderate; 2-4 hours); 3 (severe; greater than 4 hours).

Appetite disturbance is assessed based on the change in appetite and food consumption, rated independent of the effect of external factors. It can either take the form of loss of appetite or the form of increase in appetite.

The rating for loss of appetite involves scores of 0 (no change in appetite and food consumption); 1 (mild; no change in food intake, but has to push self to eat or reports that food has lost taste); 2 (moderate; some decrease in food intake); 3 (marked decrease in food intake, hardly eating).

The alternative rating for increase in appetite involves scores of 0 (no change in appetite and food consumption); 1 (mild; no change in food intake, but increased hunger); 2 (moderate; some increase in food intake, e.g., comfort eating); 3 (marked increase in food intake or cravings).

Reduced social engagement is scored as 0 if there are no subjective reports of reduced social and interpersonal engagement or interactions; 1 (mild) in case of slight reduction in social engagement with no impairment in social or interpersonal function; 2 (moderate) in case of clear reduction in social engagement with some functional sequelae, e.g., avoiding some social engagements or conversations; and 3 (severe) in case of marked reduction in social interaction or avoidance of almost all forms of social contact, e.g., refusing to answer the phone or see friends or family.

Reduced energy and activity is scored as 0 if there is no reduced energy, drive or goal directed behaviour; 1 (mild) in case of ability to engage in usual activities but with increased effort; 2 (moderate) in case of significant reduction in energy leading to reduction of some role-specific activities; and 3 (severe) in case of leaden paralysis or cessation of almost all role specific activities, (e.g., spending excessive time in bed, avoiding answering the phone, poor personal hygiene).

Reduced motivation is scored as 0 if there are no reports of subjective reduction in drive, motivation, and consequent goal directed activity; 1 (mild) in case of a slight reduction in motivation with no reduction in function; 2 (moderate) in case of a reduced motivation or drive with significantly reduced volitional activity or requiring substantial effort to maintain usual level of function; and 3 (severe) in case of reduced motivation or drive such that goal directed behaviour or function is markedly reduced.

Impaired concentration and memory are scored as 0 if there are no subjective reports of reduced attention, concentration, or memory, and consequent functional impairment; 1 (mild) in case of slight impairment of attention, concentration, or memory with no functional impairment; 2 (moderate) in case of significant impairment of attention, concentration, or forgetfulness with some functional impairment; 3 (severe) in case of marked impairment of concentration or memory with substantial functional impairment, e.g., unable to read or watch TV).

Anxiety is scored as 0 if there are no subjective reports of worry, tension, and/or somatic anxiety symptoms e.g., tremor, palpitations, dizziness, light-headedness, pins and needles, sweating, dyspnoea, butterflies in the stomach, or diarrhoea; 1 (mild; transient worry or tension about minor matters); 2 (moderate; significant anxiety, tension, or worry, or some accompanying somatic features); 3 (severe; marked continuous anxiety, tension, or worry that interferes with normal activity; or panic attacks).

Anhedonia is scored as 0 (no subjectively reduced ability to experience pleasure in usual activities); 1 (mild; slight reduction in pleasure from usually pleasurable activities); 2 (moderate; significant reduction in pleasure from usually pleasurable activities; some pleasure from isolated activities retained); or 3 (severe; complete inability to experience pleasure).

Affective flattening is scored as 0 if there is no subjective sense of reduced intensity or range of feelings or emotions; 1 (mild) in case of slight constriction of range of affect, or transient reduction in range or intensity of feelings; 2 (moderate) in case of significant constriction of range or intensity of feelings with preservation of some emotions, e.g., inability to cry; and 3 (severe) in case of marked and pervasive constriction of range of affect or inability to experience usual emotions.

Feelings of worthlessness (also simply referred to as worthlessness) are scored as 0 (no subjective sense, or thoughts, of decreased self-value or self-worth); 1 (mild; slight decrease in sense of self-worth); 2 (moderate; some thoughts of worthlessness and decreased self-worth) 3 (severe; marked, pervasive, or persistent feelings of worthlessness, e.g., feels others better off without them, unable to appreciate positive attributes).

Feelings of helplessness and hopelessness (also simply referred to as helplessness and hopelessness) characterize the subjective sense of pessimism or gloom regarding the future, inability to cope, or sense of loss of control. If this is absent, the score is 0. The score is 1 (mild) in case of occasional and mild feelings of not being able to cope as usual, or pessimism; it is 2 (moderate) in case the patient often feels unable to cope, or has significant feelings of helplessness or hopelessness which lift at times; it is 3 (severe) if there are marked and persistent feelings of pessimism, helplessness, or hopelessness.

Suicidal ideation relates to thoughts or feelings that life is not worthwhile; thoughts of death or suicide and is scored 0 if such thoughts are absent; 1 (mild) in case of thoughts that life is not worthwhile or is meaningless; 2 (moderate) in case of thoughts of dying or death, but with no active suicide thoughts or plans; 3 (severe) in case of thoughts or plans of suicide.

Feelings of guilt (also simply referred to as guilt) are scored as 0 if there is no subjective sense of self blame, failure, or remorse for real or imagined past errors; 1 (mild) in case of slight decrease in self-esteem or increased self-criticism; 2 (moderate) in case of significant thoughts of failure, self-criticism, inability to cope, or ruminations regarding past failures and the effect on others; able to recognise as excessive; 3 (severe) in case of marked, pervasive, or persistent guilt, e.g., feelings of deserving punishment; or does not clearly recognise as excessive.

Psychotic symptoms are scored as 0 if overvalued ideas, delusions, or hallucinations are absent; 1 (mild) in case of mild overvalued ideas, e.g., self-criticism or pessimism without clear effect on behaviour; 2 (moderate) in case of significant overvalued ideas with clear effect on behaviour, e.g., strong guilt feelings, clear thoughts that others would be better off without them; 3 (severe) in case of clear psychotic symptoms, e.g., delusions or hallucinations.

Irritability reports uncharacteristic subjective irritability, short fuse, easily angered, manifested by verbal or physical outbursts and is scored 0 if absent; 1 (mild) in case of slight subjective irritability which may not be overtly present; 2 (moderate) in case of verbal snappiness and irritability that is clearly observable in the interview; 3 (severe) in case of reports of physical outbursts, e.g., throwing/breaking objects, or markedly abusive verbal outbursts.

Lability is scored 0 if there are no observed mood lability or reported mood swings. It is scored 1 (mild) in case of subjective reports of mild increase in mood lability; 2 (moderate) if mood lability is clearly observable, moderate in intensity; 3 (severe) in case of marked and dominant mood lability, frequent or dramatic swings in mood.

Increase motor drive relates to subjective reports and objective evidence of increased motor drive and motor activity. It is scored 0 in case of normal motor drive: 1 (mild) in case of a slight increase in drive, not observable in the interview; 2 (moderate) in case of clear and observable increase in energy and drive; 3 (severe) if there is a marked or continuous increase in drive.

Increased speech relates to an observed increase in either the rate or quantity of speech, or observed flight of ideas. This item is scored 0 if such observations are absent; 1 (mild) if there is a slight increase in the rate or quantity of speech; 2 (moderate) in case of racing thoughts, or if the patient is significantly more talkative, clearly distractible, or in case of some circumstantiality; wherein this does not impede the interview; 3 (severe) in case of flight of ideas; which interferes with the interview.

Agitation is scored 0 if there is no observed restlessness or agitation; 1 (mild) in case of slight restlessness; 2 (moderate) in case of clear increase in level of agitation; 3 (severe) in case of marked agitation, e.g., near continuous pacing or wringing hands.

While a higher score on the BDRS scale indicates more severe disease, there are no generally accepted limits for when a patient is to be considered moderately or severely ill. BDRS score ranges used herein for indicating the severity of depressive episodes in patients with bipolar disorder are 13-18 for “mildly ill”, 19-23 for “moderately ill”, 24-36 for “markedly ill”, 37-39 for “severely ill”, and ·40 for “extremely ill”.

Various other scales are also useful to assess the severity of disease as well as the clinical outcome of treatments.

The CGI was developed to provide a brief, stand-alone assessment of the clinician's view of the patient's global functioning prior to and after a treatment (Busner, J. and Tagrum, S. D., 2007. The Clinical Global Impressions Scale: Applying a Research Tool in Clinical Practice. Psychiatry 2007, 29-37).

The CGI-Severity (CGI-S) is based on one question the clinician has to answer: “Considering your total clinical experience with this particular population, how mentally ill is the patient at this time?” This is rated on the following seven-point scale: 1=normal, not at all ill; 2=borderline mentally ill; 3=mildly ill; 4=moderately ill; 5=markedly ill; 6=severely ill; 7=among the most extremely ill patients.

The CGI-S can be used to assess treatment success by comparing scores before and after treatment.

Alternatively, treatment success can be assessed using the CGI-Improvement (CGI-I), which is similarly simple in its format. After the treatment, the clinician compares the patient's overall clinical condition to the one prior to the treatment (the so-called baseline value). Again, only one query is rated on a seven-point scale: “Compared to the patient's condition at admission to the project [prior to medication initiation], this patient's condition is: 1=very much improved since the initiation of treatment; 2=much improved; 3=minimally improved; 4=no change from baseline (the initiation of treatment); 5=minimally worse; 6=much worse; 7=very much worse since the initiation of treatment.”

The Patient Global Impression scale (PGI), also known as Subject Global Impression (SGI), is the counterpart to the Clinical Global Impressions scale (CGI). It consists of one item based on the CGI and adapted to the patient. It can measure disease severity (PGI-S) or disease improvement (PGI-I).

The Montgomery-Asberg Depression Rating Scale (MADRS) is a ten-item diagnostic questionnaire used to measure the severity of depressive episodes in patients with mood disorders (Montgomery, S. A., & Asberg, M. (1979). A new depression scale designed to be sensitive to change. The British Journal of Psychiatry 134, p. 382). It was designed as an adjunct to the Hamilton Rating Scale for Depression (HAM-D), which would be more sensitive to the changes brought on by antidepressants and other forms of treatment. Higher MADRS score indicates more severe depression. The items considered are apparent sadness; reported sadness; inner tension; reduced sleep; reduced appetite; concentration difficulties; lassitude; inability to feel; pessimistic thoughts; and suicidal thoughts, and each item yields a score of 0 to 6. The overall score ranges from 0 to 60. Score ranges used herein for assessing the severity of depressive episodes in patients with bipolar disorder are 13-18 for “mildly ill”, 19-23 for “moderately ill”, 24-36 for “markedly ill”, 37-39 for “severely ill”, and ·40 for “extremely ill” (Thase, 2021).

Using the structured interview guide for the MADRS (SIGMA) will increase the reliability of given scales (Williams, J. B. W and Kobak, K. A., 2008. Development and reliability of a structured interview guide for the Montgomery Asberg Depression Rating Scale (SIGMA). The British Journal of Psychiatry 192, p. 52; Freeman, M. P., Pooley, J., Flynn, M. J., Baer, L., Mischoulon, D., Mou, D. and Fava, M., 2017. Guarding the Gate. Remote Structured Assessments to Enhance Enrollment Precision in Depression Trials. Journal of Clinical Pharmacology 37 (2), p. 176).

The Hamilton Depression Rating Scale (Ham-D) is a clinician-administered depression assessment scale. The original version contains 17 items (HDRS 17) pertaining to symptoms of depression (Hamilton, M., 1960. A Rating Scale for Depression, J Neurol Neurosurg Psychiatry 23:56-62; Hamilton, M., 1967. Development of a rating scale for primary depressive illness. Br J Soc Clin Psychol 1967; 6 (4): 278-96). Although the scale was designed for completion after an unstructured clinical interview, there are now semi-structured interview guides available (Williams, J. B., 1988. A structured interview guide for the Hamilton Depression Rating Scale. Arch Gen Psychiatry 45 (8): 742-7). A later 21-item version includes 4 items intended to subtype the depression.

The Young Mania Rating Scale (YMRS; Young, R. C., Biggs, J. T., Ziegler, V. E., & Meyer, D. A. (1978). A rating scale for mania: reliability, validity and sensitivity. The British journal of psychiatry, 133 (5), 429-435) is one of the most frequently utilized rating scales to assess manic symptoms. The scale has 11 items and is based on the patient's subjective report of his or her clinical condition over the previous 48 hours. Additional information is based upon clinical observations made during the course of the clinical interview. The items are selected based upon published descriptions of the core symptoms of mania. The YMRS follows the style of the HAM-D with each item given a severity rating. There are four items that are graded on a 0 to 8 scale (irritability, speech, thought content, and disruptive/aggressive behaviour), while the remaining seven items are graded on a 0 to 4 scale. These four items are given twice the weight of the others to compensate for poor cooperation from severely ill patients. There are well described anchor points for each grade of severity. The authors encourage the use of whole or half point ratings once experience with the scale is acquired. The scale is generally done by a clinician or other trained rater with expertise with manic patients.

The Brief Psychiatric Rating Scale (BPRS) is intended to screen for psychiatric symptoms in a structured fashion. The scale is one of the most widely used scales to measure psychotic symptoms and was first published in 1962. The design has later been updated. The version most often used today includes 18 different areas for physicians or psychologists to evaluate (Overall, J. E. and Gorham, D. R., 1962. The brief psychiatric rating scale. Psychological Reports 10, p. 799; Overall, J. E. and Gorham, D. R., 1988. The Brief Psychiatric Rating Scale (BPRS): recent developments in ascertainment and scaling. Psychopharmacology Bulletin 22, p. 97).

The 18 items (somatic concern, anxiety, emotional withdrawal, conceptual disorganization, guilt feelings, tension, mannerisms and posturing, grandiosity, depressive mood, hostility, suspiciousness, hallucinatory behaviour, motor retardation, uncooperativeness, unusual thought content, blunted affect, excitement and disorientation) are scored and each item is rated on a scale of 1-7.

A physician or psychologist will complete two tasks during an approximate 15-minute interview with the patient:
They will ask the patient a series of questions from a list.
They will check if certain behaviours are displayed by the patient.

Based on the answers and the behaviours observed, a physician or psychologist will complete the BPRS form by ranking the severity of each area using a scale of one to seven: a score of one means an absence of signs or symptoms up to a score of seven that means it is present and at a severe level. If it is not possible to rate the specific signs or symptoms, a score of 0 or “Not assessed” is recorded.

The Clinician Administered Dissociative States Scale (CADSS) is rated by the investigator via an interview with the patient. The CADSS is a 27-item scale with 19 subject-rated items and 8 items scored by an observer. The rating ranges from 0 ‘not at all’ to 4 ‘extremely’ (Bremner, J. D., Krystal, J. H., Putnam, F. W., Southwick, S. M., Marmar, C., Charney, D. S., and Mazure, C. M., 1998. Measurement of Dissociative States with the Clinician-Administered Dissociative States Scale (CADSS). Journal of Traumatic Stress 11 (1), p. 125).

The CADSS is divided into 3 components: 1) depersonalization, 2) derealization and 3) amnesia. Summed together, these subscales form a total dissociative score. The CADSS is specifically designed to be a standardized measure of present-state dissociative symptomatology.

The Columbia Suicide Severity Rating Scale (C-SSRS) is a detailed questionnaire assessing both suicidal behaviour and suicidal ideation to help identify if there is an immediate need for medical intervention as well as providing data for the overall assessment of a treatment effect in relation to suicidality. The C-SSRS is evidence-supported and is part of a national and international public health initiative involving the assessment of suicidality (Posner, K., Brown, G. K., Stanley, B., Brent, D. A., Yershova, K. V., Oquendo, M. A., Currier, G. W., Melvin, G. A., Greenhill, L., Shen, S., and Mann, J. J., 2011. The Columbia-Suicide Severity Rating Scale: Initial Validity and Internal Consistency Findings From Three Multisite Studies With Adolescents and Adults. American Journal of Psychiatry 168 (12), p. 1266-77).

The questionnaire will be administered as an interview by a registered psychologist or physician.

The present invention provides for a treatment of a patient, as defined herein, diagnosed with bipolar disorder, especially bipolar II disorder, and in particular a patient diagnosed with bipolar disorder suffering from a current major depressive episode. The treatment in particular includes treatment of the above mentioned disease aspects, namely sleep disturbance, psychomotor retardation (reduced energy and activity and reduced motivation), negative thinking (worthlessness; helplessness and hopelessness; guilt), anxiety, cognitive dysfunction (impaired concentration and memory) and social/emotional withdrawal or detachment (anhedonia, emotional withdrawal and affective flattening).

The treatment also counteracts suicidal ideation. Still further, the treatment improves mixed symptoms (psychotic symptoms; irritability; lability; increased motor drive; increased speech; agitation).

The present invention in particular provides a treatment of depression in BD patients without inducing hypomania or mania.

### The Active Agent

The above discussion shows that BD is characterized by several aspects which as such present a significant disease burden and deserve appropriate treatment. Thus, there is not only a need for a treatment, in particular by pharmacological intervention, to improve overall disease scores but also to improve specific aspects of the disease.

The inventors considered that a carefully chosen hallucinogen may lead to an improved treatment of important aspects of BD and may lead to overall improvements of the disease.

One group of hallucinogens entails compounds which bind to the 5-hydroxytryptamine (5-HT) receptors, which are also referred to as serotonin receptors (described are 7 families 5-HT1 to 5-HT7 with several subtypes). Examples are lysergic acid diethylamide (LSD), psilocybin, and N,N-dimethyltryptamine (DMT). These serotonergic agents are often referred to as “psychedelics”, which emphasizes their predominant ability to induce qualitatively altered states of consciousness such as euphoria, trance, transcendence of time and space, spiritual experiences, dissolution of self-boundaries, or even near-death experiences, while other effects such as sedation, narcosis, or excessive stimulation are only minimal.

Chemically, serotonergic psychedelics are either phenylalkylamines or indoleamines, with the indoleamine class being divided into two subsets, ergolines and tryptamines, the latter being derived from tryptamine.

The various serotonergic psychedelics have different binding affinity and activation potency for various serotonin receptors, particularly 5-HT1A, 5-HT2A, and 5-HT2C, and their activity may also be modulated by interaction with other targets such as monoamine transporters and trace amine-associated receptors.

Recently published clinical studies which have used serotonergic psychedelic drugs such as LSD, psilocybin and DMT (using the shamanic brew Ayahuasca) in certain mental disorders suggest that those compounds could provide an alternative to the currently available treatments for certain mental disorders. However, there are reports that these compounds can induce mania in patients suffering from depressive symptoms, and this may preclude their use in the treatment of BD patients.

For instance, Lake et al. (Lake, C. R., Stirba, A. L., Kinneman, R. E. Jr, Carlson, B., Holloway, H. C., 1981. Mania associated with LSD ingestion. American Journal of Psychiatry. 138 (11): 1508-9) report about a patient who suffered a manic attack after ingesting LSD or an LSD analogue. The patient experienced acute symptoms of LSD intoxication, which resolved but were followed in about 3 weeks by a typical manic episode of psychotic magnitude. Hendin and Penn (Hendin, H. M., Penn, A. D., 2021. An episode of mania following self-reported ingestion of psilocybin mushrooms in a woman previously not diagnosed with bipolar disorder: A case report. Bipolar Disorders 23 (4): 1-3) report about an episode of mania following self-reported ingestion of psilocybin mushrooms. Szmulewicz et al. (Szmulewicz, A. G., Valerio, M. P., and Jose M Smith, J. M., 2015. Switch to mania after ayahuasca consumption in a man with bipolar disorder: a case report. International Journal of Bipolar Disorders (2015) 3:4) report on a switch to mania after consumption of ayahuasca, a DMT containing brew, in a man with bipolar disorder.

A further case report is found in Brown, T., Shao, W., Ayub, S., Chong, D., & Cornelius, C. (2017). A Physician's attempt to self-medicate bipolar depression with N, N-dimethyltryptamine (DMT). Journal of Psychoactive Drugs, 49 (4), 294-296.

The inventors considered that in order to avoid the induction of mania or hypomania or at least reduce the risk of induction of mania or hypomania, in particular in the treatment of BD patients, the compound administered must be appropriately chosen and preferably is administered in a particular dosing regimen.

The inventors identified 5-methoxy-N,N-dimethyltryptamine (5-MeO-DMT) as a psychedelic of particular interest for use in the therapy of bipolar disorder and its various aspects. 5-MeO-DMT has a distinct pharmacological profile which differs from that of other psychedelic compounds.

5-MeO-DMT is a potent, fast-acting, naturally occurring serotonin (5-HT) agonist, acting at both the 5-HT1A and the 5-HT2A receptor, with higher affinity for the 5-HT1A receptor subtype compared to other classical psychedelics.

Inhibition constants (Kᵢ values) as further detailed on the example section below for psilocin (the dephosphorylated from of psilocybin which is formed after uptake of psilocybin), DMT and 5-MeO-DMT are 48, 38 and 1.80 nM, respectively, at 5-HT1A receptors located in the hippocampus of post-mortem human brain. Thus, 5-MeO-DMT exhibits high affinity and psilocin and DMT exhibit moderate affinity for 5-HT1A receptors. Inhibition constants (Kᵢ values) for psilocin, DMT and 5-MeO-DMT are 37, 117 and 122 nM, respectively, at 5-HT2A receptors located in the frontal cortex of post-mortem human brain. Therefore, psilocin exhibits moderate/strong affinity and DMT and 5-MeO-DMT exhibit comparatively weak affinity for 5-HT2A receptors.

Relative to the other psychoactive compounds mentioned previously, 5-MeO-DMT displays an enhanced affinity for the 5-HT1A receptor, where it acts as a potent agonist. In the case of psilocin and DMT, there is an increased contribution of 5-HT2A binding, relative to 5-MeO-DMT, with the latter displaying the largest differential affinity for 5-HT1A over 5-HT2A of the three compounds. Therefore, 5-HT1A binding plays a much bigger role in the overall effect of 5-MeO-DMT relative to 5-HT2A binding compared to the other two compounds.

It has been reported that 5-HT1A agonism reduces impulsivity and aggression, whereas 5-HT2A agonism can result in short-term increases in these same traits. Furthermore, the dopamine system has been implicated in contributing to mania, with increased dopamine drive being linked to mania. LSD, psilocybin and DMT all display increased affinity for a variety of dopamine receptors relative to 5-MeO-DMT

Compared to other psychedelics, like LSD, psylocibin or DMT, 5-MeO-DMT can be administered to patients, preferably using dosing schemes as described herein, without a significant risk of inducing mania or hypomania in a patient suffering from a mental or nervous system disorder, including a disorder characterized by depressive episodes, for example, Major Depressive Disorder (MDD), Postpartum Depression (PPD), Persistent Depressive Disorder, Seasonal Affective Disorder and Bipolar Disorder (BD), such as Bipolar I Disorder and Bipolar II Disorder; a Psychotic Disorder, such as Schizophrenia;

or a personality disorder, such as Schizotypal Personality Disorder. The patient suffering from such a mental or nervous system disorder, treated according to the invention, does not experience treatment-emergent mania or hypomania.

It is also noted that reports of treatment-emergent mania or hypomania related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.” ·Journal of Psychiatric Practice·13.4 (2007): 233-237). However, the recently concluded clinical trial of 5-MeO-DMT in TRD patients showed no evidence of hypomania induction.

5-MeO-DMT can induce peak experiences, i.e., experiences characterized by an emotional perspective shift, which is described as “loss of ego” which often culminates in an overwhelming sense of “oneness with the universe”, more rapidly than other psychedelics and has a short duration of acute psychedelic effects (5 to 30 minutes after intravenous injection compared with several hours for e.g. oral psilocybin and oral LSD). These characteristics of 5-MeO-DMT are associated with an improved therapeutic profile which can be explained by specific alterations of Resting State Network (RSN) activity under 5-MeO-DMT treatment.

In particular, the Default Mode Network, which is one of several RSNs, has been implicated in a number of psychiatric disorders in which aberrant connectivity has been confirmed via functional MRI. This has been noted in bipolar disorder (Chai et al 2011, Wang et al 2016), albeit in conjunction with a number of distinguishing patterns of connectivity between several additional RSNs (Rai, 2021) and/or corticolimbic connectivity (e.g., between the prefrontal cortex and amygdala (de Almeida 2009)) that appear to differentiate bipolar from unipolar depression. While the involvement of the DMN and other RSNs in bipolar may be distinct from that of unipolar, the presence of aberrant connectivity combined with the improvements observed in BD-relevant symptoms and the absence of induced manic episodes in the recent clinical trial lets the inventors conclude that 5-MeO-DMT is suitable for the treatment of bipolar disorder, in particular if administered as described herein.

Various aspects of bipolar disorder, such as sleep disturbance, psychomotor retardation (reduced energy and activity and reduced motivation), negative thinking (worthlessness; helplessness and hopelessness; guilt), anxiety, cognitive dysfunction (impaired concentration and memory) and social/emotional withdrawal or detachment (anhedonia, emotional withdrawal and affective flattening) can be improved. Further aspects of the disease which can be improved include suicidal ideation and mixed symptoms (psychotic symptoms; irritability; lability; increased motor drive; increased speech; agitation). The improvements that can be achieved are reflected on clinically relevant scales.

Compared to other psychedelics, like LSD, psylocibin or DMT, 5-MeO-DMT can be administered to BD patients, using dosing schemes and administration routes as described herein, without a significant risk of inducing mania or hypomania.

Furthermore, 5-MeO-DMT is a 5-HT7 receptor agonist showing high affinity towards the receptor. The inventors determined, using recombinant human 5-HT7 receptor, [³H] LSD as a radio ligand and serotonin to estimate non-specific binding, a Kᵢ of 2.3 nM.

Thus, besides the 5-HT1A and 5-HT2A receptors discussed above, 5-MeO-DMT also interacts with the 5-HT7 receptor. 5-MeO-DMT act as an agonist on this receptor and shows a high (nanomolar) binding affinity.

The 5-HT7 receptor has a role in neurogenesis, synaptogenesis and dendritic spine formation. It is, among other things, associated with central processes such as learning and memory, with sleep regulation and circadian rhythm and with nociception.

The 5-HT7 receptor is in particular expressed in the spinal cord, raphe nuclei, thalamus, hypothalamus including the suprachiasmatic nucleus, hippocampus, prefrontal cortex, striatal complex, amygdala and in the Purkinje neurons of the cerebellum.

The suprachiasmatic nucleus is the central pacemaker of the circadian timing system. It coordinates circadian rhythms in various brain regions. Disruption of this coordination will result in disease states, in particular disease states involving sleep disturbance. In patients suffering from sleep disturbance resting state functional connectivity analysis reveals alterations in functional connectivity between the suprachiasmatic nucleus and regions within the default mode network.

The expression of the 5-HT7 receptor in the suprachiasmatic nucleus corresponds to the function of the receptor in regulation of sleep/wake cycles. The inventors consider that this allows treatment of patients suffering from sleep disturbance by 5-MeO-DMT which acts on the receptor.

The inventors consider that binding of 5-MeO-DMT to the 5-HT7 receptor as one mediator of the pharmacological effects of 5-MeO-DMT, which involve functional connectivity “resets” of networks and neuroplasticity effects, contributes to the beneficial effects of 5-MeO-DMT in the treatment of patients suffering from sleep disturbance.

The inventors further consider that binding of 5-MeO-DMT to the 5-HT7 receptor as well as to the 5-HT1A receptor as two mediators of effects exerted by 5-MeO-DMT, which include functional connectivity “resets” of networks and neuroplasticity effects, allows achieving beneficial effects also in patients suffering from other symptoms or conditions, such as cognitive dysfunction, anxiety, psychomotor retardation, negative thinking or social/emotional withdrawal. This is supported by the clinical results demonstrated in studies referred to herein.

Another feature of 5-MeO-DMT is its short half-life.

5-MeO-DMT is mainly inactivated through a deamination pathway mediated by monoamine oxidase A, and it is O-demethylated by cytochrome P450 2D6 (CYP2D6) enzyme.

The inventors investigated pharmacokinetic properties of 5-MeO-DMT and observed rapid absorption and distribution of inhaled 5-MeO-DMT, with maximum concentrations and pharmacological effects observed during and immediately after dosing.

An analysis of the pharmacokinetic properties of 5-MeO-DMT after inhalation shows a very rapid decline of the plasma concentration. Already 10 minutes after administration, the concentration drops to 10% of Cmax or below; after 2 hours, it is 1% of Cmax or below; after 3 hours, 5-MeO-DMT is no longer detectable in the plasma. This applies over the whole dose range tested (6 mg, 12 mg, 18 mg). No accumulation is observed upon repeated administration within a time frame of 1 to 4 hours. Uptitration as disclosed herein will not lead to accumulation and thus not to higher plasma concentrations, for instance, 10 minutes, 2 hours, or 3 hours after administration.

The properties of 5-MeO-DMT make the compound especially suitable for the treatment of BD, such as of bipolar II disorder, in particular for patients suffering from a current major depressive episode.

The properties of 5-MeO-DMT also allow specific dosage regimens, as discussed in more detail below.

According to the invention, isotopic variants of 5-MeO-DMT and pharmaceutically acceptable salts thereof can also be used. When reference is made to the use of 5-MeO-DMT or a pharmaceutically acceptable salt thereof, the use of isotopic variants is also contemplated.

These variants are in particular deuterated forms of 5-MeO-DMT and pharmaceutically acceptable salts of such forms.

Deuterated forms of 5-MeO-DMT are forms having a higher deuterium content than expected based on the natural abundance of this isotope.

Deuterated forms of 5-MeO-DMT are in particular forms wherein deuterium has been introduced at one or more defined hydrogen positions.

Examples of deuterated forms of 5-MeO-DMT include, without limitation, 1-deuterio-2-(5-methoxy-1H-indol-3-yl)-N,N-dimethylethanamine, 1,1-dideuterio-2-(5-methoxy-1H-indol-3-yl)-N,N-dimethylethanamine, 1,1,2,2-tetradeuterio-2-(5-methoxy-1H-indol-3-yl)-N,N-dimethylethanamine, and N,N-dimethyl-2-[5-(trideuteriomethoxy)-1H-indol-3-yl]ethanamine.

Further examples include forms of 5-MeO-DMT wherein deuterium has been introduced at one or more hydrogen positions of the N-bound methyl groups. Still further examples include forms of 5-MeO-DMT wherein one or more deuterium atoms replace hydrogen atoms of the indole ring system. It is moreover noted that combinations of the above substitution patterns are also contemplated.

Preparation methods for these compounds are known in the art.

According to the invention, mixtures of deuterated forms of 5-MeO-DMT, mixtures of one or more deuterated form with non-deuterated 5-MeO-DMT, pharmaceutically acceptable salts of deuterated forms of 5-MeO-DMT, mixture of such salts as well as mixtures of salts of deuterated and non-deuterated 5-MeO-DMT can also be used.

Further according to the invention, deuterated 5-MeO-DMT and salts of deuterated 5-MeO-DMT are used in amounts that are equimolar to the amounts of the corresponding non-deuterated forms.

According to the invention, prodrugs of 5-MeO-DMT and pharmaceutically acceptable salts of such prodrugs can also be used. Such prodrugs of 5-MeO-DMT can be metabolically converted to 5-MeO-DMT. Thus, when reference is made to the use of 5-MeO-DMT or a pharmaceutically acceptable salt thereof, his can be replaced by a 5-MeO-DMT prodrug or a salt thereof.

In suitable prodrugs, the hydrogen in position 1 of the indole moiety is substituted by an organic moiety which can be split off after administration.

Examples of suitable organic moieties are —C(O)OR¹, —C(O)R², —CH(R³)OR⁴, —C(O)OCH(R³)OC(O)R⁴, —C(O)OCH(R³)OC(O)OR⁴, —CH(R³)C(O)R⁴, —CH(R³)OC(O)R⁴, —CH(R³)OC(O)OR⁴, wherein each of R¹, R², R³, and R⁴ is independently hydrogen, alkyl, heteroalkyl, cycloalkyl, heterocyclylalkyl, aryl, or heteroaryl, wherein each alkyl, heteroalkyl, cycloalkyl, heterocyclylalkyl, aryl, and heteroaryl is independently substituted or unsubstituted.

Preferred examples of organic moieties are —CH(R³)OC(O)R⁴ and —C(O)OR¹, wherein R¹, R³, and R⁴ are defined as above.

Prodrugs, especially those of the above structure, can also be used on the form of pharmaceutically acceptable salts.

Specific examples of prodrugs are 5-MeO-DMT carboxy-isopropyl valinate, preferably in salt form, in particular as ditrifluoroacetate (1-(((S)-2-amino-3-methylbutanoyl)oxy)-2-methylpropyl 3-(2-(dimethylamino)ethyl)-5-methoxy-1H-indole-1-carboxylate di-trifluoroacetate) and 5-MeO-DMT methyl pivalate (3-(2-(dimethylamino)ethyl)-5-methoxy-1H-indol-1-yl)methyl pivalate).

Preparation methods for prodrugs as discussed herein are known in the art.

According to the invention, the Tₘₐₓ value of the metabolite 5-MeO-DMT as measured in male Sprague-Dawley (SD) rats following oral dosing of the prodrug at 10 mg/kg is preferably 1 hour or less, more preferably 0.7 hours or less and in particular 0.5 hours or less.

Further according to the invention, prodrugs of 5-MeO-DMT and salts of prodrugs of 5-MeO-DMT are used in amounts that are equimolar to the amounts of the corresponding non-prodrug forms.

### Patients

A patient treated according to the invention is diagnosed with bipolar disorder by a licensed professional in accordance with accepted medical practice. Diagnosis can, for instance, be in accordance with the Diagnostic and Statistical Manual of Mental Disorders-Fifth Edition (DSM-5) published by the American Psychiatric Association.

In one aspect, the patient is diagnosed with bipolar II disorder. In another aspect, the patient is diagnosed with bipolar I disorder.

Typically, the patient whether diagnosed with bipolar II disorder or with bipolar I disorder, suffers from a current major depressive episode.

The severity of a current major depressive episode may be assessed using the Montgomery-Åsberg Depression Rating Scale (MADRS). The patient may have a total score of equal to or greater than 19, such as greater or equal than 24, in particular greater or equal than 37.

Alternatively or in addition, the patient may have a Bipolar Depression Rating Scale (BDRS) total score of 19, such as greater or equal than 24, in particular greater or equal than 37.

Further alternatively or in addition, the patient may have a Hamilton Depression Rating Scale (HAM-D) total score of 19; such as greater or equal than 24; in particular greater or equal than 37.

The patient may suffer from treatment resistant disease. Treatment resistance means that the patient had no adequate improvement after at least two adequate courses of therapy. The patient in particular had no adequate improvement after at least two adequate courses of therapy, wherein at least one of the two courses was a pharmacotherapy; for instance, the patient had no adequate improvement after at least two adequate courses of pharmacotherapy. The at least two prior courses of treatment were in particular administered in the current episode of depression.

A patient with a major depressive episode treated according to the invention will usually have a Young Mania Rating Scale (YMRS) total score less than or equal to 8.

### Treatment of Aspects of BD

As indicated above, the treatment according to the invention addresses various aspects of bipolar disorder.

These include sleep disturbance, psychomotor retardation (reduced energy and activity and reduced motivation), negative thinking (worthlessness; helplessness and hopelessness; guilt), anxiety, cognitive dysfunction (impaired concentration and memory) and social/emotional withdrawal or detachment (anhedonia, emotional withdrawal and affective flattening).

The treatment also counteracts suicidal ideation. Still further, the treatment improves mixed symptoms (psychotic symptoms; irritability; lability; increased motor drive; increased speech; agitation).

Treatment according to the invention of a patient suffering from bipolar disorder will typically address more than one of the aspects listed above. Treatment will typically lead to a clinical response in several or all of the above aspects and to concomitant overall improvements.

According to the invention the above aspects can also be treated if they occur independent of bipolar disorder, for instance, in the context of a different mental disease. A clinical response can be achieved independent of whether or not the patient is diagnosed with bipolar disorder.

A treatment according to the invention reduces or eliminates (or improves or eliminates) an aspect of the diseases. As used herein this means that, if the aspect is assessed on the BDRS scale, there is an improvement by at least one point (reduction) or that the patient is in complete remission after the treatment (elimination), i.e., the respective aspect is scored 0.

If the aspect is assessed on the MADRS scale, there is an improvement by at least one point (reduction) or the patient is in complete remission after the treatment (elimination), i.e., the respective aspect is scored 0.

If the aspect is assessed on the BPRS scale, there is an improvement by at least one point (reduction) or the patient is in complete remission after the treatment (elimination), i.e., the respective aspect is scored 1.

A clinical response may also be reflected by a reduction in the Clinical Global Impression-Severity (CGI-S) score. According to the invention, a reduction in the CGI-S score means that the CGI-S is reduced by at least 1. Preferably, the CGI-S is reduced by at least 2 and/or to a score of 0. It is especially preferred if the CGI-S is reduced by at least 3 and/or to a score of 0.

To further support the clinical application of 5-MeO-DMT in patients suffering from BD the inventors assessed clinical data relating to the use of 5-MeO-DMT in patients treated because of mental disease and noted particular improvements in disease aspects typically also observed in patients with bipolar disorder. The inventors in particular noted improvements in various symptoms and combinations of symptoms which are characteristic of BD.

The data stem from a recently completed clinical trial investigating the use of 5-MeO-DMT in the treatment of patients diagnosed with Treatment Resistant Depression (TRD; see also the examples section below). While TRD is a condition different from BD, the inventors determined, as discussed in detail below, that certain clinical observations are made in the trial are relevant for devising a treatment for BD.

In the clinical trial, 5-MeO-DMT was administered via inhalation (as described in more detail in the example section below). Patients were assigned to different groups. In the context of the present invention, the group who received a single, 12 mg dose and the group who underwent an intra-day individualized dosing regimen (IDR) that allowed for multiple, escalating doses (6 mg, 12 mg and 18 mg) within a single day, driven by the intensity of the patient-reported psychedelic experience are of interest.

The data gathered include the assessment of the treated patients against several scales including the Montgomery-Åsberg Depression Rating Scale (MADRS) and the Brief Psychiatric Rating Scale (BPRS). While the focus of the trial was on demonstrating treatment efficacy through improvements in overall MADRS score, the inventors focused on the items comprising the various rating scales and noticed an overlap between several of these subscore items and the symptoms outlined above as being particularly noteworthy in BD patients. Multiple patients within the recruited cohort displayed significant improvements in one or more of these subscore items, a result that confirms the inventors' finding that 5-MeO-DMT is a compound suitable for treating BD patients, in particular BD patients presenting with these symptoms.

The specific subscore items in each of the scales are identified in more detail below. While these items do not always provide a verbatim match with the BD-related symptoms outlined above, the inventors recognised the overlap that exists between these items and those symptoms and concluded that the improvements noted against these items will translate into improvements against the related symptoms that have been included explicitly within BD-specific assessment scales, such as the BDRS. Indeed, given the degree of support within the literature for the association of these symptoms with BD, the inventors conclude that efficacy in treating one or more of these symptoms will result in significant improvements in overall outcomes in BD patients treated using 5-MeO-DMT.

One aspect of bipolar disorder, in particular bipolar II disorder, which can be treated by administration of 5-MeO-DMT, is sleep disturbance. 5-MeO-DMT can be administered to BD patients to improve the quality of sleep in said patients.

Sleep disturbance including variability in total sleep time (insomnia/hypersomnia) and circadian rhythm abnormalities have in particular been noted in patients suffering from bipolar disorder, as described by Kaplan et al, Gottlieb et al and others.

It has further been suggested that there is an association of the Default Mode Network (DMN) with sleep disruption (De Havas et al; Nie et al). The inventors consider that the association of the DMN with sleep disruption and the influence of 5-MeO-DMT, upon appropriate administration and dosing, on the DMN indicates that sleep dysregulation can be treated with 5-MeO-DMT.

In the above mentioned clinical studies involving the administration of 5-MeO-DMT, among others the MADRS item “reduced sleep”, which reflects insomnia, was assessed.

The MADRS item “reduced sleep” represents the experience of reduced duration or depth of sleep compared to the subject's own normal pattern when well. A score of 0 is assigned when the subject sleeps as usual. A score of 2 reflects slight difficulty dropping off to sleep or slightly reduced, light or fitful sleep. A score of 4 means that sleep is reduced or broken by at least two hours. A score of 6 means less than two or three hours sleep.

In the study group receiving the individualized dosing regimen, the aggregated score for the MADRS item “reduced sleep” across all 8 patients was 25 at base line. At day 1 after treatment, the earliest timepoint for assessing an impact of the treatment on sleep, it was reduced to 12 which corresponds to an improvement of 13 points or 52%. At day 7 after treatment, it was reduced to 9 which corresponds to an improvement of 16 points or 64%.

In the 12 mg group, the aggregated score for the MADRS item “reduced sleep” across all 4 patients was 12 at base line. At day 1 after treatment, it was reduced to 10 which corresponds to an improvement of 2 points or 17%. At day 7 after treatment, it was reduced to 6 which corresponds to an improvement of 6 points or 50%.

Thus, the score of the scale item that is of particular relevance to sleep disturbance, “reduced sleep”, is markedly improved. The inventors conclude that 5-MeO-DMT can be used to treat sleep disturbance in patients, in particular patients suffering from mental illness, such as BD.

Consequently, according to the invention, the treatment of a patient suffering from sleep disturbance with 5-MeO-DMT or a pharmaceutically acceptable salt thereof reduces or eliminates the sleep disturbance.

The reduction or elimination of sleep disturbance may be reflected by an improvement at least in the score of the BDRS item sleep disturbance on day 1, for instance, about 24 hours; on day 7; on day 14; and/or on day 28 after the last administration of 5-MeO-DMT or a pharmaceutically acceptable salt thereof.

The reduction or elimination of sleep disturbance as reflected by an improvement in the score of the BDRS item sleep disturbance occurs not later than about 24 hours after the last administration of 5-MeO-DMT or a pharmaceutically acceptable salt thereof. The reduction or elimination of sleep disturbance as reflected by an improvement in the score of the BDRS item sleep disturbance preferably persists until at least 6 days; in particular until at least 14 days; more preferably until at least 28 days after the last administration of 5-MeO-DMT or a pharmaceutically acceptable salt thereof.

If the sleep disturbance is reduced sleep, the reduction or elimination of sleep disturbance may be reflected by at least an improvement in the score of the MADRS item reduced sleep on day 1, for instance, about 24 hours; on day 7; on day 14; and/or on day 28 after the last administration of 5-MeO-DMT or a pharmaceutically acceptable salt thereof.

The reduction or elimination of sleep disturbance as reflected by an improvement in the score of the MADRS item reduced sleep occurs not later than about 24 hours after the last administration of 5-MeO-DMT or a pharmaceutically acceptable salt thereof. The reduction or elimination of sleep disturbance as reflected by an improvement in the score of the MADRS item reduced sleep preferably persists until at least 6 days; in particular until at least 14 days; more preferably until at least 28 days after the last administration of 5-MeO-DMT or a pharmaceutically acceptable salt thereof.

If the patient suffers from sleep disturbance the improvement in sleep disturbance is reflected by a reduction in the Clinical Global Impression-Severity (CGI-S) score on day 1, for instance, after about 24 hours; on day 7; on day 14 and/or on day 28 after the last administration of 5-MeO-DMT or a pharmaceutically acceptable salt thereof.

An improvement in sleep disturbance as reflected by a reduction in the Clinical Global Impression-Severity (CGI-S) score, occurs not later than about 24 hours after the last administration of 5-MeO-DMT or a pharmaceutically acceptable salt thereof.

An improvement in sleep disturbance, as reflected by at least a score of “much improved” in the Clinical Global Impression-Improvement (CGI-I) score or the Patient Global Impression-Improvement (PGI-I) score, preferably occurs not later than about 24 hours after the last administration of 5-MeO-DMT or a pharmaceutically acceptable salt thereof.

An improvement in sleep disturbance, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, sleep disturbance is an item of the BDRS. Since sleep disturbance furthermore also affects other aspects of BD, the inventors conclude that the observed improvement in the score of the “reduced sleep” item on the MADRS will yield not only a correlated improvement in the score of the “sleep disturbance” BDRS scale item, but additionally contribute to an overall improvement of the BDRS score.

If the patient suffers from sleep disturbance the reduction or elimination of sleep disturbance is reflected by an improvement in the Pittsburgh Sleep Quality Index (PSQI) global score on day 1, for instance, after about 24 hours; on day 7; on day 14 and/or on day 28 after the last administration of 5-MeO-DMT or a pharmaceutically acceptable salt thereof, wherein the recall period spans from the time point when acute psychedelic experiences have subsided after the last administration to the assessment time point.

If the patient suffers from sleep disturbance the reduction or elimination of sleep disturbance as reflected by an improvement in the Pittsburgh Sleep Quality Index (PSQI) global score occurs not later than about 24 hours after the last administration of 5-MeO-DMT or a pharmaceutically acceptable salt thereof, wherein the recall period spans from the time point when acute psychedelic experiences have subsided after the last administration to the assessment time point. The reduction or elimination of sleep disturbance as reflected by an improvement in the score of the PSQI preferably persists until at least 6 days; in particular until at least 14 days; more preferably until at least 28 days after the last administration of 5-MeO-DMT or a pharmaceutically acceptable salt thereof.

Another aspect of bipolar disorder, in particular bipolar II disorder, which can be treated by administration of 5-MeO-DMT, is psychomotor retardation, i.e., a combination of reduced energy and activity and reduced motivation. Psychomotor retardation involves a slowing down of thought and a reduction of physical movements in an individual. Psychomotor impairment can cause a visible slowing of physical and emotional reactions. Psychomotor retardation has been noted in patients suffering from bipolar disorder. 5-MeO-DMT can be administered to BD patients to reduce or eliminate psychomotor retardation in said patients, i.e., to counteract reduced energy and activity and reduced motivation.

In the above mentioned clinical studies involving the administration of 5-MeO-DMT, among others the MADRS item “lassitude” was assessed.

“Lassitude” represents a difficulty getting started or slowness initiating and performing everyday activities.

A score of 0 means that there is hardly any difficulty in getting started and no sluggishness. A score of 2 is assigned if the patient has difficulties in starting activities. A score of 4 means difficulties in starting simple routine activities which are carried out with effort. A score of 6 is assigned in case of complete lassitude, the patient being unable to do anything without help.

This MADRS scale item is of particular relevance to psychomotor retardation, i.e., reduced energy and activity and reduced motivation.

In the study group receiving the individualized dosing regimen, the aggregated score for the MADRS item “lassitude” across all 8 patients was 27 at base line. After 2 hours, it was reduced to 10 which corresponds to an improvement of 17 points or 63%. At day 1 after treatment, it was reduced to 5 which corresponds to an improvement of 22 points or 81%. At day 7 after treatment, it was reduced to 3 which corresponds to an improvement of 24 points or 89%.

In the 12 mg group, the aggregated score for the MADRS item “lassitude” across all 4 patients was 16 at base line. After 2 hours, it was reduced to 10 which corresponds to an improvement of 6 points or 38%. At day 1 after treatment, it was reduced to 0 which corresponds to an improvement of 16 points or 100%. At day 7 after treatment, it was reduced to 3 which corresponds to an improvement of 13 points or 81%.

Thus, the score of the scale item that is of particular relevance to psychomotor retardation, “lassitude”, is markedly improved. The inventors conclude that 5-MeO-DMT can be used to treat psychomotor retardation in patients, in particular patients suffering from mental illness, such as BD, i.e., to counteract reduced energy and activity and reduced motivation.

Consequently, according to the invention, the treatment of a patient suffering from psychomotor retardation reduces or eliminates the psychomotor retardation. The treatment improves or eliminates reduced energy and activity and/or reduced motivation.

The reduction or elimination of psychomotor retardation may be reflected by an improvement in the score of the BDRS items reduced energy and activity and/or reduced motivation about 2 hours; on day 1, for instance, after about 24 hours; on day 7; on day 14; and/or on day 28 after the last administration of 5-MeO-DMT or a pharmaceutically acceptable salt thereof.

The reduction or elimination of psychomotor retardation as reflected by an improvement in the score of the BDRS items reduced energy and activity and/or reduced motivation occurs not later than about 2 hours after the last administration of 5-MeO-DMT or a pharmaceutically acceptable salt thereof. The reduction or elimination of psychomotor retardation as reflected by an improvement in the score of the BDRS items reduced energy and activity and/or reduced motivation preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of psychomotor retardation may be reflected by an improvement in the score of the MADRS item lassitude about 2 hours; on day 1, for instance, after about 24 hours; on day 7; on day 14; and/or on day 28 after the last administration of 5-MeO-DMT or a pharmaceutically acceptable salt thereof.

The reduction or elimination of psychomotor retardation as reflected by an improvement in the score of the MADRS item lassitude occurs not later than about 2 hours after the last administration of 5-MeO-DMT or a pharmaceutically acceptable salt thereof. The reduction or elimination of psychomotor retardation as reflected by an improvement in the score of the MADRS item lassitude preferably persists until at least 6 days; in particular until at least 14 days; more preferably until at least 28 days after the last administration of 5-MeO-DMT or a pharmaceutically acceptable salt thereof.

If the patient suffers from psychomotor retardation the improvement in psychomotor retardation is reflected by a reduction in the Clinical Global Impression-Severity (CGI-S) score after about 2 hours; on day 1, for instance, after about 24 hours; on day 7; on day 14 and/or on day 28 after the last administration of 5-MeO-DMT or a pharmaceutically acceptable salt thereof.

An improvement in psychomotor retardation as reflected by a reduction in the Clinical Global Impression-Severity (CGI-S) score, occurs not later than about 2 hours after the last administration of 5-MeO-DMT or a pharmaceutically acceptable salt thereof.

An improvement in psychomotor retardation, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in psychomotor retardation, as reflected by a reduction in the CGI-S score or by at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, “reduced energy and activity” and “reduced motivation” are items of the BDRS. Since psychomotor retardation furthermore also affects other aspects of BD, the inventors conclude that the observed improvement in the score of the “lassitude” item on the MADRS will yield not only a correlated improvement in the score of the “reduced energy and activity” and/or “reduced motivation” BDRS scale items, but additionally contribute to an overall improvement of the BDRS score.

A further aspect of bipolar disorder, in particular bipolar II disorder, which can be treated by administration of 5-MeO-DMT, is negative thinking, which includes feelings of worthlessness; helplessness and hopelessness; and guilt. 5-MeO-DMT can be administered to BD patients to reduce or eliminate such symptoms in said patients.

Symptoms such as pessimism, feelings of worthlessness and feelings of helplessness and hopelessness have been noted in patients suffering from bipolar disorder. BD patients have also been reported as being more predisposed to feelings of pathological, excessive or inappropriate guilt compared to MDD patients.

These symptoms are clustered together herein as negative thinking.

The MADRS scale item that is of particular relevance to this aspect of BD is “pessimistic thoughts”, which represents thoughts of guilt, inferiority, self-reproach, sinfulness, remorse and ruin.

A score of 0 is assigned if there are no pessimistic thoughts. The score is 2 in case of fluctuating ideas of failure, self-reproach or self-depreciation. A score means persistent self-accusations, or definite but still rational ideas of guilt or sin as well as the patient being increasingly pessimistic about the future. A score of 6 is assigned in case of delusions of ruin, remorse or unredeemable sin and self-accusations which are absurd and unshakable.

In the study group receiving the individualized dosing regimen, the aggregated score for the MADRS item “pessimistic thoughts” across all 8 patients was 28 at base line. After 2 hours, it was reduced to 7 which corresponds to an improvement of 21 points or 75%. At day 1 after treatment, it was reduced to 4 which corresponds to an improvement of 24 points or 86%. At day 7 after treatment, it was reduced to 3 which corresponds to an improvement of 25 points or 89%.

In the 12 mg group, the aggregated score for the MADRS item “pessimistic thoughts” across all 4 patients was 16 at base line. After 2 hours, it was reduced to 8 which corresponds to an improvement of 8 points or 50%. At day 1 after treatment, it was reduced to 7 which corresponds to an improvement of 9 points or 56%. At day 7 after treatment, it was reduced to 8 which corresponds to an improvement of 8 points or 50%.

The BPRS item that is of particular relevance to guilt is “guilt feelings”. This item relates to over concern or remorse for past behaviour. Possible scores are:
1—No guilt feelings.
2—Very Mild. Concerned about having failed someone or at something but not preoccupied. Can shift thoughts to other matters easily.
3—Mild. Concerned about having failed someone or at something with some preoccupation. Tends to voice guilt to others.
4—Moderate. Disproportionate preoccupation with guilt, having done wrong, injured others by doing or failing to do something, but can readily turn attention to other things.
5—Moderately Severe. Preoccupation with guilt, having failed someone or at something, can turn attention to other things, but only with great effort. Not delusional.
6—Severe. Delusional guilt or unreasonable self-reproach very out of proportion to circumstances. Moderate preoccupation present.
7—Extremely Severe. Delusional guilt or unreasonable self-reproach grossly out of proportion to circumstances. Subject is very preoccupied with guilt and is likely to disclose to others or act on delusions.

In the study group receiving the individualized dosing regimen, the aggregated score for the BPRS item “guilt feelings” across all 8 patients was 34 at base line. After 3 hours, it was reduced to 14 which corresponds to an improvement of 20 points or 59%. At day 1 after treatment, it was reduced to 11 which corresponds to an improvement of 23 points or 68%. At day 7 after treatment, it was reduced to 10 which corresponds to an improvement of 24 points or 71%.

In the 12 mg group, the aggregated score for the BPRS item “guilt feelings” across all 4 patients was 18 at base line. After 3 hours, it was reduced to 9 which corresponds to an improvement of 9 points or 50%. At day 1 after treatment, it was reduced to 5 which corresponds to an improvement of 13 points or 72%. At day 7 after treatment, it was reduced to 5 which corresponds to an improvement of 13 points or 72%.

Thus, the score of the MADRS scale item that is of particular relevance to negative thinking, “pessimistic thoughts”, is markedly improved, as is the score of the BPRS item “guilt feelings”. The inventors conclude that 5-MeO-DMT can be used to treat negative thinking in patients, in particular patients suffering from mental illness, such as BD.

Consequently, according to the invention, the treatment of a patient suffering from negative thinking reduces or eliminates the negative thinking. The treatment reduces or eliminates feelings of worthlessness; helplessness and hopelessness; and/or guilt.

The reduction or elimination of negative thinking may be reflected by an improvement at least in the scores of the BDRS items worthlessness; helplessness and hopelessness; and/or guilt about 2 hours; on day 1, for instance, after about 24 hours; on day 7; on day 14; and/or on day 28 after the last administration of 5-MeO-DMT or a pharmaceutically acceptable salt thereof.

The reduction or elimination of negative thinking as reflected by an improvement in the score of the BDRS items worthlessness; helplessness and hopelessness; and/or guilt occurs not later than about 2 hours after the last administration of 5-MeO-DMT or a pharmaceutically acceptable salt thereof. The reduction or elimination of negative thinking as reflected by an improvement in the score of the BDRS items worthlessness; helplessness and hopelessness; and/or guilt preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking may be reflected by an improvement at least in the score of the MADRS item pessimistic thoughts about 2 hours; on day 1, for instance, after about 24 hours; on day 7; on day 14; and/or on day 28 after the last administration of 5-MeO-DMT or a pharmaceutically acceptable salt thereof.

The reduction or elimination of negative thinking as reflected by an improvement in the score of the MADRS item pessimistic thoughts occurs not later than about 2 hours after the last administration of 5-MeO-DMT or a pharmaceutically acceptable salt thereof. The reduction or elimination of negative thinking as reflected by an improvement in the score of the MADRS item pessimistic thoughts preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of negative thinking may be reflected by an improvement at least in the score of the BPRS item guilt feelings about 2 hours; on day 1, for instance, after about 24 hours; on day 7; on day 14; and/or on day 28 after the last administration of 5-MeO-DMT or a pharmaceutically acceptable salt thereof.

The reduction or elimination of negative thinking as reflected by an improvement in the score of the BPRS item guilt feelings occurs not later than about 2 hours after the last administration of 5-MeO-DMT or a pharmaceutically acceptable salt thereof. The reduction or elimination of negative thinking as reflected by an improvement in the score of the BPRS item guilt feelings preferably persists until at least 6 days; in particular until at least 14 days; more preferably until at least 28 days after the last administration of 5-MeO-DMT or a pharmaceutically acceptable salt thereof.

If the patient suffers from negative thinking the improvement in negative thinking is reflected by a reduction in the Clinical Global Impression-Severity (CGI-S) score after about 2 hours; on day 1, for instance, after about 24 hours; on day 7; on day 14 and/or on day 28 after the last administration of 5-MeO-DMT or a pharmaceutically acceptable salt thereof.

An improvement in negative thinking as reflected by a reduction at least in the Clinical Global Impression-Severity (CGI-S) score, occurs not later than about 2 hours after the last administration of 5-MeO-DMT or a pharmaceutically acceptable salt thereof.

An improvement in negative thinking, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in negative thinking, as reflected by a reduction in the CGI-S score or at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, helplessness and hopelessness; worthlessness; and guilt are items of the BDRS. Since negative thinking furthermore also affects other aspects of BD, the inventors conclude that the observed improvement in the score of the “pessimistic thoughts” item on the MADRS and the “Guilt Feelings” item on the BPRS, will yield not only a correlated improvement in the scores of the “worthlessness”, “helplessness and hopelessness” and/or “guilt” BDRS scale items, but additionally contribute to an overall improvement of the BDRS score. For instance, the BDRS item “psychotic symptoms” includes strong feelings of guilt as a contributing factor.

A further aspect of bipolar disorder, in particular bipolar II disorder, which can be treated by administration of 5-MeO-DMT, is anxiety. 5-MeO-DMT can be administered to BD patients to reduce or eliminate feelings of anxiety in said patients

The BPRS item that is of particular relevance to anxiety is “anxiety”. This item relates to reported apprehension, tension, fear, panic or worry. Possible scores are
1—No anxiety
2—Very Mild. Reports some discomfort due to worry or infrequent worries that occur more than usual for most normal individuals.
3—Mild. Worried frequently but can readily turn attention to other things.
4—Moderate. Worried most of the time and cannot turn attention to other things easily but no impairment in functioning or occasional anxiety with autonomic accompaniment but no impairment in functioning.
5—Moderately Severe. Frequent, but not daily, periods of anxiety with autonomic accompaniment or some areas of functioning are disrupted by anxiety or worry.
6—Severe. Anxiety with autonomic accompaniment daily but not persisting throughout the day or many areas of functioning are disrupted by anxiety or constant worry.
7—Extremely Severe. Anxiety with autonomic accompaniment persisting throughout the day or most areas of functioning are disrupted by anxiety or constant worry.

In the study group receiving the individualized dosing regimen, the aggregated score for the BPRS item “anxiety” across all 8 patients was 37 at base line. After 3 hours, it was reduced to 19 which corresponds to an improvement of 18 points or 49%. At day 1 after treatment, it was reduced to 16 which corresponds to an improvement of 21 points or 57%. At day 7 after treatment, it was reduced to 17 which corresponds to an improvement of 20 points or 54%.

In the 12 mg group, the aggregated score for the BPRS item “anxiety” across all 4 patients was 25 at base line. After 3 hours, it was reduced to 11 which corresponds to an improvement of 14 points or 56%. At day 1 after treatment, it was reduced to 6 which corresponds to an improvement of 19 points or 76%. At day 7 after treatment, it was reduced to 6 which corresponds to an improvement of 19 points or 76%.

The inventors conclude that 5-MeO-DMT can be used to treat anxiety in patients, in particular patients suffering from mental illness, such as BD.

Thus, according to the invention, the treatment of a patient suffering from anxiety reduces or eliminates the anxiety.

The reduction or elimination of anxiety may be reflected by an improvement at least in the BDRS item anxiety 2 hours; on day 1, for instance, after about 24 hours; 7 days; 14 days; and/or 28 days after the last administration of 5-MeO-DMT or a pharmaceutically acceptable salt thereof.

The reduction or elimination of anxiety as reflected by an improvement in the BDRS item anxiety occurs not later than about 2 hours after the last administration of 5-MeO-DMT or a pharmaceutically acceptable salt thereof. The reduction or elimination of anxiety as reflected by an improvement in the BDRS item anxiety preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of anxiety is reflected by an improvement at least in the BPRS item anxiety about 2 hours; on day 1, for instance, after about 24 hours; on day 7; on day 14; and/or on day 28 after the last administration of 5-MeO-DMT or a pharmaceutically acceptable salt thereof.

The reduction or elimination of anxiety as reflected by an improvement in the BPRS item anxiety occurs not later than about 2 hours after the last administration of 5-MeO-DMT or a pharmaceutically acceptable salt thereof. The reduction or elimination of anxiety as reflected by an improvement in the BPRS item anxiety preferably persists until at least 6 days; in particular until at least 14 days; more preferably until at least 28 days after the last administration of 5-MeO-DMT or a pharmaceutically acceptable salt thereof.

If the patient suffers from anxiety the improvement in anxiety is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in anxiety as reflected by a reduction in the Clinical Global Impression
Severity (CGI-S) score, occurs not later than about 2 hours after the last administration of 5-MeO-DMT or a pharmaceutically acceptable salt thereof.

An improvement in anxiety, as reflected by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in anxiety, as reflected by as reduction in the CGI-S score or at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, “anxiety” is an item of the BDRS. Since anxiety furthermore also affects other aspects of BD, the inventors conclude that the observed improvement in the “anxiety” item on the BPRS will yield not only a correlated improvement in the “anxiety” BDRS scale item, but additionally contribute to an overall improvement of the BDRS score.

A further aspect of bipolar disorder, in particular bipolar II disorder, which can be treated by administration of 5-MeO-DMT, is cognitive dysfunction, in particular concentration difficulties. 5-MeO-DMT can be administered to BD patients to reduce or eliminate cognitive dysfunction, in particular concentration difficulties, in said patients.

Bipolar depression patients display impairments in the domains of memory and executive functioning, with some evidence of worse executive functioning in bipolar-depressed subjects compared to unipolar-depressed subjects. Additionally, bipolar patients have been reported as having a cognitive component to their psychomotor retardation.

The MADRS item that is of particular relevance to impaired concentration and memory is “concentration difficulties”.

This item represents difficulties in collecting one's thoughts mounting to incapacitating lack of concentration. The score is 0 if the patient has no difficulties in concentrating. The score is 2 in case of occasional difficulties in collecting one's thoughts. A score of 4 is assigned in case of difficulties in concentrating and sustaining thought which reduces ability to read or hold a conversation. The score is 6 if the patient is unable to read or converse without great difficulty.

In the study group receiving the individualized dosing regimen, the aggregated score for the MADRS item “concentration difficulties” across all 8 patients was 30 at base line. After 2 hours, it was reduced to 11 which corresponds to an improvement of 19 points or 63%. At day 1 after treatment, it was reduced to 1 which corresponds to an improvement of 29 points or 97%. At day 7 after treatment, it was reduced to 9 which corresponds to an improvement of 21 points or 70%.

In the 12 mg group, the aggregated score for the MADRS item “concentration difficulties” across all 4 patients was 16 at base line. After 2 hours, it was reduced to 7 which corresponds to an improvement of 9 points or 56%. At day 1 after treatment, it was reduced to 2 which corresponds to an improvement of 14 points or 88%. At day 7 after treatment, it was reduced to 3 which corresponds to an improvement of 13 points or 81%.

Thus, the score of the scale item that is of particular relevance to impaired concentration and memory is markedly improved. The inventors conclude that 5-MeO-DMT can be used to treat impaired concentration and memory in patients, in particular patients suffering from mental illness, such as BD.

Consequently, according to the invention, the treatment of a patient suffering from cognitive dysfunction reduces or eliminates the cognitive dysfunction. The treatment reduces or eliminates impaired concentration and memory.

The reduction or elimination of cognitive dysfunction may be reflected by an improvement at least in the score of the BDRS item impaired concentration and memory about 2 hours; on day 1, for instance, after about 24 hours; on day 7; on day 14; and/or on day 28 after the last administration of 5-MeO-DMT or a pharmaceutically acceptable salt thereof.

The reduction or elimination of cognitive dysfunction as reflected by an improvement in the score of the BDRS item impaired concentration and memory occurs not later than about 2 hours after the last administration of 5-MeO-DMT or a pharmaceutically acceptable salt thereof. The reduction or elimination of cognitive dysfunction as reflected by an improvement in the score of the BDRS item impaired concentration and memory preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of cognitive dysfunction may be reflected by an improvement at least in the score of the MADRS item concentration difficulties about 2 hours; on day 1, for instance, after about 24 hours; on day 7; on day 14; and/or on day 28 after the last administration of 5-MeO-DMT or a pharmaceutically acceptable salt thereof.

The reduction or elimination of cognitive dysfunction as reflected by an improvement in the score of the MADRS item concentration difficulties occurs not later than about 2 hours after the last administration of 5-MeO-DMT or a pharmaceutically acceptable salt thereof. The reduction or elimination of cognitive dysfunction as reflected by an improvement in the score of the MADRS item concentration difficulties preferably persists until at least 6 days; in particular until at least 14 days; more preferably until at least 28 days after the last administration of 5-MeO-DMT or a pharmaceutically acceptable salt thereof.

If the patient suffers from cognitive dysfunction the improvement in cognitive dysfunction is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in cognitive dysfunction as reflected by a reduction in the Clinical Global Impression-Severity (CGI-S) score, occurs not later than about 2 hours after the last administration of 5-MeO-DMT or a pharmaceutically acceptable salt thereof.

An improvement in cognitive dysfunction, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in cognitive dysfunction, as reflected by a reduction of the CGI-S score or at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, “impaired concentration and memory” is an item of the BDRS. Since impaired concentration and memory furthermore also affects other aspects of BD, the inventors conclude that the observed improvement in the score of the “concentration difficulties” item on the MADRS will yield not only a correlated improvement in the score of the “impaired concentration and memory” BDRS scale item, but additionally contribute to an overall improvement of the BDRS score.

A further aspect of bipolar disorder, in particular bipolar II disorder, which can be treated by administration of 5-MeO-DMT, is social/emotional withdrawal or detachment, symptoms of which include anhedonia, emotional withdrawal and loss of affect. 5-MeO-DMT can be administered to BD patients to reduce or eliminate social/emotional withdrawal or detachment in said patients.

Anhedonia (the inability to experience pleasure) has been noted as a key symptom in bipolar disorder.

The scale items that are of particular relevance to social/emotional withdrawal or detachment are “inability to feel”, “emotional withdrawal” and “blunted affect”. The first item is taken from the MADRS, while the latter two are recorded in the BPRS.

The MADRS item “inability to feel” represents the subjective experience of reduced interest in the surroundings, or activities that normally give pleasure. The ability to react with adequate emotion to circumstances or people is reduced.

A score of 0 indicates normal interest in the surroundings and in other people, a score of 2 reduced ability to enjoy usual interests. A score of 4 is assigned in case of a loss of interest in the surroundings and a loss of feelings for friends and acquaintances. A score of 6 reflects the experience of being emotionally paralysed, inability to feel anger, grief or pleasure and a complete or even painful failure to feel for close relatives and friends.

The BPRS item emotional withdrawal relates to a deficiency in the patient's ability to relate emotionally during the interview situation. Possible scores are:
1—No emotional withdrawal.
2—Very Mild. Lack of emotional involvement shown by occasional failure to make reciprocal comments, occasionally appearing preoccupied, or smiling in a stilted manner, but spontaneously engages the interviewer most of the time.
3—Mild. Lack of emotional involvement shown by noticeable failure to make reciprocal comments, appearing preoccupied, or lacking in warmth, but responds to interviewer when approached.
4—Moderate. Emotional contact not present much of the interview because subject does not elaborate responses, fails to make eye contact, doesn't seem to care if interviewer is listening, or may be preoccupied with psychotic material.
5—Moderately Severe. Same as “4” but emotional contact not present most of the interview.
6—Severe. Actively avoids emotional participation. Frequently unresponsive or responds with yes/no answers (not solely due to persecutory delusions). Responds with only minimal affect.
7—Extremely Severe. Consistently avoids emotional participation. Unresponsive or responds with yes/no answers (not solely due to persecutory delusions). May leave during interview or just not respond at all.

The BPRS item blunted affect relates to a restricted range in emotional expressiveness of face, voice, and gestures as well as a marked indifference or flatness even when discussing distressing topics. Possible scores are:
1—No blunted affect.
2—Very Mild. Emotional range is slightly subdued or reserved but displays appropriate facial expressions and tone of voice that are within normal limits.
3—Mild. Emotional range overall is diminished, subdued, or reserved, without many spontaneous and appropriate emotional responses. Voice tone is slightly monotonous.
4—Moderate. Emotional range is noticeably diminished, patient doesn't show emotion, smile, or react to distressing topics except infrequently. Voice tone is monotonous or there is noticeable decrease in spontaneous movements. Displays of emotion or gestures are usually followed by a return to flattened affect.
5—Moderately Severe. Emotional range very diminished, patient doesn't show emotion, smile or react to distressing topics except minimally, few gestures, facial expression does not change very often. Voice tone is monotonous much of the time.
6—Severe. Very little emotional range or expression. Mechanical in speech and gestures most of the time. Unchanging facial expression. Voice tone is monotonous most of the time.
7—Extremely Severe. Virtually no emotional range or expressiveness, stiff movements. Voice tone is monotonous all of the time.

In the study group receiving the individualized dosing regimen, the aggregated score for the MADRS item “inability to feel” across all 8 patients was 36 at base line. After 2 hours, it was reduced to 12 which corresponds to an improvement of 24 points or 67%. At day 1 after treatment, it was reduced to 2 which corresponds to an improvement of 34 points or 94%. At day 7 after treatment, it was reduced to 6 which corresponds to an improvement of 30 points or 83%.

The aggregated score for the BPRS item “emotional withdrawal” was 13 at base line. After 3 hours, it was reduced to 8 which corresponds to an improvement of 5 points or 38%. At day 1 after treatment, it was reduced to 8 which corresponds to an improvement of 5 points or 38%. At day 7 after treatment, it was reduced to 8 which corresponds to an improvement of 5 points or 38%.

The aggregated score for the BPRS item “blunted affect” was 15 at base line. After 3 hours, it was reduced to 11 which corresponds to an improvement of 4 points or 27%. At day 1 after treatment, it was reduced to 8 which corresponds to an improvement of 7 points or 47%. At day 7 after treatment, it was reduced to 8 which corresponds to an improvement of 7 points or 47%.

In the 12 mg group, the aggregated score for the MADRS item “inability to feel” across all 4 patients was 16 at base line. After 2 hours, it was reduced to 9 which corresponds to an improvement of 7 points or 44%. At day 1 after treatment, it was reduced to 1 which corresponds to an improvement of 15 points or 94%. At day 7 after treatment, it was reduced to 1 which corresponds to an improvement of 15 points or 94%.

In the 12 mg group, the aggregated score for the BPRS item “emotional withdrawal” was 13 at base line. After 3 hours, it was reduced to 11 which corresponds to an improvement of 2 points or 15%. At day 1 after treatment, it was reduced to 8 which corresponds to an improvement of 5 points or 38%. At day 7 after treatment, it was reduced to 6 which corresponds to an improvement of 7 points or 54%.

In the 12 mg group, the aggregated score for the BPRS item “blunted affect” was 11 at base line. After 3 hours, it was reduced to 8 which corresponds to an improvement of 3 points or 27%. At day 1 after treatment, it was reduced to 6 which corresponds to an improvement of 5 points or 45%. At day 7 after treatment, it was reduced to 5 which corresponds to an improvement of 6 points or 55%.

Thus, the scores of the scale items that are of particular relevance to social/emotional withdrawal or detachment, namely the MADRS item “inability to feel”, the BPRS item “emotional withdrawal” and the BPRS item “blunted affect”, are markedly improved. The inventors conclude that 5-MeO-DMT can be used to treat social/emotional withdrawal or detachment in patients, in particular patients suffering from mental illness, such as BD, i.e., to counteract “reduced social engagement”, “anhedonia” and/or “affective flattening”.

Consequently, according to the invention, the treatment of a patient suffering from social/emotional withdrawal or detachment reduces or eliminates the social/emotional withdrawal or detachment. The treatment reduces or eliminates at least one of anhedonia, emotional withdrawal and affective flattening.

The reduction or elimination of social/emotional withdrawal or detachment is reflected by an improvement at least in the score of the BDRS items anhedonia, emotional withdrawal and/or affective flattening about 2 hours; on day 1, for instance, after about 24 hours; on day 7; on day 14; and/or on day 28 after the last administration of 5-MeO-DMT or a pharmaceutically acceptable salt thereof.

The reduction or elimination of social/emotional withdrawal or detachment as reflected by an improvement in the score of the BDRS items anhedonia, emotional withdrawal and/or affective flattening occurs not later than about 2 hours after the last administration of 5-MeO-DMT or a pharmaceutically acceptable salt thereof. The reduction or elimination of social/emotional withdrawal or detachment as reflected by an improvement in the score of the BDRS items anhedonia, emotional withdrawal and/or affective flattening preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of social/emotional withdrawal or detachment is reflected by an improvement at least in the score of the MADRS item inability to feel about 2 hours; on day 1, for instance, after about 24 hours; on day 7; on day 14; and/or on day 28 after the last administration of 5-MeO-DMT or a pharmaceutically acceptable salt thereof.

The reduction or elimination of social/emotional withdrawal or detachment as reflected by an improvement in the score of the MADRS item inability to feel occurs not later than about 2 hours after the last administration of 5-MeO-DMT or a pharmaceutically acceptable salt thereof. The reduction or elimination of social/emotional withdrawal or detachment as reflected by an improvement in the score of the MADRS item inability to feel preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of social/emotional withdrawal or detachment is reflected by an improvement at least in the score of the BPRS item emotional withdrawal about 2 hours; on day 1, for instance, after about 24 hours; on day 7; on day 14; and/or on day 28 after the last administration of 5-MeO-DMT or a pharmaceutically acceptable salt thereof.

The reduction or elimination of social/emotional withdrawal or detachment as reflected by an improvement in the score of the BPRS item emotional withdrawal occurs not later than about 2 hours after the last administration of 5-MeO-DMT or a pharmaceutically acceptable salt thereof. The reduction or elimination of social/emotional withdrawal or detachment as reflected by an improvement in the score of the BPRS item emotional withdrawal preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of social/emotional withdrawal or detachment is reflected by an improvement at least in the score of the BPRS item blunted affect about 2 hours; on day 1, for instance, after about 24 hours; on day 7; on day 14; and/or on day 28 after the last administration of 5-MeO-DMT or a pharmaceutically acceptable salt thereof.

The reduction or elimination of social/emotional withdrawal or detachment as reflected by an improvement in the score of the BPRS item blunted affect occurs not later than about 2 hours after the last administration of 5-MeO-DMT or a pharmaceutically acceptable salt thereof. The reduction or elimination of social/emotional withdrawal or detachment as reflected by an improvement in the score of the BPRS item blunted affect preferably persists until at least 6 days; in particular until at least 14 days; more preferably until at least 28 days after the last administration of 5-MeO-DMT or a pharmaceutically acceptable salt thereof.

If the patient suffers from social/emotional withdrawal or detachment the improvement in social/emotional withdrawal or detachment is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in social/emotional withdrawal or detachment as reflected by a reduction in the Clinical Global Impression-Severity (CGI-S) score, occurs not later than about 2 hours after the last administration of 5-MeO-DMT or a pharmaceutically acceptable salt thereof.

An improvement in social/emotional withdrawal or detachment, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in social/emotional withdrawal or detachment, as reflected by a reduction in the CGI-S or at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, “reduced social engagement”, “anhedonia” and “affective flattening” are items of the BDRS. Since social/emotional withdrawal or detachment furthermore also affects other aspects of BD, the inventors conclude that the observed improvement in the score of the “inability to feel” item on the MADRS and in the score of the “emotional withdrawal” and the “blunted affect” on the BPRS will yield not only a correlated improvement in the scores of the “reduced social engagement”, “anhedonia” and/or “affective flattening” BDRS scale items, but additionally contribute to an overall improvement of the BDRS score.

A further aspect of bipolar disorder, in particular bipolar II disorder, which can be treated by administration of 5-MeO-DMT, is suicidal ideation. 5-MeO-DMT can be administered to BD patients to reduce or eliminate suicidal ideation in said patients.

In the above-mentioned clinical studies involving the administration of 5-MeO-DMT, among others the MADRS item “suicidal thoughts” was assessed.

“Suicidal thoughts” represents a feeling that life is not worth living, that a natural death would be welcome, having suicidal thoughts, and/or making the preparations for suicide. Suicidal attempts should not in themselves influence the rating for this MADRS item.

A score of 0 means that the patient enjoys life. A score of 2 is assigned if the patient is weary of life, and/or has only fleeting suicidal thoughts. A score of 4 means the patient feels they would be better off dead, suicidal thoughts are common and suicide is considered as a possible solution but the patient has no specific plans or intention. A score of 6 is assigned in case the patient has explicit plans for suicide and/or is making active preparations.

This MADRS scale item is of particular relevance to suicidal ideation.

In the study group receiving the individualized dosing regimen, the aggregated score for the MADRS item “suicidal thoughts” across all 8 patients was 11 at base line. After 2 hours, it was reduced to 3 which corresponds to an improvement of 8 points or 73%. At day 1 after treatment, it was reduced to 1 which corresponds to an improvement of 10 points or 91%. At day 7 after treatment, it was reduced to 3 which corresponds to an improvement of 8 points or 73%.

In the 12 mg group, the aggregated score for the MADRS item “suicidal thoughts” across all 4 patients was 8 at base line. After 2 hours, it was reduced to 3 which corresponds to an improvement of 5 points or 63%. At day 1 after treatment, it was reduced to 5 which corresponds to an improvement of 3 points or 38%. At day 7 after treatment, it was reduced to 7 which corresponds to an improvement of 1 point or 13%.

Thus, the score of the scale item that is of particular relevance to suicidal ideation, “suicidal thoughts”, is markedly improved, at least in the individualized dosing regimen patients. The inventors conclude that 5-MeO-DMT can be used to treat suicidal ideation in patients, in particular patients suffering from mental illness, such as BD.

Thus, according to the invention, the treatment of a patient suffering from suicidal ideation reduces or eliminates the suicidal ideation.

The reduction or elimination of suicidal ideation may be reflected by an improvement at least in the score of the BDRS item suicidal ideation about 2 hours; on day 1, for instance, after about 24 hours; on day 7; on day 14; and/or on day 28 after the last administration of 5-MeO-DMT or a pharmaceutically acceptable salt thereof.

The reduction or elimination of suicidal ideation as reflected by an improvement in the score of the BDRS item suicidal ideation occurs not later than about 2 hours after the last administration of 5-MeO-DMT or a pharmaceutically acceptable salt thereof. The reduction or elimination of suicidal ideation as reflected by an improvement in the score of the BDRS item suicidal ideation preferably persists until at least 6 days; in particular until at least 14 days; more preferably until at least 28 days after the last administration of 5-MeO-DMT or a pharmaceutically acceptable salt thereof.

Alternatively or additionally, the reduction or elimination of suicidal ideation may be reflected by an improvement at least in the score of the MADRS item suicidal thoughts about 2 hours; on day 1, for instance, after about 24 hours; on day 7; on day 14; and/or on day 28 after the last administration of 5-MeO-DMT or a pharmaceutically acceptable salt thereof.

The reduction or elimination of suicidal ideation as reflected by an improvement in the score of the MADRS item suicidal thoughts occurs not later than about 2 hours after the last administration of 5-MeO-DMT or a pharmaceutically acceptable salt thereof. The reduction or elimination of suicidal ideation as reflected by an improvement in the score of the MADRS item suicidal thoughts preferably persists until at least 6 days; in particular until at least 14 days; more preferably until at least 28 days after the last administration of 5-MeO-DMT or a pharmaceutically acceptable salt thereof.

If the patient suffers from suicidal ideation the improvement in suicidal ideation is reflected by a reduction in the Clinical Global Impression-Severity (CGI-S) score about 2 hours; on day 1, for instance, after about 24 hours; on day 7; on day 14; and/or on day 28 after the last administration of 5-MeO-DMT or a pharmaceutically acceptable salt thereof.

An improvement in suicidal ideation as reflected by a reduction in the Clinical Global Impression-Severity (CGI-S) score, occurs not later than about 2 hours after the last administration of 5-MeO-DMT or a pharmaceutically acceptable salt thereof.

An improvement in suicidal ideation, as assessed by at least a score of “much improved” in the Clinical Global Impression-Improvement (CGI-I) score or the Patient Global Impression-Improvement (PGI-I) score, preferably occurs not later than about 2 hours after the last administration of 5-MeO-DMT or a pharmaceutically acceptable salt thereof.

An improvement in suicidal ideation, as assessed by a reduction of the CGI-S score or at least a score of “much improved” in the CGI-I score or the PGI-I score, preferably persists until at least 6 days; in particular until at least 14 days; more preferably until at least 28 days after the last administration of 5-MeO-DMT or a pharmaceutically acceptable salt thereof.

As indicated above, suicidal ideation is an item of the BDRS. Since Suicidal Ideation furthermore also affects other aspects of BD, the inventors conclude that the observed improvement in the score of the “suicidal thoughts” item on the MADRS will yield not only a correlated improvement in the score of the “suicidal ideation” BDRS scale item, but additionally contribute to an overall improvement of the BDRS score.

A further aspect of bipolar disorder, in particular bipolar II disorder, which can be treated by administration of 5-MeO-DMT, is episodes with mixed features, where the patient may present with the depressive symptoms discussed above but also display symptoms such as psychotic symptoms; irritability; lability; increased motor drive; increased speech, and agitation. The MADRS items of relevance to these additional symptoms include inner tension (58% improvement after 2 hours; 77% improvement at day 1; 54% improvement at day 7 observed in the IDR cohort; 85% improvement after 2 hours; 77% improvement at day 1; 62% improvement at day 7 observed in the 12 mg cohort), which may be linked to irritability and agitation, pessimistic thoughts (reflected by feelings of guilt or delusions of ruin), which may be linked to psychotic symptoms (reflected by pessimism, guilt or delusions) and concentration difficulties, which may be linked to increased speech (reflected by distractibility, among other factors). Furthermore, the BPRS item guilt feelings may be linked to psychotic symptoms, while the BPRS item tension (31% improvement after 3 hours and at day 1 and 38% improvement at day 7 observed in IDR cohort; 36% improvement after 3 hours and 57% improvement at day 1 and at day 7 observed in the 12 mg cohort) may be linked to irritability and agitation.

Improvements in one or more aspects of BD will also lead to overall improvements. Preferably, treatment leads to a remission.

A remission of depressive symptoms may be reflected by a MADRS score equal to or less than 10 and occurs not later than about 2 hours; is observed on day 1, for instance, after about 24 hours; on day 7; on day 14 and/or on day 28 after the last administration of 5-MeO-DMT or a pharmaceutically acceptable salt thereof.

A remission of depressive symptoms may be reflected by a BDRS score equal to or less than 10 and occurs not later than about 2 hours; is observed on day 1, for instance, after about 24 hours; on day 7; on day 14 and/or on day 28 after the last administration of 5-MeO-DMT or a pharmaceutically acceptable salt thereof.

Further alternatively or in addition, a remission of depressive symptoms may be reflected by a HAM-D score equal to or less than 7 and occurs not later than about 2 hours; is observed on day 1, for instance, after about 24 hours; on day 7; on day 14 and/or on day 28 after the last administration of 5-MeO-DMT or a pharmaceutically acceptable salt thereof.

The present invention in particular provides a treatment of depression in BD patients without inducing hypomania or mania. Preferably, the treatment of the present invention reduces or eliminates the risk of the patient developing a hypomanic or manic episode.

The risk of treatment-emergent mania or hypomania (TEM) is an important contributor to clinical trial design and a significant limitation regarding the treatment of bipolar disorder.

The inventors conclude that a treatment with 5-MeO-DMT according to the present invention reduces and/or eliminates the risk of TEM due to several factors related to the compound itself and the manner in which it is administered.

The psychoactive substances reported in relation to TEM are DMT, ayahuasca (containing DMT and MAO inhibitors), psilocybin and LSD. These substances induce a psychoactive active state that builds up over the course of several minutes, lasts for several hours and involves a degree of user engagement with the state itself during which there is ample opportunity for positive and/or negative emotional experiences to occur. This protracted experiential window provides increased opportunity for the occurrence of mania-triggering events.

In contrast, 5-MeO-DMT has a rapid onset of effect (merely seconds) and a duration of typically less than 30 minutes. This provides for a short-lived, albeit intense experience that provides a limited experiential window for the patient.

Furthermore, the nature of the psychoactive phase for 5-MeO-DMT is qualitatively different than that reported for the aforementioned psychoactive substances, in that it results in ego-dissolution or a loss of self, without the sense of cognitive engagement with the experience that accompanies use of the substances previously linked to TEM. The inventors conclude that the deep and intense 5-MeO-DMT experience significantly reduces the risk of triggering mania or hypomania.

Relative to the other psychoactive compounds mentioned previously, 5-MeO-DMT displays an enhanced affinity for the 5-HT1A receptor, where it acts as a potent agonist, whereas the effects of these other compounds are mediated primarily through 5-HT2A agonism. It has been reported that 5-HT1A agonism reduces impulsivity and aggression, whereas 5-HT2A agonism can result in short-term increases in these same traits (Carhart-Harris and Nutt 2017). Furthermore, the dopamine system has been implicated in contributing to mania (Chen 2010), with increased dopamine drive being linked to mania (Berk 2007). LSD, psilocybin and DMT all display increased affinity for a variety of dopamine receptors relative to 5-MeO-DMT (Ray, 2019).

Further, reports of TEM related to psychoactive substance use seem to indicate large quantities of the respective compounds (e.g., DMT/ayahuasca, psilocybin, LSD) were used.

The inventors' approach of sequential up-titration of 5-MeO-DMT significantly reduces the risk of excessive dose administration with its potential for attendant adverse events.

Still further, the induction by antidepressants of isolated events of hypomania has been reported in patients suffering from treatment resistant depression (TRD) (Bader, Cynthia D., and David L. Dunner. “Antidepressant-induced hypomania in treatment-resistant depression.” ·Journal of Psychiatric Practice·13.4 (2007): 233-237). However, the recently concluded clinical trial of 5-MeO-DMT in TRD patients showed no evidence of hypomania induction.

This same clinical trial demonstrated a marked improvement in MADRS scores for reduced sleep, whereas episodes of (hypo) mania are reported to be triggered by reduced sleep, among other factors (Pancheri, 2019).

Thus, when treated according to the invention, the patient does not experience treatment-emergent mania or hypomania.

The occurrence of treatment-emergent mania or hypomania can be assessed using the Young Mania Rating Scale (YMRS). It is considered herein that treatment-emergent mania or hypomania are avoided if the patient has a Young Mania Rating Scale (YMRS) total score less than or equal to 15, preferably less than or equal to 12, as assessed about 2 hours; 1 day; 7 days; 14 days and/or 28 days after the last administration of 5-MeO-DMT or a pharmaceutically acceptable salt thereof.

### Modes of Administration

The therapeutically effective amount of 5-MeO-DMT is administered by intravenous administration, by intramuscular administration or by subcutaneous administration. Administration via these routes can assure a rapid onset of action. A most preferred route of administration is administration via the intravenous route, i.e. by intravenous injection.

5-MeO-DMT can be employed as a pharmaceutically acceptable salt, preferably the hydrobromide salt, or in the form of a formulation for administration via injection, examples of excipients and vehicles for such formulations being known in the art.

### Dosing Regimen

The present invention also provides dose ranges, particular doses as well as dosing regimens (administration schemes) and appropriate routes of administration.

The invention is in part based on the inventors' conclusion that the occurrence of a peak psychedelic experience during the acute phase after administration of 5-MeO-DMT is driving its therapeutic benefit in patients suffering from BD, in particular one or more of the aspects defined above, either in a causal relationship or at least as a surrogate behavioural marker for the underlying unknown therapeutic mechanism.

Consequently, achieving peak experiences more rapidly, in a larger proportion of patients and with better reproducibility in an individual patient, compared with previously tested psychedelic agents, dosing regimens and administration routes, will lead to a better therapeutic profile.

Further, the present invention also relies on the short duration of action of 5-MeO-DMT and the absence of relevant tolerance (i.e., the absence of diminished or no psychedelic effects after re-administration), as a basis for enabling a dosing regimen with frequent re-administrations (such as more than once daily, or daily), which are designed to increase the rate of occurrence of peak experiences, thereby increasing the therapeutic benefit. Such repeat administrations within short time also allow an intraindividual dose-optimization which reduces the risk of overdosing, which may otherwise lead to somatic side effects, such as the serotonin syndrome, negative psychic reactions, such as flashbacks of the experience at later timepoints, induction of mania or hypomania or to less meaningful psychedelic experiences with few or no memories of the altered state (so-called “white-outs”). Further, starting with a low dose allows familiarization of the patient with the psychedelic experience in general, and allows preparation for the more intense symptoms to occur at the higher doses, which will positively influence the experience at those higher doses. Also, the prospect of being able to initiate treatment with a low dose will increase patient acceptance of the therapeutic approach and improve overall compliance rates on the patient population level.

Frequent re-administrations of a serotonergic psychedelic with the aim to increase the rate and tailor the reproducibility of peak experiences and to improve the therapeutic effect, reduce the side effects and improve the compliance rates may not be possible with other psychedelics, due to the late onset and long duration of psychedelic effects and due to the rapid development of tolerance (i.e. diminished or no psychedelic effects after re-administration) which can last for several days.

A patient as defined herein, diagnosed with bipolar disorder, especially bipolar II disorder, and in particular suffering from a current major depressive episode, including a treatment-resistant form of these disorders, and including these disorders associated with suicidal ideation, is treated by administration of 5-MeO-DMT. In a preferred embodiment, the 5-MeO-DMT is administered as a monotherapy, i.e., the patient does not receive any other treatment for BD or symptoms associated with BD.

In preferred embodiments, the dosage amount of 5-MeO-DMT administered to a patient, as defined herein, diagnosed with bipolar disorder, especially bipolar II disorder, and in particular suffering from a current major depressive episode, including a treatment-resistant form of these disorders, and including these disorders associated with suicidal ideation, is in the range of about 1 mg to about 10 mg, or any amount of range therein and is administered in the form of a formulation for administration based on a pharmaceutically acceptable salt of 5-MeO-DMT, such as the hydrobromide salt, which weight amount can be calculated from the stated weight amounts of the 5-MeO-DMT free base, assuming that equimolar amounts are used. Useful specific amounts of 5-MeO-DMT are e.g. about 1 mg, about 2 mg, about 3 mg, about 4 mg, about 5 mg, about 6 mg, about 7 mg, about 8 mg, about 9 mg, and about 10 mg. Note that in this specification, when ranges are set forth, such as “about 1 mg to about 10 mg,” the inventor contemplates all discrete values within that range, some of which are specifically mentioned, but all of which are not-simply for the purpose of brevity.

In preferred embodiments the improved methods for the treatment of a patient, as defined herein, diagnosed with bipolar disorder, especially bipolar II disorder, and in particular suffering from a current major depressive episode, including a treatment-resistant form of these disorders, and including these disorders associated with suicidal ideation, with a therapeutically effective amount of 5-MeO-DMT, comprise the occurrence of a clinical response not later than about 2 hours after administration of 5-MeO-DMT.

In preferred embodiments the improved methods for the treatment of a patient, as defined herein, diagnosed with bipolar disorder, especially bipolar II disorder, and in particular suffering from a current major depressive episode, including a treatment-resistant form of these disorders, and including these disorders associated with suicidal ideation, with a therapeutically effective amount of 5-MeO-DMT, comprise the persistence of a clinical response, including a clinical response which occurred not later than about 2 hours after administration of 5-MeO-DMT, until at least about 6 days after the last administration of 5-MeO-DMT, preferably until at least about 14 days after the last administration of 5-MeO-DMT, more preferably until at least about 28 days after the last administration of 5-MeO-DMT.

In preferred embodiments the improved methods for the treatment of a patient, as defined herein, diagnosed with bipolar disorder, especially bipolar II disorder, and in particular suffering from a current major depressive episode, including a treatment-resistant form of these disorders, and including these disorders associated with suicidal ideation, with a therapeutically effective amount of 5-MeO-DMT comprise the administration of more than a single dose of 5-MeO-DMT.

In a preferred embodiment this more than a single dose of 5-MeO-DMT is administered to a patient in one or more treatment blocks, each block consisting of 2 to 7 administrations, with not less than about 1 hour and not more than about 24 hours between each administration within each treatment block, and not less than about 6 days between the end of one treatment block and the start of the next treatment block.

In an even more preferred embodiment this more than a single dose of 5-MeO-DMT is administered to a patient in one or more treatment blocks, each block consisting of 1 to 3 administrations, with about 24 hours between each administration within each treatment block, and not less than about 6 days between the end of one treatment block and the start of the next treatment block.

In a most preferred embodiment this more than a single dose of 5-MeO-DMT is administered to a patient in one or more treatment blocks, each block consisting of 1 to 3 administrations, with about 1 to 4 hours, preferably 1 to 2 hours, between each administration within each treatment block, and not less than about 6 days between the end of one treatment block and the start of the next treatment block.

In an embodiment the dosage amount of the 5-MeO-DMT administered to an individual patient in each of the administrations and in each of the treatment blocks is constant for that individual patient and is selected from about 1 mg to about 10 mg.

In a preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered.

In an even more preferred embodiment the dosage amount of the 5-MeO-DMT administered to an individual patient is selected from about 1 mg to about 2 mg for the first administration within each treatment block, and then increases with each subsequent administration within each treatment block until the earlier of 10 mg being reached or all administrations within that treatment block being administered or the patient having experienced a peak psychedelic experience or the supervising physician having decided that further dose increases are inappropriate based on observed side effects.

For embodiments where the dosage amount increases for subsequent administrations, the dosage amount for the next administration is determined by adding about 0.25 mg to about 3 mg, preferably about 0.5 mg to about 3 mg to the dosage amount of the prior administration. For example, if the dosage amount of the first administration was 1 mg and the dosage amount increase is 3 mg, unless one of the previously mentioned stopping criteria has been reached, then the dosage amount of the second administration will be 4 mg. Preferably, the dosage amount for the third administration will be 7 mg.

In a preferred embodiment the dosage amount of the 5-MeO-DMT administered to an individual patient in each treatment block is selected from about 1 mg to about 3 mg for the first administration, and then increased, unless the patient has already experienced a peak psychedelic experience within that treatment block or the supervising physician has decided that further dose increases are inappropriate based on observed side effects, to a dosage selected from about 4 mg to about 6 mg for the second administration, and from about 7 mg to about 9 mg for the third administration. Useful specific amounts for the first, second and third administration are e.g. about 2 mg, about 5 mg, and about 8 mg.

In an additional preferred embodiment, the dosage amount of the 5-MeO-DMT administered to an individual patient in each treatment block is selected from about 0.5 mg to about 1.5 mg for the first administration, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration, and from about 2.5 mg to about 3.5 mg for the third administration. Useful specific amounts for the first, second and third administration are e.g. about 1 mg, about 2 mg, and about 3 mg.

In a further preferred embodiment the dosage amount of the 5-MeO-DMT administered to an individual patient is selected from about 1 mg to about 2 mg for the first administration of the first treatment block, and then increases with each subsequent administration within that first treatment block until the earlier of 10 mg being reached or all administrations within that treatment block being administered or the patient having experienced a peak psychedelic experience or the supervising physician having decided that further dose increases are inappropriate based on observed side effects, with that highest dosage in that first treatment block being used as the dosage for all subsequent treatment blocks and administrations within those subsequent treatment blocks. For example, if the highest dosage in the first treatment block was 8 mg because the patient experienced a peak psychedelic experience at that dose, then the dosage for all subsequent treatment blocks and administrations within those subsequent treatment blocks will be 8 mg.

In a particularly preferred embodiment the dosage amount of the 5-MeO-DMT administered to an individual patient is selected from about 1 mg to about 3 mg for the first administration of the first treatment block, and then increased, unless the patient has already experienced a peak psychedelic experience within that treatment block or the supervising physician has decided that further dose increases are inappropriate based on observed side effects, to a dosage selected from about 4 mg to about 6 mg for the second administration of the first treatment block, and from about 7 mg to about 9 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 2 mg, about 5 mg, and about 8 mg.

In a particularly preferred embodiment the dosage amount of the 5-MeO-DMT administered to an individual patient is selected from about 0.5 mg to about 1.5 mg for the first administration of the first treatment block, and then increased, unless the patient has already experienced a peak psychedelic experience within that treatment block or the supervising physician has decided that further dose increases are inappropriate based on observed side effects, to a dosage selected from about 1.5 mg to about 2.5 mg for the second administration of the first treatment block, and from about 2.5 mg to about 3.5 mg for the third administration of the first treatment block, with the highest dosage in that first treatment block being used as the dosage for all subsequent treatment blocks and administrations within those subsequent treatment blocks. Useful specific amounts for the first, second and third administration in the first treatment block are e.g. about 1 mg, about 2 mg, and about 3 mg.

It is understood that a pharmaceutically acceptable salt of 5-MeO-DMT is preferably used in all of the above dosing regimen, and that the appropriate weight amounts of a salt to be administered can be calculated from the stated weight amounts of the free base, assuming that equimolar amounts are used.

According to the invention, 5-MeO-DMT is preferably not administered together with a MAO inhibitor.

The occurrence of a “peak psychedelic experience” in a patient can be identified through achievement of at least 60% of the maximum possible score in each of the four subscales (mystical, positive mood, transcendence of time and space, and ineffability) of the 30-item revised Mystical Experience Questionnaire (MEQ-30) (as described in Barrett F S, J Psychopharmacol. 2015; 29 (11): 1182-90).

The occurrence of a “peak psychedelic experience” in a patient can also be identified through achievement of at least 60% of the maximum possible score of the Oceanic Boundlessness (OBN) dimension of the Altered States of Consciousness (ASC) questionnaire (as described in Roseman L et al., Front Pharmacol. 2018; 8:974).

In accordance with the invention, the occurrence of a “peak psychedelic experience” in a patient is preferably identified through achievement of a score of at least 75 in the Peak Experience Scale (PES) Total Score, also referred to as the Peak Psychedelic Experience Questionnaire (PPEQ), which averages answers scored by the patient from 0 to 100 for the following three questions: 1. How intense was the experience; 2. To what extent did you lose control; 3. How profound (i.e. deep and significant) was the experience?

### ASPECTS OF THE INVENTION

### Aspects

1. 5-Methoxy-N,N-dimethyltryptamine (5-MeO-DMT) or a pharmaceutically acceptable salt thereof for use in treating a patient who is diagnosed with bipolar disorder, wherein the 5-MeO-DMT is administered via the intravenous, intramuscular or subcutaneous route.
2. 5-MeO-DMT or a pharmaceutically acceptable salt thereof for use as in aspect 1, wherein the patient is diagnosed with bipolar II disorder.
3. 5-MeO-DMT or a pharmaceutically acceptable salt thereof for use as in aspect 1, wherein the patient is diagnosed with bipolar I disorder.
4. 5-MeO-DMT or a pharmaceutically acceptable salt thereof for use as in aspects 1 to 3, wherein the patient suffers from a current major depressive episode.
5. 5-MeO-DMT or a pharmaceutically acceptable salt thereof for use as in aspect 4, wherein the patient has a Montgomery-Åsberg Depression Rating Scale (MADRS) total score of equal to or greater than 19, such as equal to or greater than 24, in particular equal to or greater than 37.
6. 5-MeO-DMT or a pharmaceutically acceptable salt thereof for use as in aspect 4 or aspect 5, wherein the patient has a Bipolar Depression Rating Scale (BDRS) total score of equal to or greater than 19, such as equal to or greater than 24, in particular equal to or greater than 37.
7. 5-MeO-DMT or a pharmaceutically acceptable salt thereof for use as in aspects 1 to 6, wherein the patient had no adequate improvement after at least two adequate courses of therapy.
8. 5-MeO-DMT or a pharmaceutically acceptable salt thereof for use as in aspects 1 to 6, wherein the patient had no adequate improvement after at least two adequate courses of therapy, wherein at least one of the two courses was a pharmacotherapy.
9. 5-MeO-DMT or a pharmaceutically acceptable salt thereof for use as in aspects 1 to 6, wherein the patient had no adequate improvement after at least two adequate courses of pharmacotherapy.
10. 5-MeO-DMT or a pharmaceutically acceptable salt thereof for use as in aspects 1 to 9, wherein the patient has a Young Mania Rating Scale (YMRS) total score less than or equal to 8.
11. 5-MeO-DMT or a pharmaceutically acceptable salt thereof for use as in aspects 1 to 10, wherein the 5-MeO-DMT or salt thereof is administered at a dose or in a dosage regimen that causes the patient to experience a peak psychedelic experience.
12. 5-MeO-DMT or a pharmaceutically acceptable salt thereof for use as in aspects 1 to 11, wherein a dosage of about 1 mg to about 10 mg 5-MeO-DMT is administered, or wherein equimolar amounts of the pharmaceutically acceptable salt are administered instead of 5-MeO-DMT.
13. 5-MeO-DMT or a pharmaceutically acceptable salt thereof for use as in aspects 1 to 11, wherein a dosage of about 2 mg; or of about 5 mg; or of about 8 mg is administered, or wherein equimolar amounts of the pharmaceutically acceptable salt are administered instead of 5-MeO-DMT.
14. 5-MeO-DMT or a pharmaceutically acceptable salt thereof for use as in aspects 1 to 11, wherein a dosage of about 1 mg; or of about 2 mg; or of about 3 mg is administered, or wherein equimolar amounts of the pharmaceutically acceptable salt are administered instead of 5-MeO-DMT.
15. 5-MeO-DMT or a pharmaceutically acceptable salt thereof for use as in aspects 1 to 12, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.
16. 5-MeO-DMT or a pharmaceutically acceptable salt thereof for use as in aspects 1 to 12 or 15, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.
17. 5-MeO-DMT or a pharmaceutically acceptable salt thereof for use as in aspect 16, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.
18. 5-MeO-DMT or a pharmaceutically acceptable salt thereof for use as in aspects 1 to 12 or 15, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.
19. 5-MeO-DMT or a pharmaceutically acceptable salt thereof for use as in aspect 18, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.
20. 5-MeO-DMT or a pharmaceutically acceptable salt thereof for use as in aspects 15 to 19, wherein the interval between two administrations is not less than 1 hour and not more than 24 hours, such as about 1 to 4 hours, preferably 1 to 2 hours.
21. 5-MeO-DMT or a pharmaceutically acceptable salt thereof for use as in aspects 11 to 20, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.
22. 5-MeO-DMT or a pharmaceutically acceptable salt thereof for use as in aspect 21, wherein the occurrence of a peak psychedelic experience is identified through achievement of a Peak Experience Scale (PES) Total Score of at least 75.
23. 5-MeO-DMT or a pharmaceutically acceptable salt thereof for use as in any of the prior aspects, wherein the 5-MeO-DMT or a pharmaceutically acceptable salt thereof is administered via intravenous injection.
24. 5-MeO-DMT or a pharmaceutically acceptable salt thereof for use as in aspects 1 to 23, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.
25. 5-MeO-DMT or a pharmaceutically acceptable salt thereof for use as in aspects 1 to 24, wherein a clinical response, as reflected by a reduction in the Clinical Global Impression-Severity (CGI-S) score, is observed on day 1, for instance, about 24 hours after the last administration of 5-MeO-DMT or a pharmaceutically acceptable salt thereof.
26. 5-MeO-DMT or a pharmaceutically acceptable salt thereof for use as in aspects 1 or 25, wherein a clinical response, as reflected by a reduction in the Clinical Global Impression-Severity (CGI-S) score, persists until at least 6 days after the last administration of 5-MeO-DMT or a pharmaceutically acceptable salt thereof.
27. 5-MeO-DMT or a pharmaceutically acceptable salt thereof for use as in aspects 1 to 26, wherein a clinical response, as reflected by a reduction in the Clinical Global Impression-Severity (CGI-S) score, persists until at least 14 days after the last administration of 5-MeO-DMT or a pharmaceutically acceptable salt thereof.
28. 5-MeO-DMT or a pharmaceutically acceptable salt thereof for use as in aspects 1 to 27, wherein a clinical response, as reflected by a reduction in the Clinical Global Impression-Severity (CGI-S) score, persists until at least 28 days after the last administration of 5-MeO-DMT or a pharmaceutically acceptable salt thereof.
29. 5-MeO-DMT or a pharmaceutically acceptable salt thereof for use as in aspects 1 to 28, wherein a clinical response, as reflected by at least 50% improvement of the BDRS; the MADRS or the HAM-D score, compared to the respective score prior to treatment, occurs not later than about 2 hours after the last administration of 5-MeO-DMT or a pharmaceutically acceptable salt thereof.
30. 5-MeO-DMT or a pharmaceutically acceptable salt thereof for use as in aspects 1 to 29, wherein a remission of depressive symptoms, as reflected by a BDRS score equal to or less than 10; a MADRS score equal to or less than 10, or a HAM-D score equal to or less than 7, occurs not later than about 2 hours after the last administration of 5-MeO-DMT or a pharmaceutically acceptable salt thereof.
31. 5-MeO-DMT or a pharmaceutically acceptable salt thereof for use as in aspects 1 to 30, wherein a clinical response, as reflected by at least 50% improvement of the BDRS; the MADRS or the HAM-D score, compared to the respective score prior to treatment, is observed on day 1, for instance, about 24 hours after the last administration of 5-MeO-DMT or a pharmaceutically acceptable salt thereof.
32. 5-MeO-DMT or a pharmaceutically acceptable salt thereof for use as in aspects 1 to 31, wherein a remission of depressive symptoms, as reflected by a BDRS score equal to or less than 10; a MADRS score equal to or less than 10, or a HAM-D score equal to or less than 7, is observed on day 1, for instance, about 24 hours after the last administration of 5-MeO-DMT or a pharmaceutically acceptable salt thereof.
33. 5-MeO-DMT or a pharmaceutically acceptable salt thereof for use as in aspects 1 to 32, wherein a clinical response, as reflected by at least 50% improvement of the BDRS; the MADRS or the HAM-D score, compared to the respective score prior to treatment, persists until at least 6 days after the last administration of 5-MeO-DMT or a pharmaceutically acceptable salt thereof.
34. 5-MeO-DMT or a pharmaceutically acceptable salt thereof for use as in aspects 1 to 33, wherein there is a clinical response, as reflected by at least 75% improvement of the BDRS; the MADRS or the HAM-D score, compared to the respective score prior to treatment, on day 7 after the last administration of 5-MeO-DMT or a pharmaceutically acceptable salt thereof.
35. 5-MeO-DMT or a pharmaceutically acceptable salt thereof for use as in aspects 1 to 34, wherein the patient is in remission of depressive symptoms, as reflected by a BDRS score equal to or less than 10; a MADRS score equal to or less than 10, or a HAM-D score equal to or less than 7, on day 7 after the last administration of 5-MeO-DMT or a pharmaceutically acceptable salt thereof.
36. 5-MeO-DMT or a pharmaceutically acceptable salt thereof for use as in aspects 1 to 35, wherein a clinical response, as reflected by at least 50% improvement of the BDRS; the MADRS or the HAM-D score, compared to the respective score prior to treatment, persists until at least 14 days after the last administration of 5-MeO-DMT or a pharmaceutically acceptable salt thereof.
37. 5-MeO-DMT or a pharmaceutically acceptable salt thereof for use as in aspects 1 to 36, wherein there is a clinical response, as reflected by at least 75% improvement of the BDRS; the MADRS or the HAM-D score, compared to the respective score prior to treatment, on day 14 after the last administration of 5-MeO-DMT or a pharmaceutically acceptable salt thereof.
38. 5-MeO-DMT or a pharmaceutically acceptable salt thereof for use as in aspects 1 to 37, wherein the patient is in remission of depressive symptoms, as reflected by a BDRS score equal to or less than 10; a MADRS score equal to or less than 10, or a HAM-D score equal to or less than 7, on day 14 after the last administration of 5-MeO-DMT or a pharmaceutically acceptable salt thereof.
39. 5-MeO-DMT or a pharmaceutically acceptable salt thereof for use as in aspects 1 to 38, wherein a clinical response, as reflected by at least 50% improvement of the BDRS; the MADRS or the HAM-D score, compared to the respective score prior to treatment, persists until at least 28 days after the last administration of 5-MeO-DMT or a pharmaceutically acceptable salt thereof.
40. 5-MeO-DMT or a pharmaceutically acceptable salt thereof for use as in aspects 1 to 39, wherein there is a clinical response, as reflected by at least 75% improvement of the BDRS; the MADRS or the HAM-D score, compared to the respective score prior to treatment, on day 28 after the last administration of 5-MeO-DMT or a pharmaceutically acceptable salt thereof.
41. 5-MeO-DMT or a pharmaceutically acceptable salt thereof for use as in aspects 1 to 40, wherein the patient is in remission of depressive symptoms, as reflected by a BDRS score equal to or less than 10; a MADRS score equal to or less than 10, or a HAM-D score equal to or less than 7, on day 28 after the last administration of 5-MeO-DMT or a pharmaceutically acceptable salt thereof.
42. 5-MeO-DMT or a pharmaceutically acceptable salt thereof for use as in aspects 1 to 41, wherein the patient does not experience treatment-emergent mania or hypomania.
43. 5-MeO-DMT or a pharmaceutically acceptable salt thereof for use as in aspects 1 to 42, wherein the patient has a Young Mania Rating Scale (YMRS) total score less than or equal to 15, preferably less than or equal to 12, as assessed about 2 hours after the last administration of 5-MeO-DMT or a pharmaceutically acceptable salt thereof.
44. 5-MeO-DMT or a pharmaceutically acceptable salt thereof for use as in aspects 1 to 43, wherein the patient has a Young Mania Rating Scale (YMRS) total score less than or equal to 15, preferably less than or equal to 12, as assessed on day 1, for instance, about 24 hours after the last administration of 5-MeO-DMT or a pharmaceutically acceptable salt thereof.
45. 5-MeO-DMT or a pharmaceutically acceptable salt thereof for use as in aspects 1 to 44, wherein the patient has a Young Mania Rating Scale (YMRS) total score less than or equal to 15, preferably less than or equal to 12, as assessed on day 7 after the last administration of 5-MeO-DMT or a pharmaceutically acceptable salt thereof.
46. 5-MeO-DMT or a pharmaceutically acceptable salt thereof for use as in aspects 1 to 45, wherein the patient has a Young Mania Rating Scale (YMRS) total score less than or equal to 15, preferably less than or equal to 12, as assessed on day 14 after the last administration of 5-MeO-DMT or a pharmaceutically acceptable salt thereof.
47. 5-MeO-DMT or a pharmaceutically acceptable salt thereof for use as in aspects 1 to 46, wherein the patient has a Young Mania Rating Scale (YMRS) total score less than or equal to 15, preferably less than or equal to 12, as assessed on day 28 after the last administration of 5-MeO-DMT or a pharmaceutically acceptable salt thereof.
48. 5-MeO-DMT or a pharmaceutically acceptable salt thereof for use as in aspects 1 to 47, wherein the treatment leads to an improvement in at least one of sleep disturbance, psychomotor retardation, negative thinking, anxiety, cognitive dysfunction, and social/emotional withdrawal or detachment.
49. 5-MeO-DMT or a pharmaceutically acceptable salt thereof for use as in aspects 1 to 48, wherein the patient suffers from sleep disturbance and the treatment reduces or eliminates the sleep disturbance.
50. 5-MeO-DMT or a pharmaceutically acceptable salt thereof for use as in aspect 49, wherein the patient suffers from insomnia and the treatment reduces or eliminates insomnia.
51. 5-MeO-DMT or a pharmaceutically acceptable salt thereof for use as in aspect 49, wherein the patient suffers from hypersomnia and the treatment reduces or eliminates hypersomnia.
52. 5-MeO-DMT or a pharmaceutically acceptable salt thereof for use as in aspects 49 to 51, wherein the reduction or elimination of sleep disturbance is reflected by an improvement at least in the score of the BDRS item sleep disturbance on day 1, for instance, about 24 hours after the last administration of 5-MeO-DMT or a pharmaceutically acceptable salt thereof.
53. 5-MeO-DMT or a pharmaceutically acceptable salt thereof for use as in aspects 49 to 52, wherein the reduction or elimination of sleep disturbance is reflected by an improvement at least in the score of the BDRS item sleep disturbance on day 7 after the last administration of 5-MeO-DMT or a pharmaceutically acceptable salt thereof.
54. 5-MeO-DMT or a pharmaceutically acceptable salt thereof for use as in aspects 49 to 53, wherein the reduction or elimination of sleep disturbance is reflected by an improvement at least in the score of the BDRS item sleep disturbance on day 14 after the last administration of 5-MeO-DMT or a pharmaceutically acceptable salt thereof.
55. 5-MeO-DMT or a pharmaceutically acceptable salt thereof for use as in aspects 49 to 54, wherein the reduction or elimination of sleep disturbance is reflected by an improvement at least in the score of the BDRS item sleep disturbance on day 28 after the last administration of 5-MeO-DMT or a pharmaceutically acceptable salt thereof.
56. 5-MeO-DMT or a pharmaceutically acceptable salt thereof for use as in aspect 49, wherein the reduction or elimination of sleep disturbance as reflected by an improvement in the score of the BDRS item sleep disturbance occurs not later than about 24 hours after the last administration of 5-MeO-DMT or a pharmaceutically acceptable salt thereof.
57. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 6 days after the last administration of 5-MeO-DMT or a pharmaceutically acceptable salt thereof.
58. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 14 days after the last administration of 5-MeO-DMT or a pharmaceutically acceptable salt thereof.
59. 5-MeO-DMT or a pharmaceutically acceptable salt thereof for use as in aspect 56, wherein the reduction or elimination of sleep disturbance as reflected by an improvement in the score of the BDRS item sleep disturbance persists until at least 28 days after the last administration of 5-MeO-DMT or a pharmaceutically acceptable salt thereof.
60. 5-MeO-DMT or a pharmaceutically acceptable salt thereof for use as in aspect 49, wherein the sleep disturbance is reduced sleep and the reduction or elimination of sleep disturbance is reflected by an improvement at least in the score of the MADRS item reduced sleep on day 1, for instance, about 24 hours after the last administration of 5-MeO-DMT or a pharmaceutically acceptable salt thereof.
61. 5-MeO-DMT or a pharmaceutically acceptable salt thereof for use as in aspects 49 or 60, wherein the sleep disturbance is reduced sleep and the reduction or elimination of sleep disturbance is reflected by an improvement at least in the score of the MADRS item reduced sleep on day 7 after the last administration of 5-MeO-DMT or a pharmaceutically acceptable salt thereof.
62. 5-MeO-DMT or a pharmaceutically acceptable salt thereof for use as in aspects 49, 60 or 61, wherein the sleep disturbance is reduced sleep and the reduction or elimination of sleep disturbance is reflected by an improvement at least in the score of the MADRS item reduced sleep on day 14 after the last administration of 5-MeO-DMT or a pharmaceutically acceptable salt thereof.
63. 5-MeO-DMT or a pharmaceutically acceptable salt thereof for use as in aspects 49 or 60 to 62, wherein the sleep disturbance is reduced sleep and the reduction or elimination of sleep disturbance is reflected by an improvement at least in the score of the MADRS item reduced sleep on day 28 after the last administration of 5-MeO-DMT or a pharmaceutically acceptable salt thereof.
64. 5-MeO-DMT or a pharmaceutically acceptable salt thereof for use as in aspect 49, wherein the sleep disturbance is reduced sleep and the reduction or elimination of sleep disturbance as reflected by an improvement in the score of the MADRS item reduced sleep occurs not later than about 24 hours after the last administration of 5-MeO-DMT or a pharmaceutically acceptable salt thereof.
65. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 6 days after the last administration of 5-MeO-DMT or a pharmaceutically acceptable salt thereof.
66. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 14 days after the last administration of 5-MeO-DMT or a pharmaceutically acceptable salt thereof.
67. 5-MeO-DMT or a pharmaceutically acceptable salt thereof for use as in aspect 64, wherein the sleep disturbance is reduced sleep and the reduction or elimination of sleep disturbance as reflected by an improvement in the score of the MADRS item reduced sleep persists until at least 28 days after the last administration of 5-MeO-DMT or a pharmaceutically acceptable salt thereof.
68. 5-MeO-DMT or a pharmaceutically acceptable salt thereof for use as in aspect 49, wherein the patient suffers from sleep disturbance and an improvement in sleep disturbance, is reflected by a reduction in the Clinical Global Impression-Severity (CGI-S) score on day 1, for instance, about 24 hours after the last administration of 5-MeO-DMT or a pharmaceutically acceptable salt thereof.
69. 5-MeO-DMT or a pharmaceutically acceptable salt thereof for use as in aspects 49 or 68, wherein the patient suffers from sleep disturbance and an improvement in sleep disturbance is reflected by a reduction in the Clinical Global Impression-Severity (CGI-S) score on day 7 after the last administration of 5-MeO-DMT or a pharmaceutically acceptable salt thereof.
70. 5-MeO-DMT or a pharmaceutically acceptable salt thereof for use as in aspects 49, 68 or 69, wherein the patient suffers from sleep disturbance and an improvement in sleep disturbance is reflected by a reduction in the Clinical Global Impression-Severity (CGI-S) score on day 14 after the last administration of 5-MeO-DMT or a pharmaceutically acceptable salt thereof.
71. 5-MeO-DMT or a pharmaceutically acceptable salt thereof for use as in aspects 49 or 68 to 70, wherein the patient suffers from sleep disturbance and an improvement in sleep disturbance is reflected by a reduction in the Clinical Global Impression-Severity (CGI-S) score on day 28 after the last administration of 5-MeO-DMT or a pharmaceutically acceptable salt thereof.
72. 5-MeO-DMT or a pharmaceutically acceptable salt thereof for use as in aspects 1 to 48, wherein the patient suffers from sleep disturbance and an improvement in sleep disturbance, as reflected by a reduction in the Clinical Global Impression-Severity (CGI-S) score, occurs not later than about 24 hours after the last administration of 5-MeO-DMT or a pharmaceutically acceptable salt thereof.
73. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 6 days after the last administration of 5-MeO-DMT or a pharmaceutically acceptable salt thereof.
74. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 14 days after the last administration of 5-MeO-DMT or a pharmaceutically acceptable salt thereof.
75. 5-MeO-DMT or a pharmaceutically acceptable salt thereof for use as in aspect 72, wherein the improvement in sleep disturbance, as reflected by a reduction in the Clinical Global Impression-Severity (CGI-S) score, persists until at least 28 days after the last administration of 5-MeO-DMT or a pharmaceutically acceptable salt thereof.
76. 5-MeO-DMT or a pharmaceutically acceptable salt thereof for use as in aspects 1 to 48, wherein the patient suffers from sleep disturbance and the reduction or elimination of sleep disturbance is reflected by an improvement in the Pittsburgh Sleep Quality Index (PSQI) global score on day 1, for instance, about 24 hours after the last administration of 5-MeO-DMT or a pharmaceutically acceptable salt thereof, wherein the recall period spans from the time point when acute psychedelic experiences have subsided after the last administration to the assessment time point.
77. 5-MeO-DMT or a pharmaceutically acceptable salt thereof for use as in aspects 1 to 48 or 76, wherein the patient suffers from sleep disturbance and the reduction or elimination of sleep disturbance is reflected by an improvement in the Pittsburgh Sleep Quality Index (PSQI) global score on day 7 after the last administration of 5-MeO-DMT or a pharmaceutically acceptable salt thereof, wherein the recall period spans from the time point when acute psychedelic experiences have subsided after the last administration to the assessment time point.
78. 5-MeO-DMT or a pharmaceutically acceptable salt thereof for use as in aspects 1 to 48, 76 or 77, wherein the patient suffers from sleep disturbance and the reduction or elimination of sleep disturbance is reflected by an improvement in the Pittsburgh Sleep Quality Index (PSQI) global score on day 14 after the last administration of 5-MeO-DMT or a pharmaceutically acceptable salt thereof, wherein the recall period spans from the time point when acute psychedelic experiences have subsided after the last administration to the assessment time point.
79. 5-MeO-DMT or a pharmaceutically acceptable salt thereof for use as in aspects 1 to 48 or 76 to 78, wherein the patient suffers from sleep disturbance and the reduction or elimination of sleep disturbance is reflected by an improvement in the Pittsburgh Sleep Quality Index (PSQI) global score on day 28 after the last administration of 5-MeO-DMT or a pharmaceutically acceptable salt thereof, wherein the recall period spans from the time point when acute psychedelic experiences have subsided after the last administration to the assessment time point.
80. 5-MeO-DMT or a pharmaceutically acceptable salt thereof for use as in aspect 49, wherein the patient suffers from sleep disturbance and the reduction or elimination of sleep disturbance as reflected by an improvement in the Pittsburgh Sleep Quality Index (PSQI) global score occurs not later than about 24 hours after the last administration of 5-MeO-DMT or a pharmaceutically acceptable salt thereof, wherein the recall period spans from the time point when acute psychedelic experiences have subsided after the last administration to the assessment time point.
81. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 6 days after the last administration of 5-MeO-DMT or a pharmaceutically acceptable salt thereof, wherein the recall period spans from the time point when acute psychedelic experiences have subsided after the last administration to the assessment time point.
82. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 14 days after the last administration of 5-MeO-DMT or a pharmaceutically acceptable salt thereof, wherein the recall period spans from the time point when acute psychedelic experiences have subsided after the last administration to the assessment time point.
83. 5-MeO-DMT or a pharmaceutically acceptable salt thereof for use as in aspect 80, wherein the patient suffers from sleep disturbance and the reduction or elimination of sleep disturbance as reflected by an improvement in the Pittsburgh Sleep Quality Index (PSQI) global score persists until at least 28 days after the last administration of 5-MeO-DMT or a pharmaceutically acceptable salt thereof, wherein the recall period spans from the time point when acute psychedelic experiences have subsided after the last administration to the assessment time point.
84. 5-MeO-DMT or a pharmaceutically acceptable salt thereof for use as in aspects 1 to 83, wherein the patient suffers from psychomotor retardation and the treatment reduces or eliminates the psychomotor retardation.
85. 5-MeO-DMT or a pharmaceutically acceptable salt thereof for use as in aspect 84, wherein the treatment improves or eliminates reduced energy and activity and/or reduced motivation.
86. 5-MeO-DMT or a pharmaceutically acceptable salt thereof for use as in aspects 84 or 85, wherein the reduction or elimination of psychomotor retardation is reflected by an improvement at least in the score of the BDRS items reduced energy and activity and/or reduced motivation about 2 hours after the last administration of 5-MeO-DMT or a pharmaceutically acceptable salt thereof.
87. 5-MeO-DMT or a pharmaceutically acceptable salt thereof for use as in aspects 84 to 86, wherein the reduction or elimination of psychomotor retardation is reflected by an improvement at least in the score of the BDRS items reduced energy and activity and/or reduced motivation on day 1, for instance, about 24 hours after the last administration of 5-MeO-DMT or a pharmaceutically acceptable salt thereof.
88. 5-MeO-DMT or a pharmaceutically acceptable salt thereof for use as in aspects 84 to 87, wherein the reduction or elimination of psychomotor retardation is reflected by an improvement at least in the score of the BDRS items reduced energy and activity and/or reduced motivation on day 7 after the last administration of 5-MeO-DMT or a pharmaceutically acceptable salt thereof.
89. 5-MeO-DMT or a pharmaceutically acceptable salt thereof for use as in aspects 84 to 88, wherein the reduction or elimination of psychomotor retardation is reflected by an improvement at least in the score of the BDRS items reduced energy and activity and/or reduced motivation on day 14 after the last administration of 5-MeO-DMT or a pharmaceutically acceptable salt thereof.
90. 5-MeO-DMT or a pharmaceutically acceptable salt thereof for use as in aspects 84 to 89, wherein the reduction or elimination of psychomotor retardation is reflected by an improvement at least in the score of the BDRS items reduced energy and activity and/or reduced motivation on day 28 after the last administration of 5-MeO-DMT or a pharmaceutically acceptable salt thereof.
91. 5-MeO-DMT or a pharmaceutically acceptable salt thereof for use as in aspects 84 or 85, wherein the reduction or elimination of psychomotor retardation as reflected by an improvement in the score of the BDRS items reduced energy and activity and/or reduced motivation occurs not later than about 2 hours after the last administration of 5-MeO-DMT or a pharmaceutically acceptable salt thereof.
92. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 6 days after the last administration of 5-MeO-DMT or a pharmaceutically acceptable salt thereof.
93. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 14 days after the last administration of 5-MeO-DMT or a pharmaceutically acceptable salt thereof.
94. 5-MeO-DMT or a pharmaceutically acceptable salt thereof for use as in aspect 91, wherein the reduction or elimination of psychomotor retardation as reflected by an improvement in the score of the BDRS items reduced energy and activity and/or reduced motivation persists until at least 28 days after the last administration of 5-MeO-DMT or a pharmaceutically acceptable salt thereof.
95. 5-MeO-DMT or a pharmaceutically acceptable salt thereof for use as in aspect 84, wherein the reduction or elimination of psychomotor retardation is reflected by an improvement at least in the score of the MADRS item lassitude about 2 hours after the last administration of 5-MeO-DMT or a pharmaceutically acceptable salt thereof.
96. 5-MeO-DMT or a pharmaceutically acceptable salt thereof for use as in aspects 84 or 95, wherein the reduction or elimination of psychomotor retardation is reflected by an improvement at least in the score of the MADRS item lassitude on day 1, for instance, about 24 hours after the last administration of 5-MeO-DMT or a pharmaceutically acceptable salt thereof.
97. 5-MeO-DMT or a pharmaceutically acceptable salt thereof for use as in aspects 84, 95 or 96, wherein the reduction or elimination of psychomotor retardation is reflected by an improvement at least in the score of the MADRS item lassitude on day 7 after the last administration of 5-MeO-DMT or a pharmaceutically acceptable salt thereof.
98. 5-MeO-DMT or a pharmaceutically acceptable salt thereof for use as in aspects 84 or 95 to 97, wherein the reduction or elimination of psychomotor retardation is reflected by an improvement at least in the score of the MADRS item lassitude on day 14 after the last administration of 5-MeO-DMT or a pharmaceutically acceptable salt thereof.
99. 5-MeO-DMT or a pharmaceutically acceptable salt thereof for use as in aspects 84 or 95 to 98, wherein the reduction or elimination of psychomotor retardation is reflected by an improvement at least in the score of the MADRS item lassitude on day 28 after the last administration of 5-MeO-DMT or a pharmaceutically acceptable salt thereof.
100. 5-MeO-DMT or a pharmaceutically acceptable salt thereof for use as in aspect 84, wherein the reduction or elimination of psychomotor retardation as reflected by an improvement in the score of the MADRS item lassitude occurs not later than about 2 hours after the last administration of 5-MeO-DMT or a pharmaceutically acceptable salt thereof.
101. 5-MeO-DMT or a pharmaceutically acceptable salt thereof for use as in aspect 100, wherein the reduction or elimination of psychomotor retardation as reflected by an improvement in the score of the MADRS item lassitude persists until at least 6 days after the last administration of 5-MeO-DMT or a pharmaceutically acceptable salt thereof.
102. 5-MeO-DMT or a pharmaceutically acceptable salt thereof for use as in aspect 100, wherein the reduction or elimination of psychomotor retardation as reflected by an improvement in the score of the MADRS item lassitude persists until at least 14 days after the last administration of 5-MeO-DMT or a pharmaceutically acceptable salt thereof.
103. 5-MeO-DMT or a pharmaceutically acceptable salt thereof for use as in aspect 100, wherein the reduction or elimination of psychomotor retardation as reflected by an improvement in the score of the MADRS item lassitude persists until at least 28 days after the last administration of 5-MeO-DMT or a pharmaceutically acceptable salt thereof.
104. 5-MeO-DMT or a pharmaceutically acceptable salt thereof for use as in aspect 84, wherein the patient suffers from psychomotor retardation and an improvement in psychomotor retardation is reflected by a reduction in the Clinical Global Impression-Severity (CGI-S) score about 2 hours after the last administration of 5-MeO-DMT or a pharmaceutically acceptable salt thereof.
105. 5-MeO-DMT or a pharmaceutically acceptable salt thereof for use as in aspects 84 or 104, wherein the patient suffers from psychomotor retardation and an improvement in psychomotor retardation is reflected by a reduction in the Clinical Global Impression-Severity (CGI-S) score on day 1, for instance, about 24 hours after the last administration of 5-MeO-DMT or a pharmaceutically acceptable salt thereof.
106. 5-MeO-DMT or a pharmaceutically acceptable salt thereof for use as in aspects 84, 104 or 105, wherein the patient suffers from psychomotor retardation and an improvement in psychomotor retardation is reflected by a reduction in the Clinical Global Impression-Severity (CGI-S) score on day 7 after the last administration of 5-MeO-DMT or a pharmaceutically acceptable salt thereof.
107. 5-MeO-DMT or a pharmaceutically acceptable salt thereof for use as in aspects 84 or 104 to 106, wherein the patient suffers from psychomotor retardation and an improvement in psychomotor retardation is reflected by a reduction in the Clinical Global Impression-Severity (CGI-S) score on day 14 after the last administration of 5-MeO-DMT or a pharmaceutically acceptable salt thereof.
108. 5-MeO-DMT or a pharmaceutically acceptable salt thereof for use as in aspects 84 or 104 to 107, wherein the patient suffers from psychomotor retardation and an improvement in psychomotor retardation is reflected by a reduction in the Clinical Global Impression-Severity (CGI-S) score on day 28 after the last administration of 5-MeO-DMT or a pharmaceutically acceptable salt thereof.
109. 5-MeO-DMT or a pharmaceutically acceptable salt thereof for use as in aspects 1 to 83, wherein the patient suffers from psychomotor retardation and an improvement in psychomotor retardation, as reflected by a reduction in the Clinical Global Impression-Severity (CGI-S) score, occurs not later than about 2 hours after the last administration of 5-MeO-DMT or a pharmaceutically acceptable salt thereof.
110. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 6 days after the last administration of 5-MeO-DMT or a pharmaceutically acceptable salt thereof.
111. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 14 days after the last administration of 5-MeO-DMT or a pharmaceutically acceptable salt thereof.
112. 5-MeO-DMT or a pharmaceutically acceptable salt thereof for use as in aspect 109, wherein the improvement in psychomotor retardation, as reflected by a reduction in the Clinical Global Impression-Severity (CGI-S) score, persists until at least 28 days after the last administration of 5-MeO-DMT or a pharmaceutically acceptable salt thereof.
113. 5-MeO-DMT or a pharmaceutically acceptable salt thereof for use as in aspects 1 to 112, wherein the patient suffers from negative thinking and the treatment reduces or eliminates the negative thinking.
114. 5-MeO-DMT or a pharmaceutically acceptable salt thereof for use as in aspect 113, wherein the treatment reduces or eliminates feelings of worthlessness; helplessness and hopelessness; and/or guilt.
115. 5-MeO-DMT or a pharmaceutically acceptable salt thereof for use as in aspects 113 or 114, wherein the reduction or elimination of negative thinking is reflected by an improvement at least in the scores of the BDRS items worthlessness; helplessness and hopelessness; and/or guilt about 2 hours after the last administration of 5-MeO-DMT or a pharmaceutically acceptable salt thereof.
116. 5-MeO-DMT or a pharmaceutically acceptable salt thereof for use as in aspects 113 to 115, wherein the reduction or elimination of negative thinking is reflected by an improvement at least in the scores of the BDRS items worthlessness; helplessness and hopelessness; and/or guilt on day 1, for instance, about 24 hours after the last administration of 5-MeO-DMT or a pharmaceutically acceptable salt thereof.
117. 5-MeO-DMT or a pharmaceutically acceptable salt thereof for use as in aspects 113 to 116, wherein the reduction or elimination of negative thinking is reflected by an improvement at least in the scores of the BDRS items worthlessness; helplessness and hopelessness; and/or guilt on day 7 after the last administration of 5-MeO-DMT or a pharmaceutically acceptable salt thereof.
118. 5-MeO-DMT or a pharmaceutically acceptable salt thereof for use as in aspects 113 to 117, wherein the reduction or elimination of negative thinking is reflected by an improvement at least in the scores of the BDRS items worthlessness; helplessness and hopelessness; and/or guilt on day 14 after the last administration of 5-MeO-DMT or a pharmaceutically acceptable salt thereof.
119. 5-MeO-DMT or a pharmaceutically acceptable salt thereof for use as in aspects 113 to 118, wherein the reduction or elimination of negative thinking is reflected by an improvement at least in the score of the BDRS items worthlessness; helplessness and hopelessness; and/or guilt on day 28 after the last administration of 5-MeO-DMT or a pharmaceutically acceptable salt thereof.
120. 5-MeO-DMT or a pharmaceutically acceptable salt thereof for use as in aspects 113 or 114, wherein the reduction or elimination of negative thinking as reflected by an improvement in the scores of the BDRS items worthlessness; helplessness and hopelessness; and/or guilt occurs not later than about 2 hours after the last administration of 5-MeO-DMT or a pharmaceutically acceptable salt thereof.
121. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 6 days after the last administration of 5-MeO-DMT or a pharmaceutically acceptable salt thereof.
122. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 14 days after the last administration of 5-MeO-DMT or a pharmaceutically acceptable salt thereof.
123. 5-MeO-DMT or a pharmaceutically acceptable salt thereof for use as in aspect 120, wherein the reduction or elimination of negative thinking as reflected by an improvement in the scores of the BDRS items worthlessness; helplessness and hopelessness; and/or guilt persists until at least 28 days after the last administration of 5-MeO-DMT or a pharmaceutically acceptable salt thereof.
124. 5-MeO-DMT or a pharmaceutically acceptable salt thereof for use as in aspects 113 or 114, wherein the reduction or elimination of negative thinking is reflected by an improvement at least in the score of the MADRS item pessimistic thoughts about 2 hours after the last administration of 5-MeO-DMT or a pharmaceutically acceptable salt thereof.
125. 5-MeO-DMT or a pharmaceutically acceptable salt thereof for use as in aspects 113, 114 or 124, wherein the reduction or elimination of negative thinking is reflected by an improvement at least in the score of the MADRS item pessimistic thoughts on day 1, for instance, about 24 hours after the last administration of 5-MeO-DMT or a pharmaceutically acceptable salt thereof.
126. 5-MeO-DMT or a pharmaceutically acceptable salt thereof for use as in aspects 113, 114, 124 or 125, wherein the reduction or elimination of negative thinking is reflected by an improvement at least in the score of the MADRS item pessimistic thoughts on day 7 after the last administration of 5-MeO-DMT or a pharmaceutically acceptable salt thereof.
127. 5-MeO-DMT or a pharmaceutically acceptable salt thereof for use as in aspects 113, 114 or 124 to 126, wherein the reduction or elimination of negative thinking is reflected by an improvement at least in the score of the MADRS item pessimistic thoughts on day 14 after the last administration of 5-MeO-DMT or a pharmaceutically acceptable salt thereof.
128. 5-MeO-DMT or a pharmaceutically acceptable salt thereof for use as in aspects 113, 114 or 124 to 127, wherein the reduction or elimination of negative thinking is reflected by an improvement at least in the score of the MADRS item pessimistic thoughts on day 28 after the last administration of 5-MeO-DMT or a pharmaceutically acceptable salt thereof.
129. 5-MeO-DMT or a pharmaceutically acceptable salt thereof for use as in aspects 113 or 114, wherein the reduction or elimination of negative thinking as reflected by an improvement in the score of the MADRS item pessimistic thoughts occurs not later than about 2 hours after the last administration of 5-MeO-DMT or a pharmaceutically acceptable salt thereof.
130. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 6 days after the last administration of 5-MeO-DMT or a pharmaceutically acceptable salt thereof.
131. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 14 days after the last administration of 5-MeO-DMT or a pharmaceutically acceptable salt thereof.
132. 5-MeO-DMT or a pharmaceutically acceptable salt thereof for use as in aspect 129, wherein the reduction or elimination of negative thinking as reflected by an improvement in the score of the MADRS item pessimistic thoughts persists until at least 28 days after the last administration of 5-MeO-DMT or a pharmaceutically acceptable salt thereof.
133. 5-MeO-DMT or a pharmaceutically acceptable salt thereof for use as in aspect 113 or 114, wherein the reduction or elimination of negative thinking is reflected by an improvement at least in the score of the BPRS item guilt feelings about 2 hours after the last administration of 5-MeO-DMT or a pharmaceutically acceptable salt thereof.
134. 5-MeO-DMT or a pharmaceutically acceptable salt thereof for use as in aspects 113, 114 or 133, wherein the reduction or elimination of negative thinking is reflected by an improvement at least in the score of the BPRS item guilt feelings on day 1, for instance, about 24 hours after the last administration of 5-MeO-DMT or a pharmaceutically acceptable salt thereof.
135. 5-MeO-DMT or a pharmaceutically acceptable salt thereof for use as in aspects 113, 114, 133 or 134, wherein the reduction or elimination of negative thinking is reflected by an improvement at least in the score of the BPRS item guilt feelings on day 7 after the last administration of 5-MeO-DMT or a pharmaceutically acceptable salt thereof.
136. 5-MeO-DMT or a pharmaceutically acceptable salt thereof for use as in aspects 113, 114 or 133 to 135, wherein the reduction or elimination of negative thinking is reflected by an improvement at least in the score of the BPRS item guilt feelings on day 14 after the last administration of 5-MeO-DMT or a pharmaceutically acceptable salt thereof.
137. 5-MeO-DMT or a pharmaceutically acceptable salt thereof for use as in aspects 113, 114 or 133 to 136 wherein the reduction or elimination of negative thinking is reflected by an improvement at least in the score of the BPRS item guilt feelings on day 28 after the last administration of 5-MeO-DMT or a pharmaceutically acceptable salt thereof.
138. 5-MeO-DMT or a pharmaceutically acceptable salt thereof for use as in aspects 113 or 114, wherein the reduction or elimination of negative thinking as reflected by an improvement in the score of the BPRS item guilt feelings occurs not later than about 2 hours after the last administration of 5-MeO-DMT or a pharmaceutically acceptable salt thereof.
139. 5-MeO-DMT or a pharmaceutically acceptable salt thereof for use as in aspect 138, wherein the reduction or elimination of negative thinking as reflected by an improvement in the score of the BPRS item guilt feelings persists until at least 6 days after the last administration of 5-MeO-DMT or a pharmaceutically acceptable salt thereof.
140. 5-MeO-DMT or a pharmaceutically acceptable salt thereof for use as in aspect 138, wherein the reduction or elimination of negative thinking as reflected by an improvement in the score of the BPRS item guilt feelings persists until at least 14 days after the last administration of 5-MeO-DMT or a pharmaceutically acceptable salt thereof.
141. 5-MeO-DMT or a pharmaceutically acceptable salt thereof for use as in aspect 138, wherein the reduction or elimination of negative thinking as reflected by an improvement in the score of the BPRS item guilt feelings persists until at least 28 days after the last administration of 5-MeO-DMT or a pharmaceutically acceptable salt thereof.
142. 5-MeO-DMT or a pharmaceutically acceptable salt thereof for use as in aspects 113 or 114, wherein the patient suffers from negative thinking and an improvement in negative thinking is reflected by a reduction in the Clinical Global Impression-Severity (CGI-S) score about 2 hours after the last administration of 5-MeO-DMT or a pharmaceutically acceptable salt thereof.
143. 5-MeO-DMT or a pharmaceutically acceptable salt thereof for use as in aspects 113, 114 or 142, wherein the patient suffers from negative thinking and an improvement in negative thinking is reflected by a reduction in the Clinical Global Impression-Severity (CGI-S) score on day 1, for instance, about 24 hours after the last administration of 5-MeO-DMT or a pharmaceutically acceptable salt thereof.
144. 5-MeO-DMT or a pharmaceutically acceptable salt thereof for use as in aspects 113, 114, 142 or 143, wherein the patient suffers from negative thinking and an improvement in negative thinking is reflected by a reduction in the Clinical Global Impression-Severity (CGI-S) score on day 7 after the last administration of 5-MeO-DMT or a pharmaceutically acceptable salt thereof.
145. 5-MeO-DMT or a pharmaceutically acceptable salt thereof for use as in aspects 113, 114 or 142 to 144, wherein the patient suffers from negative thinking and an improvement in negative thinking is reflected by a reduction in the Clinical Global Impression-Severity (CGI-S) score on day 14 after the last administration of 5-MeO-DMT or a pharmaceutically acceptable salt thereof.
146. 5-MeO-DMT or a pharmaceutically acceptable salt thereof for use as in aspects 113, 114 or 142 to 145, wherein the patient suffers from negative thinking and an improvement in negative thinking is reflected by a reduction in the Clinical Global Impression-Severity (CGI-S) score on day 28 after the last administration of 5-MeO-DMT or a pharmaceutically acceptable salt thereof.
147. 5-MeO-DMT or a pharmaceutically acceptable salt thereof for use as in aspects 1 to 112, wherein the patient suffers from negative thinking and an improvement in negative thinking, as reflected by a reduction in the Clinical Global Impression-Severity (CGI-S) score, occurs not later than about 2 hours after the last administration of 5-MeO-DMT or a pharmaceutically acceptable salt thereof.
148. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 6 days after the last administration of 5-MeO-DMT or a pharmaceutically acceptable salt thereof.
149. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 14 days after the last administration of 5-MeO-DMT or a pharmaceutically acceptable salt thereof.
150. 5-MeO-DMT or a pharmaceutically acceptable salt thereof for use as in aspect 147, wherein the improvement in negative thinking, as reflected by a reduction in the Clinical Global Impression-Severity (CGI-S) score, persists until at least 28 days after the last administration of 5-MeO-DMT or a pharmaceutically acceptable salt thereof.
151. 5-MeO-DMT or a pharmaceutically acceptable salt thereof for use as in aspects 1 to 150, wherein the patient suffers from anxiety and the treatment reduces or eliminates the anxiety.
152. 5-MeO-DMT or a pharmaceutically acceptable salt thereof for use as in aspect 151, wherein the reduction or elimination of anxiety is reflected by an improvement at least in the score of the BDRS item anxiety 2 hours after the last administration of 5-MeO-DMT or a pharmaceutically acceptable salt thereof.
153. 5-MeO-DMT or a pharmaceutically acceptable salt thereof for use as in aspects 151 or 152, wherein the reduction or elimination of anxiety is reflected by an improvement at least in the score of the BDRS item anxiety on day 1, for instance, about 24 hours after the last administration of 5-MeO-DMT or a pharmaceutically acceptable salt thereof.
154. 5-MeO-DMT or a pharmaceutically acceptable salt thereof for use as in aspects 151 to 153, wherein the reduction or elimination of anxiety is reflected by an improvement at least in the score of the BDRS item anxiety on day 7 after the last administration of 5-MeO-DMT or a pharmaceutically acceptable salt thereof.
155. 5-MeO-DMT or a pharmaceutically acceptable salt thereof for use as in aspects 151 to 154, wherein the reduction or elimination of anxiety is reflected by an improvement at least in the score of the BDRS item anxiety on day 14 after the last administration of 5-MeO-DMT or a pharmaceutically acceptable salt thereof.
156. 5-MeO-DMT or a pharmaceutically acceptable salt thereof for use as in aspects 151 to 155, wherein the reduction or elimination of anxiety is reflected by an improvement at least in the score of the BDRS item anxiety on day 28 after the last administration of 5-MeO-DMT or a pharmaceutically acceptable salt thereof.
157. 5-MeO-DMT or a pharmaceutically acceptable salt thereof for use as in aspect 151, wherein the reduction or elimination of anxiety as reflected by an improvement in the score of the BDRS item anxiety occurs not later than about 2 hours after the last administration of 5-MeO-DMT or a pharmaceutically acceptable salt thereof.
158. 5-MeO-DMT or a pharmaceutically acceptable salt thereof for use as in aspect 157, wherein the reduction or elimination of anxiety as reflected by an improvement in the score of the BDRS item anxiety persists until at least 6 days after the last administration of 5-MeO-DMT or a pharmaceutically acceptable salt thereof.
159. 5-MeO-DMT or a pharmaceutically acceptable salt thereof for use as in aspect 157, wherein the reduction or elimination of anxiety as reflected by an improvement in the score of the BDRS item anxiety persists until at least 14 days after the last administration of 5-MeO-DMT or a pharmaceutically acceptable salt thereof.
160. 5-MeO-DMT or a pharmaceutically acceptable salt thereof for use as in aspect 157, wherein the reduction or elimination of anxiety as reflected by an improvement in the score of the BDRS item anxiety persists until at least 28 days after the last administration of 5-MeO-DMT or a pharmaceutically acceptable salt thereof.
161. 5-MeO-DMT or a pharmaceutically acceptable salt thereof for use as in aspect 151, wherein the reduction or elimination of anxiety is reflected by an improvement at least in the score of the BPRS item anxiety about 2 hours after the last administration of 5-MeO-DMT or a pharmaceutically acceptable salt thereof.
162. 5-MeO-DMT or a pharmaceutically acceptable salt thereof for use as in aspects 151 or 161, wherein the reduction or elimination of anxiety is reflected by an improvement at least in the score of the BPRS item anxiety on day 1 after the last administration of 5-MeO-DMT or a pharmaceutically acceptable salt thereof.
163. 5-MeO-DMT or a pharmaceutically acceptable salt thereof for use as in aspects 151, 161 or 162, wherein the reduction or elimination of anxiety is reflected by an improvement at least in the score of the BPRS item anxiety on day 7 after the last administration of 5-MeO-DMT or a pharmaceutically acceptable salt thereof.
164. 5-MeO-DMT or a pharmaceutically acceptable salt thereof for use as in aspects 151 or 161 to 163, wherein the reduction or elimination of anxiety is reflected by an improvement at least in the score of the BPRS item anxiety on day 14 after the last administration of 5-MeO-DMT or a pharmaceutically acceptable salt thereof.
165. 5-MeO-DMT or a pharmaceutically acceptable salt thereof for use as in aspects 151 or 161 to 164, wherein the reduction or elimination of anxiety is reflected by an improvement at least in the score of the BPRS item anxiety on day 28 after the last administration of 5-MeO-DMT or a pharmaceutically acceptable salt thereof.
166. 5-MeO-DMT or a pharmaceutically acceptable salt thereof for use as in aspect 151, wherein the reduction or elimination of anxiety as reflected by an improvement in the score of the BPRS item anxiety occurs not later than about 2 hours after the last administration of 5-MeO-DMT or a pharmaceutically acceptable salt thereof.
167. 5-MeO-DMT or a pharmaceutically acceptable salt thereof for use as in aspect 166, wherein the reduction or elimination of anxiety as reflected by an improvement in the score of the BPRS item anxiety persists until at least 6 days after the last administration of 5-MeO-DMT or a pharmaceutically acceptable salt thereof.
168. 5-MeO-DMT or a pharmaceutically acceptable salt thereof for use as in aspect 166, wherein the reduction or elimination of anxiety as reflected by an improvement in the score of the BPRS item anxiety persists until at least 14 days after the last administration of 5-MeO-DMT or a pharmaceutically acceptable salt thereof.
169. 5-MeO-DMT or a pharmaceutically acceptable salt thereof for use as in aspect 166, wherein the reduction or elimination of anxiety as reflected by an improvement in the score of the BPRS item anxiety persists until at least 28 days after the last administration of 5-MeO-DMT or a pharmaceutically acceptable salt thereof.
170. 5-MeO-DMT or a pharmaceutically acceptable salt thereof for use as in aspect 151, wherein the patient suffers from anxiety and an improvement in anxiety is reflected by a reduction in the Clinical Global Impression-Severity (CGI-S) score about 2 hours after the last administration of 5-MeO-DMT or a pharmaceutically acceptable salt thereof.
171. 5-MeO-DMT or a pharmaceutically acceptable salt thereof for use as in aspects 151 or 170, wherein the patient suffers from anxiety and an improvement in anxiety is reflected by a reduction in the Clinical Global Impression-Severity (CGI-S) score on day 1, for instance, about 24 hours after the last administration of 5-MeO-DMT or a pharmaceutically acceptable salt thereof.
172. 5-MeO-DMT or a pharmaceutically acceptable salt thereof for use as in aspects 151, 170 or 171, wherein the patient suffers from anxiety and an improvement in anxiety is reflected by a reduction in the Clinical Global Impression-Severity (CGI-S) score on day 7 after the last administration of 5-MeO-DMT or a pharmaceutically acceptable salt thereof.
173. 5-MeO-DMT or a pharmaceutically acceptable salt thereof for use as in aspects 151 or 170 to 172, wherein the patient suffers from anxiety and an improvement in anxiety is reflected by a reduction in the Clinical Global Impression-Severity (CGI-S) score on day 14 after the last administration of 5-MeO-DMT or a pharmaceutically acceptable salt thereof.
174. 5-MeO-DMT or a pharmaceutically acceptable salt thereof for use as in aspects 151 or 170 to 173, wherein the patient suffers from anxiety and an improvement in anxiety is reflected by a reduction in the Clinical Global Impression-Severity (CGI-S) score on day 28 after the last administration of 5-MeO-DMT or a pharmaceutically acceptable salt thereof.
175. 5-MeO-DMT or a pharmaceutically acceptable salt thereof for use as in aspects 1 to 150, wherein the patient suffers from anxiety and an improvement in anxiety, as reflected by a reduction in the Clinical Global Impression-Severity (CGI-S) score, occurs not later than about 2 hours after the last administration of 5-MeO-DMT or a pharmaceutically acceptable salt thereof.
176. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 6 days after the last administration of 5-MeO-DMT or a pharmaceutically acceptable salt thereof.
177. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 14 days after the last administration of 5-MeO-DMT or a pharmaceutically acceptable salt thereof.
178. 5-MeO-DMT or a pharmaceutically acceptable salt thereof for use as in aspect 175, wherein the improvement in anxiety, as reflected by a reduction in the Clinical Global Impression-Severity (CGI-S) score, persists until at least 28 days after the last administration of 5-MeO-DMT or a pharmaceutically acceptable salt thereof.
179. 5-MeO-DMT or a pharmaceutically acceptable salt thereof for use as in aspects 1 to 178, wherein the patient suffers from cognitive dysfunction and the treatment reduces or eliminates the cognitive dysfunction.
180. 5-MeO-DMT or a pharmaceutically acceptable salt thereof for use as in aspect 179, wherein the reduction or elimination of cognitive dysfunction is reflected by an improvement at least in the score of the BDRS item impaired concentration and memory about 2 hours after the last administration of 5-MeO-DMT or a pharmaceutically acceptable salt thereof.
181. 5-MeO-DMT or a pharmaceutically acceptable salt thereof for use as in aspects 179 or 180, wherein the reduction or elimination of cognitive dysfunction is reflected by an improvement at least in the score of the BDRS item impaired concentration and memory on day 1, for instance, about 24 hours after the last administration of 5-MeO-DMT or a pharmaceutically acceptable salt thereof.
182. 5-MeO-DMT or a pharmaceutically acceptable salt thereof for use as in aspects 179 to 181, wherein the reduction or elimination of cognitive dysfunction is reflected by an improvement at least in the score of the BDRS item impaired concentration and memory on day 7 after the last administration of 5-MeO-DMT or a pharmaceutically acceptable salt thereof.
183. 5-MeO-DMT or a pharmaceutically acceptable salt thereof for use as in aspects 179 to 182, wherein the reduction or elimination of cognitive dysfunction is reflected by an improvement at least in the score of the BDRS item impaired concentration and memory on day 14 after the last administration of 5-MeO-DMT or a pharmaceutically acceptable salt thereof.
184. 5-MeO-DMT or a pharmaceutically acceptable salt thereof for use as in aspects 179 to 183, wherein the reduction or elimination of cognitive dysfunction is reflected by an improvement at least in the score of the BDRS item impaired concentration and memory on day 28 after the last administration of 5-MeO-DMT or a pharmaceutically acceptable salt thereof.
185. 5-MeO-DMT or a pharmaceutically acceptable salt thereof for use as in aspect 179, wherein the reduction or elimination of cognitive dysfunction as reflected by an improvement in the score of the BDRS item impaired concentration and memory occurs not later than about 2 hours after the last administration of 5-MeO-DMT or a pharmaceutically acceptable salt thereof.
186. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 6 days after the last administration of 5-MeO-DMT or a pharmaceutically acceptable salt thereof.
187. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 14 days after the last administration of 5-MeO-DMT or a pharmaceutically acceptable salt thereof.
188. 5-MeO-DMT or a pharmaceutically acceptable salt thereof for use as in aspect 185, wherein the reduction or elimination of cognitive dysfunction as reflected by an improvement in the score of the BDRS item impaired concentration and memory persists until at least 28 days after the last administration of 5-MeO-DMT or a pharmaceutically acceptable salt thereof.
189. 5-MeO-DMT or a pharmaceutically acceptable salt thereof for use as in aspect 179, wherein the reduction or elimination of cognitive dysfunction is reflected by an improvement at least in the score of the MADRS item concentration difficulties about 2 hours after the last administration of 5-MeO-DMT or a pharmaceutically acceptable salt thereof.
190. 5-MeO-DMT or a pharmaceutically acceptable salt thereof for use as in aspects 179 or 189, wherein the reduction or elimination of cognitive dysfunction is reflected by an improvement at least in the score of the MADRS item concentration difficulties on day 1, for instance, about 24 hours after the last administration of 5-MeO-DMT or a pharmaceutically acceptable salt thereof.
191. 5-MeO-DMT or a pharmaceutically acceptable salt thereof for use as in aspects 179, 189 or 190, wherein the reduction or elimination of cognitive dysfunction is reflected by an improvement at least in the score of the MADRS item concentration difficulties on day 7 after the last administration of 5-MeO-DMT or a pharmaceutically acceptable salt thereof.
192. 5-MeO-DMT or a pharmaceutically acceptable salt thereof for use as in aspects 179 or 189 to 191, wherein the reduction or elimination of cognitive dysfunction is reflected by an improvement at least in the score of the MADRS item concentration difficulties on day 14 after the last administration of 5-MeO-DMT or a pharmaceutically acceptable salt thereof.
193. 5-MeO-DMT or a pharmaceutically acceptable salt thereof for use as in aspects 179 or 189 to 192, wherein the reduction or elimination of cognitive dysfunction is reflected by an improvement at least in the score of the MADRS item concentration difficulties on day 28 after the last administration of 5-MeO-DMT or a pharmaceutically acceptable salt thereof.
194. 5-MeO-DMT or a pharmaceutically acceptable salt thereof for use as in aspect 179, wherein the reduction or elimination of cognitive dysfunction as reflected by an improvement in the score of the MADRS item concentration difficulties occurs not later than about 2 hours after the last administration of 5-MeO-DMT or a pharmaceutically acceptable salt thereof.
195. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 6 days after the last administration of 5-MeO-DMT or a pharmaceutically acceptable salt thereof.
196. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 14 days after the last administration of 5-MeO-DMT or a pharmaceutically acceptable salt thereof.
197. 5-MeO-DMT or a pharmaceutically acceptable salt thereof for use as in aspect 194, wherein the reduction or elimination of cognitive dysfunction as reflected by an improvement in the score of the MADRS item concentration difficulties persists until at least 28 days after the last administration of 5-MeO-DMT or a pharmaceutically acceptable salt thereof.
198. 5-MeO-DMT or a pharmaceutically acceptable salt thereof for use as in aspect 179, wherein the patient suffers from cognitive dysfunction and an improvement in cognitive dysfunction is reflected by a reduction in the Clinical Global Impression-Severity (CGI-S) score about 2 hours after the last administration of 5-MeO-DMT or a pharmaceutically acceptable salt thereof.
199. 5-MeO-DMT or a pharmaceutically acceptable salt thereof for use as in aspects 179 or 198, wherein the patient suffers from cognitive dysfunction and an improvement in cognitive dysfunction is reflected by a reduction in the Clinical Global Impression-Severity (CGI-S) score on day 1, for instance, about 24 hours after the last administration of 5-MeO-DMT or a pharmaceutically acceptable salt thereof.
200. 5-MeO-DMT or a pharmaceutically acceptable salt thereof for use as in aspects 179, 198 or 199, wherein the patient suffers from cognitive dysfunction and an improvement in cognitive dysfunction is reflected by a reduction in the Clinical Global Impression
Severity (CGI-S) score on day 7 after the last administration of 5-MeO-DMT or a pharmaceutically acceptable salt thereof.
201. 5-MeO-DMT or a pharmaceutically acceptable salt thereof for use as in aspects 179 or 198 to 200, wherein the patient suffers from cognitive dysfunction and an improvement in cognitive dysfunction is reflected by a reduction in the Clinical Global Impression-Severity (CGI-S) score on day 14 after the last administration of 5-MeO-DMT or a pharmaceutically acceptable salt thereof.
202. 5-MeO-DMT or a pharmaceutically acceptable salt thereof for use as in aspects 179 or 198 to 201, wherein the patient suffers from cognitive dysfunction and an improvement in cognitive dysfunction is reflected by a reduction in the Clinical Global Impression-Severity (CGI-S) score on day 28 after the last administration of 5-MeO-DMT or a pharmaceutically acceptable salt thereof.
203. 5-MeO-DMT or a pharmaceutically acceptable salt thereof for use as in aspects 1 to 178, wherein the patient suffers from cognitive dysfunction and an improvement in cognitive dysfunction, as reflected by a reduction in the Clinical Global Impression-Severity (CGI-S) score, occurs not later than about 2 hours after the last administration of 5-MeO-DMT or a pharmaceutically acceptable salt thereof.
204. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 6 days after the last administration of 5-MeO-DMT or a pharmaceutically acceptable salt thereof.
205. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 14 days after the last administration of 5-MeO-DMT or a pharmaceutically acceptable salt thereof.
206. 5-MeO-DMT or a pharmaceutically acceptable salt thereof for use as in aspect 203, wherein the improvement in cognitive dysfunction, as reflected by a reduction in the Clinical Global Impression-Severity (CGI-S) score, persists until at least 28 days after the last administration of 5-MeO-DMT or a pharmaceutically acceptable salt thereof.
207. 5-MeO-DMT or a pharmaceutically acceptable salt thereof for use as in aspects 1 to 206, wherein the patient suffers from social/emotional withdrawal or detachment and the treatment reduces or eliminates the social/emotional withdrawal or detachment.
208. 5-MeO-DMT or a pharmaceutically acceptable salt thereof for use as in aspect 207, wherein the treatment reduces or eliminates at least one of anhedonia, emotional withdrawal and affective flattening.
209. 5-MeO-DMT or a pharmaceutically acceptable salt thereof for use as in aspects 207 or 208, wherein the reduction or elimination of social/emotional withdrawal or detachment is reflected by an improvement at least in the scores of the BDRS items anhedonia, emotional withdrawal and/or affective flattening about 2 hours after the last administration of 5-MeO-DMT or a pharmaceutically acceptable salt thereof.
210. 5-MeO-DMT or a pharmaceutically acceptable salt thereof for use as in aspects 207 to 209, wherein the reduction or elimination of social/emotional withdrawal or detachment is reflected by an improvement at least in the scores of the BDRS items anhedonia, emotional withdrawal and/or affective flattening on day 1, for instance, about 24 hours after the last administration of 5-MeO-DMT or a pharmaceutically acceptable salt thereof.
211. 5-MeO-DMT or a pharmaceutically acceptable salt thereof for use as in aspects 207 to 210, wherein the reduction or elimination of social/emotional withdrawal or detachment is reflected by an improvement at least in the score of the BDRS items anhedonia, emotional withdrawal and/or affective flattening on day 7 after the last administration of 5-MeO-DMT or a pharmaceutically acceptable salt thereof.
212. 5-MeO-DMT or a pharmaceutically acceptable salt thereof for use as in aspects 207 to 211, wherein the reduction or elimination of social/emotional withdrawal or detachment is reflected by an improvement at least in the scores of the BDRS items anhedonia, emotional withdrawal and/or affective flattening on day 14 after the last administration of 5-MeO-DMT or a pharmaceutically acceptable salt thereof.
213. 5-MeO-DMT or a pharmaceutically acceptable salt thereof for use as in aspects 207 to 212, wherein the reduction or elimination of social/emotional withdrawal or detachment is reflected by an improvement at least in the scores of the BDRS items anhedonia, emotional withdrawal and/or affective flattening on day 28 after the last administration of 5-MeO-DMT or a pharmaceutically acceptable salt thereof.
214. 5-MeO-DMT or a pharmaceutically acceptable salt thereof for use as in aspects 207 or 208, wherein the reduction or elimination of social/emotional withdrawal or detachment as reflected by an improvement in the scores of the BDRS items anhedonia, emotional withdrawal and/or affective flattening occurs not later than about 2 hours after the last administration of 5-MeO-DMT or a pharmaceutically acceptable salt thereof.
215. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 6 days after the last administration of 5-MeO-DMT or a pharmaceutically acceptable salt thereof.
216. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 14 days after the last administration of 5-MeO-DMT or a pharmaceutically acceptable salt thereof.
217. 5-MeO-DMT or a pharmaceutically acceptable salt thereof for use as in aspect 214, wherein the reduction or elimination of social/emotional withdrawal or detachment as reflected by an improvement in the scores of the BDRS items anhedonia, emotional withdrawal and/or affective flattening persists until at least 28 days after the last administration of 5-MeO-DMT or a pharmaceutically acceptable salt thereof.
218. 5-MeO-DMT or a pharmaceutically acceptable salt thereof for use as in aspects 207 or 208, wherein the reduction or elimination of social/emotional withdrawal or detachment is reflected by an improvement at least in the score of the MADRS item inability to feel about 2 hours after the last administration of 5-MeO-DMT or a pharmaceutically acceptable salt thereof.
219. 5-MeO-DMT or a pharmaceutically acceptable salt thereof for use as in aspects 207, 208 or 218, wherein the reduction or elimination of social/emotional withdrawal or detachment is reflected by an improvement at least in the score of the MADRS item inability to feel on day 1, for instance, about 24 hours days after the last administration of 5-MeO-DMT or a pharmaceutically acceptable salt thereof.
220. 5-MeO-DMT or a pharmaceutically acceptable salt thereof for use as in aspects 207, 208, 218 or 219, wherein the reduction or elimination of social/emotional withdrawal or detachment is reflected by an improvement at least in the score of the MADRS item inability to feel on day 7 days after the last administration of 5-MeO-DMT or a pharmaceutically acceptable salt thereof.
221. 5-MeO-DMT or a pharmaceutically acceptable salt thereof for use as in aspects 207, 208 or 218 to 220, wherein the reduction or elimination of social/emotional withdrawal or detachment is reflected by an improvement at least in the score of the MADRS item inability to feel on day 14 days after the last administration of 5-MeO-DMT or a pharmaceutically acceptable salt thereof.
222. 5-MeO-DMT or a pharmaceutically acceptable salt thereof for use as in aspects 207, 208 or 218 to 221, wherein the reduction or elimination of social/emotional withdrawal or detachment is reflected by an improvement at least in the score of the MADRS item inability to feel on day 28 after the last administration of 5-MeO-DMT or a pharmaceutically acceptable salt thereof.
223. 5-MeO-DMT or a pharmaceutically acceptable salt thereof for use as in aspects 207 or 208, wherein the reduction or elimination of social/emotional withdrawal or detachment as reflected by an improvement in the score of the MADRS item inability to feel occurs not later than about 2 hours after the last administration of 5-MeO-DMT or a pharmaceutically acceptable salt thereof.
224. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 6 days after the last administration of 5-MeO-DMT or a pharmaceutically acceptable salt thereof.
225. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 14 days after the last administration of 5-MeO-DMT or a pharmaceutically acceptable salt thereof.
226. 5-MeO-DMT or a pharmaceutically acceptable salt thereof for use as in aspect 223, wherein the reduction or elimination of social/emotional withdrawal or detachment as reflected by an improvement in the score of the MADRS item inability to feel persists until at least 28 days after the last administration of 5-MeO-DMT or a pharmaceutically acceptable salt thereof.
227. 5-MeO-DMT or a pharmaceutically acceptable salt thereof for use as in aspects 207 or 208, wherein the reduction or elimination of social/emotional withdrawal or detachment is reflected by an improvement at least in the score of the BPRS item emotional withdrawal about 2 hours after the last administration of 5-MeO-DMT or a pharmaceutically acceptable salt thereof.
228. 5-MeO-DMT or a pharmaceutically acceptable salt thereof for use as in aspects 207, 208 or 227, wherein the reduction or elimination of social/emotional withdrawal or detachment is reflected by an improvement at least in the score of the BPRS item emotional withdrawal on day 1, for instance, about 24 hours after the last administration of 5-MeO-DMT or a pharmaceutically acceptable salt thereof.
229. 5-MeO-DMT or a pharmaceutically acceptable salt thereof for use as in aspects 207, 208, 227 or 228, wherein the reduction or elimination of social/emotional withdrawal or detachment is reflected by an improvement at least in the score of the BPRS item emotional withdrawal on day 7 after the last administration of 5-MeO-DMT or a pharmaceutically acceptable salt thereof.
230. 5-MeO-DMT or a pharmaceutically acceptable salt thereof for use as in aspects 207, 208 or 227 to 229, wherein the reduction or elimination of social/emotional withdrawal or detachment is reflected by an improvement at least in the score of the BPRS item emotional withdrawal on day 14 after the last administration of 5-MeO-DMT or a pharmaceutically acceptable salt thereof.
231. 5-MeO-DMT or a pharmaceutically acceptable salt thereof for use as in aspects 207, 208 or 227 to 230, wherein the reduction or elimination of social/emotional withdrawal or detachment is reflected by an improvement at least in the score of the BPRS item emotional withdrawal on day 28 after the last administration of 5-MeO-DMT or a pharmaceutically acceptable salt thereof.
232. 5-MeO-DMT or a pharmaceutically acceptable salt thereof for use as in aspects 207 or 208, wherein the reduction or elimination of social/emotional withdrawal or detachment as reflected by an improvement in the score of the BPRS item emotional withdrawal occurs not later than about 2 hours after the last administration of 5-MeO-DMT or a pharmaceutically acceptable salt thereof.
233. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 6 days after the last administration of 5-MeO-DMT or a pharmaceutically acceptable salt thereof.
234. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 14 days after the last administration of 5-MeO-DMT or a pharmaceutically acceptable salt thereof.
235. 5-MeO-DMT or a pharmaceutically acceptable salt thereof for use as in aspect 232, wherein the reduction or elimination of social/emotional withdrawal or detachment as reflected by an improvement in the score of the BPRS item emotional withdrawal persists until at least 28 days after the last administration of 5-MeO-DMT or a pharmaceutically acceptable salt thereof.
236. 5-MeO-DMT or a pharmaceutically acceptable salt thereof for use as in aspects 207 or 208, wherein the reduction or elimination of social/emotional withdrawal or detachment is reflected by an improvement at least in the score of the BPRS item blunted affect about 2 hours after the last administration of 5-MeO-DMT or a pharmaceutically acceptable salt thereof.
237. 5-MeO-DMT or a pharmaceutically acceptable salt thereof for use as in aspects 207, 208 or 236, wherein the reduction or elimination of social/emotional withdrawal or detachment is reflected by an improvement at least in the score of the BPRS item blunted affect on day 1, for instance, about 24 hours after the last administration of 5-MeO-DMT or a pharmaceutically acceptable salt thereof.
238. 5-MeO-DMT or a pharmaceutically acceptable salt thereof for use as in aspects 207, 208, 236 or 237, wherein the reduction or elimination of social/emotional withdrawal or detachment is reflected by an improvement at least in the score of the BPRS item blunted affect on day 7 after the last administration of 5-MeO-DMT or a pharmaceutically acceptable salt thereof.
239. 5-MeO-DMT or a pharmaceutically acceptable salt thereof for use as in aspects 207, 208 or 236 to 238, wherein the reduction or elimination of social/emotional withdrawal or detachment is reflected by an improvement at least in the score of the BPRS item blunted affect on day 14 after the last administration of 5-MeO-DMT or a pharmaceutically acceptable salt thereof.
240. 5-MeO-DMT or a pharmaceutically acceptable salt thereof for use as in aspects 207, 208 or 236 to 239, wherein the reduction or elimination of social/emotional withdrawal or detachment is reflected by an improvement at least in the score of the BPRS item blunted affect on day 28 after the last administration of 5-MeO-DMT or a pharmaceutically acceptable salt thereof.
241. 5-MeO-DMT or a pharmaceutically acceptable salt thereof for use as in aspects 207 or 208, wherein the reduction or elimination of social/emotional withdrawal or detachment as reflected by an improvement in the score of the BPRS item blunted affect occurs not later than about 2 hours after the last administration of 5-MeO-DMT or a pharmaceutically acceptable salt thereof.
242. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 6 days after the last administration of 5-MeO-DMT or a pharmaceutically acceptable salt thereof.
243. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 14 days after the last administration of 5-MeO-DMT or a pharmaceutically acceptable salt thereof.
244. 5-MeO-DMT or a pharmaceutically acceptable salt thereof for use as in aspect 241, wherein the reduction or elimination of social/emotional withdrawal or detachment as reflected by an improvement in the score of the BPRS item blunted affect persists until at least 28 days after the last administration of 5-MeO-DMT or a pharmaceutically acceptable salt thereof.
245. 5-MeO-DMT or a pharmaceutically acceptable salt thereof for use as in aspects 207 or 208, wherein the patient suffers from social/emotional withdrawal or detachment and an improvement in social/emotional withdrawal or detachment is reflected by a reduction in the Clinical Global Impression-Severity (CGI-S) score about 2 hours after the last administration of 5-MeO-DMT or a pharmaceutically acceptable salt thereof.
246. 5-MeO-DMT or a pharmaceutically acceptable salt thereof for use as in aspects 207, 208 or 245, wherein the patient suffers from social/emotional withdrawal or detachment and an improvement in social/emotional withdrawal or detachment is reflected by a reduction in the Clinical Global Impression-Severity (CGI-S) score on day 1, for instance, about 24 hours after the last administration of 5-MeO-DMT or a pharmaceutically acceptable salt thereof.
247. 5-MeO-DMT or a pharmaceutically acceptable salt thereof for use as in aspects 207, 208, 245 or 246, wherein the patient suffers from social/emotional withdrawal or detachment and an improvement in social/emotional withdrawal or detachment is reflected by a reduction in the Clinical Global Impression-Severity (CGI-S) score on day 7 after the last administration of 5-MeO-DMT or a pharmaceutically acceptable salt thereof.
248. 5-MeO-DMT or a pharmaceutically acceptable salt thereof for use as in aspects 207, 208 or 245 to 247, wherein the patient suffers from social/emotional withdrawal or detachment and an improvement in social/emotional withdrawal or detachment is reflected by a reduction in the Clinical Global Impression-Severity (CGI-S) score on day 14 after the last administration of 5-MeO-DMT or a pharmaceutically acceptable salt thereof.
249. 5-MeO-DMT or a pharmaceutically acceptable salt thereof for use as in aspects 207, 208 or 245 to 248, wherein the patient suffers from social/emotional withdrawal or detachment and an improvement in social/emotional withdrawal or detachment is reflected by a reduction in the Clinical Global Impression-Severity (CGI-S) score on day 28 after the last administration of 5-MeO-DMT or a pharmaceutically acceptable salt thereof.
250. 5-MeO-DMT or a pharmaceutically acceptable salt thereof for use as in aspects 1 to 206, wherein the patient suffers from social/emotional withdrawal or detachment and an improvement at least in social/emotional withdrawal or detachment, as reflected by a reduction in the Clinical Global Impression-Severity (CGI-S) score, occurs not later than about 2 hours after the last administration of 5-MeO-DMT or a pharmaceutically acceptable salt thereof.
251. 5-MeO-DMT or a pharmaceutically acceptable salt thereof for use as in aspect 250, wherein the improvement in social/emotional withdrawal or detachment, as reflected by a reduction at least in the Clinical Global Impression-Severity (CGI-S) score, persists until at least 6 days after the last administration of 5-MeO-DMT or a pharmaceutically acceptable salt thereof.
252. 5-MeO-DMT or a pharmaceutically acceptable salt thereof for use as in aspect 251, wherein the improvement in social/emotional withdrawal or detachment, as reflected by a reduction at least in the Clinical Global Impression-Severity (CGI-S) score, persists until at least 14 days after the last administration of 5-MeO-DMT or a pharmaceutically acceptable salt thereof.
253. 5-MeO-DMT or a pharmaceutically acceptable salt thereof for use as in aspect 251, wherein the improvement in social/emotional withdrawal or detachment, as reflected by a reduction at least in the Clinical Global Impression-Severity (CGI-S) score, persists until at least 28 days after the last administration of 5-MeO-DMT or a pharmaceutically acceptable salt thereof.
254. 5-MeO-DMT or a pharmaceutically acceptable salt thereof for use as in aspects 1 to 253, wherein the treatment reduces or eliminates suicidal ideation.
255. 5-MeO-DMT or a pharmaceutically acceptable salt thereof for use as in aspect 254, wherein the reduction or elimination of suicidal ideation is reflected by an improvement at least in the score of the BDRS item suicidal ideation about 2 hours after the last administration of 5-MeO-DMT or a pharmaceutically acceptable salt thereof.
256. 5-MeO-DMT or a pharmaceutically acceptable salt thereof for use as in aspects 254 or 255, wherein the reduction or elimination of suicidal ideation is reflected by an improvement at least in the score of the BDRS item suicidal ideation on day 1, for instance, about 24 hours after the last administration of 5-MeO-DMT or a pharmaceutically acceptable salt thereof.
257. 5-MeO-DMT or a pharmaceutically acceptable salt thereof for use as in aspects 254 to 256, wherein the reduction or elimination of suicidal ideation is reflected by an improvement at least in the score of the BDRS item suicidal ideation on day 7 after the last administration of 5-MeO-DMT or a pharmaceutically acceptable salt thereof.
258. 5-MeO-DMT or a pharmaceutically acceptable salt thereof for use as in aspects 254 to 257, wherein the reduction or elimination of suicidal ideation is reflected by an improvement at least in the score of the BDRS item suicidal ideation on day 14 after the last administration of 5-MeO-DMT or a pharmaceutically acceptable salt thereof.
259. 5-MeO-DMT or a pharmaceutically acceptable salt thereof for use as in aspects 254 to 258, wherein the reduction or elimination of suicidal ideation is reflected by an improvement at least in the score of the BDRS item suicidal ideation on day 28 after the last administration of 5-MeO-DMT or a pharmaceutically acceptable salt thereof.
260. 5-MeO-DMT or a pharmaceutically acceptable salt thereof for use as in aspect 254, wherein the reduction or elimination of suicidal ideation as reflected by an improvement in the score of the BDRS item suicidal ideation occurs not later than about 2 hours after the last administration of 5-MeO-DMT or a pharmaceutically acceptable salt thereof.
261. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 6 days after the last administration of 5-MeO-DMT or a pharmaceutically acceptable salt thereof.
262. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 14 days after the last administration of 5-MeO-DMT or a pharmaceutically acceptable salt thereof.
263. 5-MeO-DMT or a pharmaceutically acceptable salt thereof for use as in aspect 260, wherein the reduction or elimination of suicidal ideation as reflected by an improvement in the score of the BDRS item suicidal ideation persists until at least 28 days after the last administration of 5-MeO-DMT or a pharmaceutically acceptable salt thereof.
264. 5-MeO-DMT or a pharmaceutically acceptable salt thereof for use as in aspect 254, wherein the reduction or elimination of suicidal ideation is reflected by an improvement at least in the score of the MADRS item suicidal thoughts about 2 hours after the last administration of 5-MeO-DMT or a pharmaceutically acceptable salt thereof.
265. 5-MeO-DMT or a pharmaceutically acceptable salt thereof for use as in aspects 254 or 264, wherein the reduction or elimination of suicidal ideation is reflected by an improvement at least in the score of the MADRS item suicidal thoughts on day 1, for instance, about 24 hours after the last administration of 5-MeO-DMT or a pharmaceutically acceptable salt thereof.
266. 5-MeO-DMT or a pharmaceutically acceptable salt thereof for use as in aspects 254, 264 or 265, wherein the reduction or elimination of suicidal ideation is reflected by an improvement at least in the score of the MADRS item suicidal thoughts on day 7 after the last administration of 5-MeO-DMT or a pharmaceutically acceptable salt thereof.
267. 5-MeO-DMT or a pharmaceutically acceptable salt thereof for use as in aspects 254 or 264 to 266, wherein the reduction or elimination of suicidal ideation is reflected by an improvement at least in the score of the MADRS item suicidal thoughts on day 14 after the last administration of 5-MeO-DMT or a pharmaceutically acceptable salt thereof.
268. 5-MeO-DMT or a pharmaceutically acceptable salt thereof for use as in aspects 254 or 264 to 267, wherein the reduction or elimination of suicidal ideation is reflected by an improvement at least in the score of the MADRS item suicidal thoughts on day 28 after the last administration of 5-MeO-DMT or a pharmaceutically acceptable salt thereof.
269. 5-MeO-DMT or a pharmaceutically acceptable salt thereof for use as in aspect 254, wherein the reduction or elimination of suicidal ideation as reflected by an improvement in the score of the MADRS item suicidal thoughts occurs not later than about 2 hours after the last administration of 5-MeO-DMT or a pharmaceutically acceptable salt thereof.
270. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 6 days after the last administration of 5-MeO-DMT or a pharmaceutically acceptable salt thereof.
271. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 14 days after the last administration of 5-MeO-DMT or a pharmaceutically acceptable salt thereof.
272. 5-MeO-DMT or a pharmaceutically acceptable salt thereof for use as in aspect 269, wherein the reduction or elimination of suicidal ideation as reflected by an improvement in the score of the MADRS item suicidal thoughts persists until at least 28 days after the last administration of 5-MeO-DMT or a pharmaceutically acceptable salt thereof.
273. 5-MeO-DMT or a pharmaceutically acceptable salt thereof for use as in aspect 254, wherein the patient suffers from suicidal ideation and an improvement in suicidal ideation is reflected by a reduction in the Clinical Global Impression-Severity (CGI-S) score about 2 hours after the last administration of 5-MeO-DMT or a pharmaceutically acceptable salt thereof.
274. 5-MeO-DMT or a pharmaceutically acceptable salt thereof for use as in aspects 254 or 273, wherein the patient suffers from suicidal ideation and an improvement in suicidal ideation is reflected by a reduction in the Clinical Global Impression-Severity (CGI-S) score on day 1, for instance, about 24 hours after the last administration of 5-MeO-DMT or a pharmaceutically acceptable salt thereof.
275. 5-MeO-DMT or a pharmaceutically acceptable salt thereof for use as in aspects 254, 273 or 274, wherein the patient suffers from suicidal ideation and an improvement in suicidal ideation is reflected by a reduction in the Clinical Global Impression-Severity (CGI-S) score on day 7 after the last administration of 5-MeO-DMT or a pharmaceutically acceptable salt thereof.
276. 5-MeO-DMT or a pharmaceutically acceptable salt thereof for use as in aspects 254 or 273 to 275, wherein the patient suffers from suicidal ideation and an improvement in suicidal ideation is reflected by a reduction in the Clinical Global Impression-Severity (CGI-S) score on day 14 after the last administration of 5-MeO-DMT or a pharmaceutically acceptable salt thereof.
277. 5-MeO-DMT or a pharmaceutically acceptable salt thereof for use as in aspects 254 or 273 to 276, wherein the patient suffers from suicidal ideation and an improvement in suicidal ideation is reflected by a reduction in the Clinical Global Impression-Severity (CGI-S) score on day 28 after the last administration of 5-MeO-DMT or a pharmaceutically acceptable salt thereof.
278. 5-MeO-DMT or a pharmaceutically acceptable salt thereof for use as in aspects 1 to 253, wherein the patient suffers from suicidal ideation and an improvement in suicidal ideation, as reflected by a reduction in the Clinical Global Impression-Severity (CGI-S) score, occurs not later than about 2 hours after the last administration of 5-MeO-DMT or a pharmaceutically acceptable salt thereof.
279. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 6 days after the last administration of 5-MeO-DMT or a pharmaceutically acceptable salt thereof.
280. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 14 days after the last administration of 5-MeO-DMT or a pharmaceutically acceptable salt thereof.
281. 5-MeO-DMT or a pharmaceutically acceptable salt thereof for use as in aspect 278, wherein the improvement in suicidal ideation, as reflected by a reduction in the Clinical Global Impression-Severity (CGI-S) score, persists until at least 28 days after the last administration of 5-MeO-DMT or a pharmaceutically acceptable salt thereof.
282. 5-MeO-DMT or a pharmaceutically acceptable salt thereof for use as in aspects 1 to 281, wherein the treatment reduces or eliminates at least one of psychotic symptoms; irritability; lability; increased motor drive; increased speech; agitation.

### EXAMPLES

The following Examples are listed to aid understanding of the invention and are not intended and should not be construed to limit in any way the invention set forth in the claims which follow thereafter.

### Example 1—5-MeO-DMT Aerosol Generation and Administration

Step 1: A stock solution of 5-MeO-DMT free base in 100% ethanol is prepared in a volumetric flask, so that the target dosage of 5-MeO-DMT free base to be administered via inhalation to the volunteer or patient is contained in a solution volume of 200 μl. Typical target dosages are from 1 mg to 25 mg 5-MeO-DMT. E.g. for a target dosage of 18 mg 5-MeO-DMT, 90 mg of 5-MeO-DMT will be dissolved in 100% ethanol for a final solution volume of 1 ml. Aliquots of the stock solution can then be stored in vials until further use.

Step 2:200 μl of the solution is transferred to a dosing capsule containing the drip pad (Storz & Bickel, Germany), and then the dosing capsule is closed with its lid.

Step 3: The dosing capsule filled with the 5-MeO-DMT ethanol solution is transferred to the filling chamber of a first Volcano Medic Vaporizer, which has been pre-heated with the temperature set at 55° C. Then the airflow of the vaporizer is switched on for 60 seconds at the pre-set rate of about 12 l/min. The heated air will flow through the dosing capsule, allowing the ethanol to evaporate, with the target dosage of 5-MeO-DMT being left in the capsule, as a thin layer covering the stainless-steel wire mesh. Accurate preparation of the dosing capsule can be confirmed by demonstrating that the final weight increase of the capsule compared to the weight of the empty capsule is about equal to the target dosage of 5-MeO-DMT.

Step 4: The prepared dosing capsule is removed from the filling chamber. It is then transferred to the filling chamber of a second Volcano Medic Vaporizer, which has been preheated with the temperature set at 210° C. and the airflow on for at least 5 minutes and then turned off immediately prior to transfer. An inhalation balloon with a valve (Storz & Bickel, Germany) is mounted on the socket of the filling chamber, the filling chamber is closed tightly and immediately afterwards the airflow is switched on for exactly 15 seconds at the pre-set flow rate of about 12 l/min, and then turned off. This will allow the full dose of 5-MeO-DMT to aerosolize and be distributed in approximately 3 liters of air in the inhalation balloon. Accurate aerosolization of the 5-MeO-DMT can be confirmed by demonstrating that the capsule weight has returned to about its initial weight.

Step 5: The balloon is then disconnected from the filling chamber, which automatically closes the valve. After attachment of the mouthpiece to the balloon, the aerosol is ready for immediate administration to the volunteer or patient.

Step 6: To prepare for the administration, the patient is asked to initially perform 1-2 deep inhalations with full exhalations, ending this sequence with a deep exhalation. Then, with the mouthpiece firmly held against the lips, the full and complete volume of the inhalation balloon is inhaled in one inhalation, holding the breath for 10 (±2.5) seconds, followed by a normal exhalation. After completing the inhalation procedure, the patient will be instructed to lie down.

Further details regarding the administration of 5-MeO-DMT by inhalation are disclosed in Example 1 of WO 2020/169850 A1, the contents of which is incorporated herein by reference.

### Example 2—Preparation of 5-MeO-DMT in High Purity

5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to 12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC as described in WO 2020/169850 A1. The purity was found to be 99.74% area.

The results from the analysis further indicate that the level of individual impurities was below 0.10% area. Solvent analysis of sample indicated an MTBE level of 17 ppm.

### Example 3—Preparation of 5-MeO-DMT Hydrobromide Salt

5-MeO-DMT HBr was prepared on a 100 mg scale.

5-MeO-DMT free base was combined with isopropyl acetate (10 vols), and the resulting solution of 5-MeO-DMT was heated to 50° C. HBr was charged (1M in ethanol, 1 eq) in one single aliquot. The mixture was held at temperature and equilibrated for 3 hours.

After 1 hour, a suspension had formed. The suspension was finally cooled to room temperature and equilibrated for 18 hours. Solids were isolated by filtration and dried in vacuo at 40° C. for 18 hours.

An off-white crystalline material was obtained.

The salt has a melting point of 174° C. and is characterized by an X-ray diffraction pattern comprising peaks at 14.5° 2·±0.2° 2·; 16.7° 2·±0.2° 2·; 17.0° 2·±0.2° 2·; 20.6° 2·±0.2° 2·; 20.7° 2·±0.2° 2·; 21.4° 2·±0.2° 2·; 24.2°2·±0.2°2·; 24.8° 2·±0.2°2·; 25.3°2·±0.2° 2·; 27.4° 2·±0.2° 2·; measured using Cu K· radiation.

### Example 4—Determination of Inhibition Constants for Central 5-HT1A and 5-HT2A Receptors in Post-Mortem Human Brain Membrane Preparations

In this study, the affinity of three psychedelic test compounds (psilocin, DMT and 5-MeO-DMT) for 5-HT1A and 5-HT2A receptors in post-mortem human brain tissue from the hippocampus and frontal cortex, respectively, was determined using the technique of radioligand binding.

Human brain samples were obtained from the Edinburgh Sudden Death Brain Bank. All donors were sudden deaths with no prior history of coma, psychiatric or neurological disorders and under the age of 65 with a post-mortem interval of less than or equal to 72 hours.

### Binding to 5-HT1A Receptors in Post-Mortem Human Hippocampus

Hippocampus was homogenised in ice-cold 0.25 M sucrose (1:30 w/v) using a motor driven Teflon pestle (12 strokes at 120 rpm). Myelin and cell debris were removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold membrane preparation buffer, (1:100 w/v) using a tight-fitting glass/Teflon homogeniser (12 strokes, 800 rpm) and centrifuged at 20,500 g for 10 minutes. The pellet was resuspended in ice-cold membrane preparation buffer and incubated at 37° C. for 10 minutes before being centrifuged at 20,500 g for 10 minutes. The pellet was resuspended and centrifuged a final time to wash the tissue (20,500×g for 10 mins). The resulting pellet was then resuspended in ice-cold assay buffer at a tissue concentration equivalent to 3.125 mg wet weight of tissue/ml. All centrifugations were carried out at 4° C. The membrane preparation buffer consisted of 50 mM Tris-HCl, pH 7.7, 4 mM CaCl₂) and 0.1% ascorbic acid. The assay buffer consisted of 50 mM Tris, pH 7.7, 4 mM CaCl₂), 0.1% ascorbic acid and 10 UM Pargyline.

For saturation binding analysis, hippocampal membranes (400 μl; equivalent 1.25 mg wet weight tissue/tube) was incubated with 50 μl of 0.075-9.6 nM [³H] 8-OH-DPAT and either 50 μl of assay buffer (total binding) or 50 μl of 1 M WAY 100635 (non-specific binding) at 25° C. for 30 minutes. The wash buffer consisted of 50 mM Tris, pH 7.7.

In a displacement assay, hippocampal membranes (400 μl; equivalent 1.25 mg wet weight tissue/tube) were incubated with 50 μl of 0.6 nM [³H] 8-OH-DPAT and either 50 μl of assay buffer (total binding) or 50 μl of 1·M WAY 100635 (non-specific binding) or 50 μl of one of the test compounds in one of ten concentrations between 1 and 10000 nM at 25° C. for 30 minutes.

Membrane bound radioactivity was recovered by filtration under vacuum through Skatron 11731 filters, pre-soaked in 0.5% polyethylenimine (PEI) using a Skatron cell harvester. Filters were rapidly washed with ice-cold wash buffer (wash setting 0,9,9) and radioactivity determined by liquid scintillation counting (1 ml Packard MV Gold scintillator).

The concentration of compound required to inhibit 50% of specific binding (IC₅₀) and the Hill Slope were calculated by using non-linear regression. The Kᵢ was calculated using the one-site binding model allowing for ligand depletion.

### Binding to 5-HT2A Receptors in Post-Mortem Human Frontal Cortex

Frontal cortex was homogenised in ice-cold 0.25 M sucrose (1:30 w/v) using a motor driven Teflon pestle (12 strokes at 120 rpm). Myelin and cell debris was removed by centrifugation at 1,000 g for 10 minutes. The supernatant was stored on ice and the pellet re-homogenised in 0.25 M sucrose (1:15 w/v) and centrifuged at 750 g for 10 minutes. The supernatants were combined and diluted in ice-cold 50 mM Tris-HCl assay buffer, pH 7.4 (1:100 w/v), homogenised using a tight-fitting glass/Teflon homogeniser (12 strokes, 800 rpm) and centrifuged at 20,500 g for 10 minutes. The pellet was centrifuged a further two times to wash the tissue (20,500×g for 10 mins). The resulting pellet was then resuspended in ice-cold 50 mM Tris-HCl assay buffer, pH 7.4 at a tissue concentration equivalent to 10 mg wet weight of tissue/ml. All centrifugations were carried out at 4° C.

For saturation binding analysis, frontal cortical membranes (400 μl; equivalent to 4 mg wet weight of tissue/tube) were incubated with 50 μl of 0.00625-0.8 nM [³H] MDL-100,907 and either 50 μl of assay buffer or 50 μl of 10. M ketanserin (non-specific binding) at 25° C. for 60 minutes. The assay and wash buffer consisted of 50 mM Tris-HCl buffer pH 7.4.

In a displacement assay, frontal cortical membranes (400 μl; equivalent 4 mg wet weight tissue/tube) was incubated with 50 μl of 0.1 nM [³H] MDL-100,907 and either 50 μl of assay buffer (total binding) or 50 μl of 10·M ketanserin (non-specific binding) or 50 μl of one of the test compounds in one of ten concentrations between 1 and 10000 nM at 25° C. for 60 minutes.

Membrane bound radioactivity was recovered and determined as above. Data analysis was also as above.

### Results

The dissociation constant (K_{d} value) of [³H] 8-OH-DPAT for 5-HT1A receptors in hippocampal membranes from post-mortem human brain tissue was determined for each of the three donors. The dissociation constants (K_{d} values) obtained were 0.51, 0.28 and 0.52 nM, respectively.

Mean inhibition constants (Kᵢ values) for psilocin, DMT and 5-MeO-DMT were 48, 38 and 1.80 nM (mean n=3), respectively. All compounds gave Hill slopes approximating to unity, suggesting a one-site binding model.

The dissociation constant (K_{d} values) of [³H] MDL-100,907 for 5-HT2A receptors in frontal cortical membranes from post-mortem human brain tissue was determined for each of the three donors. The dissociation constants (K_{d} values) obtained were 0.11, 0.08 and 0.08 nM, respectively.

Mean inhibition constants (Kᵢ values) for psilocin, DMT and 5-MeO-DMT were 37, 117 and 122 nM (mean n=3), respectively. All compounds gave Hill slopes approximating to unity, suggesting a one-site binding model.

The selectivity ratio of psilocin, DMT and 5-MeO-DMT for 5-HT2A over 5-HT1A receptors was 0.78, 3.1 and 68, respectively.

### Example 5—Clinical Trial in Patients Suffering from TRD

A Phase 1/2 clinical trial of 5-MeO-DMT, administered via inhalation as described herein, in patients with treatment-resistant major depressive disorder (TRD) has been completed. This trial was designed in two parts. Part A was an open-label, single-arm, single-dose Phase 1 trial with two dose levels (12 mg (n=4) and 18 mg (n=4)). Part B was an open-label, single-arm Phase 2 trial applying an individualized dosing regimen with intra-patient dose escalation with 5-MeO-DMT. Patients (n=8) received at least one and up to three doses of 5-MeO-DMT (6 mg, 12 mg and 18 mg) in a single day, with higher doses only being administered if a peak experience was not achieved at the previously administered dose. The primary endpoint of Part A was to assess the safety and tolerability of single dosing of 5-MeO-DMT in patients with TRD. The primary endpoint of Part B was to assess the effects on the severity of depression, as assessed by the proportion of patients in remission on day seven after dosing, defined as a MADRS total score of less than or equal to 10.

In Part A, 3 of 4 patients in both groups (12 mg and 18 mg) experienced at least one ADR, all of which were mild and resolved spontaneously. No SAEs were reported.

Two of four patients (50%) in the 12 mg group and one of four patients (25%) in the 18 mg group had a MADRS remission on day seven after dosing, and one further patient (25%) in the 18 mg group had a MADRS clinical response on day seven after dosing. The mean MADRS change from baseline at day seven was −21.0 (−65%) in the 12 mg group and −12.8 (−41%) in the 18 mg group.

In Part B, 7 of 8 patients (87.5%) experienced at least one ADR. All ADRs resolved spontaneously. No SAEs were reported.

The primary endpoint was met with seven of eight patients (87.5%) achieving a MADRS remission on day seven (p<0.0001). The mean MADRS change from baseline at day seven was 24.4 (76%).

No clinically significant changes were observed in either Part A or Part B in any of the safety laboratory analyses, vital signs, psychiatric safety assessments or measures of cognitive function.

Results are summarized in the tables below.

TABLE 1-A
Scores recorded against relevant MADRS and BPRS items for patients as-signed to
intra-day individualised dosing regimen (IDR). The item scores represent the sum
of the individual patient scores for all patients (n = 8) in the IDR group.
Assessment 2 hours (MADRS) or 3 hours (BPRS) after administration of the last dose.

| | | Baseline | 2 / 3 | Improvement | Percentage |
|---|---|---|---|---|---|
| Scale item | Scale | IDR | hours | 2 / 3 hours | improvement |
| Concentration | MADRS | 30 | 11 | 19 | 63.33% |
| Difficulties | | | | | |
| Lassitude | MADRS | 27 | 10 | 17 | 62.96% |
| Inability to Feel | MADRS | 36 | 12 | 24 | 66.67% |
| Inner Tension | MADRS | 26 | 11 | 15 | 57.69% |
| Pessimistic Thoughts | MADRS | 28 | 7 | 21 | 75.00% |
| Suicidal Thoughts | MADRS | 11 | 3 | 8 | 72.72% |
| Guilt Feelings | BPRS | 34 | 14 | 20 | 58.82% |
| Tension | BPRS | 16 | 11 | 5 | 31.25% |
| Anxiety | BPRS | 37 | 19 | 18 | 48.65% |
| Emotional Withdrawal | BPRS | 13 | 8 | 5 | 38.46% |
| Blunted Affect | BPRS | 15 | 11 | 4 | 26.67% |

TABLE 1-B
Scores recorded against relevant MADRS and BPRS items for patients
as-signed to intra-day individualised dosing regimen (IDR). The
item scores represent the sum of the individual patient scores
for all patients (n = 8) in the IDR group. Assessment on day 1.

| | | Baseline | | Improvement | Percentage |
|---|---|---|---|---|---|
| Scale item | Scale | IDR | Day 1 | at Day 1 | improvement |
| Reduced Sleep | MADRS | 25 | 12 | 13 | 52.00% |
| Concentration | MADRS | 30 | 1 | 29 | 96.67% |
| Difficulties | | | | | |
| Lassitude | MADRS | 27 | 5 | 22 | 81.48% |
| Inability to Feel | MADRS | 36 | 2 | 34 | 94.44% |
| Inner Tension | MADRS | 26 | 6 | 20 | 76.92% |
| Pessimistic Thoughts | MADRS | 28 | 4 | 24 | 85.71% |
| Suicidal Thoughts | MADRS | 11 | 1 | 10 | 90.91% |
| Guilt Feelings | BPRS | 34 | 11 | 23 | 67.65% |
| Tension | BPRS | 16 | 11 | 5 | 31.25% |
| Anxiety | BPRS | 37 | 16 | 21 | 56.76% |
| Emotional Withdrawal | BPRS | 13 | 8 | 5 | 38.46% |
| Blunted Affect | BPRS | 15 | 8 | 7 | 46.67% |

TABLE 1-C
Scores recorded against relevant MADRS and BPRS items for patients
as-signed to intra-day individualised dosing regimen (IDR). The
item scores represent the sum of the individual patient scores
for all patients (n = 8) in the IDR group. Assessment on day 7.

| | | Baseline | | Improvement | Percentage |
|---|---|---|---|---|---|
| Scale item | Scale | IDR | Day 7 | at Day 7 | improvement |
| Reduced Sleep | MADRS | 25 | 9 | 16 | 64.00% |
| Concentration | MADRS | 30 | 9 | 21 | 70.00% |
| Difficulties | | | | | |
| Lassitude | MADRS | 27 | 3 | 24 | 88.89% |
| Inability to Feel | MADRS | 36 | 6 | 30 | 83.33% |
| Inner Tension | MADRS | 26 | 12 | 14 | 53.85% |
| Pessimistic Thoughts | MADRS | 28 | 3 | 25 | 89.29% |
| Suicidal Thoughts | MADRS | 11 | 3 | 8 | 72.73% |
| Guilt Feelings | BPRS | 34 | 10 | 24 | 70.59% |
| Tension | BPRS | 16 | 10 | 6 | 37.50% |
| Anxiety | BPRS | 37 | 17 | 20 | 54.05% |
| Emotional Withdrawal | BPRS | 13 | 8 | 5 | 38.46% |
| Blunted Affect | BPRS | 15 | 8 | 7 | 46.67% |

TABLE 2-A
Scores recorded against relevant MADRS and BPRS items for patients as-signed
to 12mg dosing regimen. The item scores represent the sum of the individual
patient scores for all patients (n = 4) in the 12 mg group. Assessment
2 hours (MADRS) or 3 hours (BPRS) after administration of the dose.

| | | | | Improvement | |
|---|---|---|---|---|---|
| | | Baseline | 2 / 3 | after 2 / 3 | Percentage |
| Scale item | Scale | 12 mg | hours | hours | improvement |
| Concentration | MADRS | 16 | 7 | 9 | 56.25% |
| Difficulties | | | | | |
| Lassitude | MADRS | 16 | 10 | 6 | 37.50% |
| Inability to Feel | MADRS | 16 | 9 | 7 | 43.75% |
| Inner Tension | MADRS | 13 | 2 | 11 | 84.62% |
| Pessimistic Thoughts | MADRS | 16 | 8 | 8 | 50.00% |
| Suicidal Thoughts | MADRS | 8 | 3 | 5 | 62.50% |
| Guilt Feelings | BPRS | 18 | 9 | 9 | 50.00% |
| Tension | BPRS | 14 | 9 | 5 | 35.71% |
| Anxiety | BPRS | 25 | 11 | 14 | 56.00% |
| Emotional Withdrawal | BPRS | 13 | 11 | 2 | 15.38% |
| Blunted Affect | BPRS | 11 | 8 | 3 | 27.27% |

TABLE 2-B
Scores recorded against relevant MADRS and BPRS items for patients as-signed
to 12mg dosing regimen. The item scores represent the sum of the individual
patient scores for all patients (n = 4) in the 12 mg group. Assessment on day 1.

| | | Baseline | | Improvement | Percentage |
|---|---|---|---|---|---|
| Scale item | Scale | 12 mg | Day 1 | at Day 1 | improvement |
| Reduced Sleep | MADRS | 12 | 10 | 2 | 16.67% |
| Concentration | MADRS | 16 | 2 | 14 | 87.50% |
| Difficulties | | | | | |
| Lassitude | MADRS | 16 | 0 | 16 | 100.00% |
| Inability to Feel | MADRS | 16 | 1 | 15 | 93.75% |
| Inner Tension | MADRS | 13 | 3 | 10 | 76.92% |
| Pessimistic Thoughts | MADRS | 16 | 7 | 9 | 56.25% |
| Suicidal Thoughts | MADRS | 8 | 5 | 3 | 37.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 8 | 5 | 38.46% |
| Blunted Affect | BPRS | 11 | 6 | 5 | 45.45% |

TABLE 2-C
Scores recorded against relevant MADRS and BPRS items for patients as-signed
to 12mg dosing regimen. The item scores represent the sum of the individual
patient scores for all patients (n = 4) in the 12 mg group. Assessment on day 7.

| | | Baseline | | Improvement | Percentage |
|---|---|---|---|---|---|
| Scale item | Scale | 12 mg | Day 7 | at Day 7 | improvement |
| Reduced Sleep | MADRS | 12 | 6 | 6 | 50.00% |
| Concentration | MADRS | 16 | 3 | 13 | 81.25% |
| Difficulties | | | | | |
| Lassitude | MADRS | 16 | 3 | 13 | 81.25% |
| Inability to Feel | MADRS | 16 | 1 | 15 | 93.75% |
| Inner Tension | MADRS | 13 | 5 | 8 | 61.54% |
| Pessimistic Thoughts | MADRS | 16 | 8 | 8 | 50.00% |
| Suicidal Thoughts | MADRS | 8 | 7 | 1 | 12.50% |
| Guilt Feelings | BPRS | 18 | 5 | 13 | 72.22% |
| Tension | BPRS | 14 | 6 | 8 | 57.14% |
| Anxiety | BPRS | 25 | 6 | 19 | 76.00% |
| Emotional Withdrawal | BPRS | 13 | 6 | 7 | 53.85% |
| Blunted Affect | BPRS | 11 | 5 | 6 | 54.55% |

### Example 6—Assessment of the Pharmacokinetics of 5-MeO-DMT and Bufotenine

In order to investigate the pharmacokinetic properties of 5-MeO-DMT, three groups of 8 subjects each were formed. Subjects were administered a single dose of 6 mg; 12 mg or 18 mg 5-MeO-DMT via inhalation. Blood samples were obtained at 1; 2; 4; 7; 10; 15; 20; 30; 45 min and 1; 1.5; 2; 3; 4 hours after administration.

5-MeO-DMT concentrations were determined using LC-MS/MS. PK parameters were generated by algebraic analysis of the concentration versus time plots for each individual. The analysis was carried out using the software Phoenix WinNonlin 6.3.

Median Cmax values obtained for the three groups were 11.85 ng/ml (6 mg group), 22.90 ng/ml (12 mg group) and 38.45 ng/ml (18 mg group).

Table 3 below shows median percentage plasma concentrations relative to Cmax as determined for the time points indicated.

| time | | Median percentage plasma concentration relative to Cmax | | | | | | | | | | | | | |
|---|---|---|---|---|---|---|---|---|---|---|---|---|---|---|---|
| (min) | | 1 | 2 | 4 | 7 | 10 | 15 | 20 | 30 | 45 | 60 | 90 | 120 | 180 | 240 |
| 6 | mg | 96 | 98 | 83 | 56 | 40 | 22 | 16 | 10 | 8 | 4 | 2 | 1 | 0 | N.A. |
| 12 | mg | 100 | 79 | 59 | 30 | 22 | 12 | 8 | 5 | 3 | 2 | 0 | 0 | 0 | 0 |
| 18 | mg | 100 | 79 | 59 | 30 | 22 | 12 | 8 | 5 | 3 | 2 | 0 | 0 | 0 | 0 |

Pharmacokinetic measurements were also carried out for dosing schemes relying on uptitration. Substantially similar results were obtained.

Blood concentrations were also determined for the 5-MeO-DMT metabolite bufotenine. Only in few samples, concentrations were above the lower level of quantification (LLOQ) (25 pg/ml). From 15 min onwards, the bufotenine concentration was always below the LLOQ.

Substantially similar observations were made when subjects receiving an uptitration scheme were included.

### Example 7—Toxicological Testing of 5-MeO-DMT

5-MeO-DMT did not induce mutation in four histidine-requiring bacterial strains (TA98, TA100, TA1535 and TA1537) of Salmonella typhimurium, and one tryptophan-requiring strain (WP2 uvrA pKM101) of Escherichia coli. These conditions included treatments at concentrations up to 5000 μg/plate (the maximum recommended concentration according to current regulatory guidelines), in the absence and in the presence of a rat liver metabolic activation system (S-9).

### Example 8—Clinical Trial of 5-MeO-DMT Administered Via Inhalation to Patients with Postpartum Depression

The single-arm, open-label clinical trial will involve 15 adult female patients with clinically diagnosed postpartum depression (PPD).

The patients will receive a single-day individualized 5-MeO-DMT dosing regimen via inhalation after vaporization.

More in particular, the patients will receive up to three doses of 5-MeO-DMT on Day 0:6 mg, 12 mg, and 18 mg.
1. All patients will receive an initial dose of 6 mg 5-MeO-DMT.
2. The second dose (12 mg) will only be administered if:
   a. A peak experience (total score of ·75) has not been achieved following the 6 mg dose, and
   b. The 6 mg dose was safe and well-tolerated according to the investigator,
   c. Any psychoactive effects (PsE) of the prior dose have subsided, and
   d. Pre-dose vital parameters and forced expiratory volume in one second (FEV1) are in normal range, or if outside of the normal range, are not clinically significant according to the investigator.
3. Similarly, a third dose (18 mg) will only be administered if:
   a. A peak experience (total score of ·75) has not been achieved following the 12 mg dose, and
   b. The 12 mg dose was safe and well-tolerated according to the investigator, and
   c. Any PsE of the prior dose have subsided, and
   d. Pre-dose vital parameters and forced expiratory volume in one second (FEV1) are in normal range, or if outside of the normal range, are not clinically significant according to the investigator.

The patients will be assessed for a peak psychedelic experience (based on a patient-scored visual analogue scale, the PE scale), sedation, and other endpoints after dosing. Follow-up visits are planned for Day 1, and Day 7 after the dosing day.

The following criteria must be met by all patients considered for clinical trial participation:
1. Is female and in the age range between 18 and 45 years (inclusive) at screening.
2. Has a body mass index (BMI) in the range of 18.5 and 35 kg/m² (inclusive) at screening.
3. Meets the trial criteria for PPD as assessed by a trial psychiatrist or registered psychologist:
   a. Diagnosis of Major Depressive Disorder without psychotic features, confirmed by the Mini-International Neuropsychiatric Interview (MINI), with peri-partum onset that began no earlier than gestation and no later than the first 4 weeks postpartum.
   b. Has a Montgomery-Åsberg Depression Rating Scale (MADRS) total score of equal to or greater than 28 at screening and pre-dose on Day 0.
6. Must have either ceased lactating at screening; or, if still lactating or actively breast feeding at screening, must agree to temporarily cease breastfeeding from just prior to receiving study drug on Day 0 through 24 hours post last dose, and to pump and discard all breastmilk during those 24 hours as needed, but need to include a pump/discard at 2.5 hours post last dose and 24 hours post last dose prior to reinitiating breastfeeding.
4. Must agree to remain completely abstinent (complete avoidance of heterosexual intercourse) or use a highly effective (failure rate <1%), medically accepted contraceptive method for 30 days prior to dosing and for 90 days after 5-MeO-DMT dosing. Patients must have a negative pregnancy test at screening and on the pre-test day (Day-1).
5. Is willing to delay start of other antidepressant or anxiety medication until after the end of the trial at Day 7 and agrees to keep any psychotherapy unchanged during the trial.

A potential patient who meets any of the following key exclusion criteria will be excluded from participation in this trial:
1. Has, based on history, psychiatric assessment, and evaluation of the MINI, a current or prior diagnosis of bipolar disorder, a manic or hypomanic episode, a psychotic disorder, Major Depressive Disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed bipolar disorder, psychotic disorder or other mood disorder (including MDD) with psychotic features.
3. Is in the judgement of a trial psychiatrist or registered psychologist, at significant risk for suicide based on history, psychiatric assessment, and evaluation of suicidal ideation and suicidal behaviour based on the Columbia-Suicide Severity Rating Scale (C-SSRS).
4. Has taken anti-depressive medication within 14 days or 5 half-lives (whichever is longer) prior to dosing (exception: within the last 5 weeks in the case of fluoxetine).
5. Has taken any other medication with monoamine oxidase inhibitor (MAOI) activity within 14 days or 5 half-lives (whichever is longer) prior to dosing.
6. Has previously experienced a significant adverse reaction to a hallucinogenic or psychedelic drug (e.g., psilocybin, Psilocybe spp. mushrooms, 5-MeO-DMT, DMT, ayahuasca, LSD, mescaline) according to the investigator's judgment.
7. Has known allergies or hypersensitivity or any other contraindication to 5-MeO-DMT.
8. Has any current or past clinically significant condition (e.g., severe infection, pulmonary disease, uncontrolled hypertension, new onset of hypertensive disorders of pregnancy during pregnancy or in the postnatal period (e.g., gestational hypertension, pre-eclampsia-eclampsia, superimposed pre-eclampsia), uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that renders the patient unsuitable for the trial according to the investigator's judgment.
9. Takes any medication or other substance that renders the patient unsuitable for the trial according to the investigator's judgment.
10. Has a clinically significant abnormality in physical examination, vital signs, ECG, or clinical laboratory parameters which renders the patient unsuitable for the trial according to the investigator's judgment.
11. Patient who has a positive pregnancy test at screening or on the pre-test day (Day-1), is pregnant, or plans to become pregnant during the course of the trial and up to 90 days after 5-MeO-DMT dosing.
12. Patients with DSM-5 drug or alcohol use disorder within 6 months prior to screening.

The primary objective of the trial is to determine the onset and 7-day durability of anti-depressive effects of a single-day individualized dosing regimen of 6 mg, 12 mg and 18 mg of 5-MeO-DMT in adult, female patients with PPD.

Secondary objectives are to determine the anti-depressive effects; the anti-anxiety effects; the effects on maternal behavior; the safety and tolerability; the intensity and duration of psychoactive effects (PsE); the impact on cognitive outcome of a single-day individualized dosing regimen of 6 mg, 12 mg and 18 mg of 5-MeO-DMT in adult, female patients with PPD.

An exploratory objective is to determine in breastmilk, blood and urine the amount of 5-MeO-DMT and metabolites, bufotenine and 5-methoxyindole-3-acetic acid (5-MIAA), measured by LC/MS/MS (metabolite identification screening may be performed, as required), following dose administration of a single-day IDR of 6 mg, 12 mg and 18 mg of 5-MeO-DMT in adult, female patients with PPD.

The primary endpoint of the study is the evaluation of the anti-depressive effects of 5-MeO-DMT by the change from baseline in MADRS assessed at Day 7.

Secondary endpoints include the anti-depressive effects of 5-MeO-DMT evaluated by
The anti-depressive effects of 5-MeO-DMT evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in Clinical Global Impression-Severity scale (CGI-S) 2 hours after final study drug dosing on Day 0, and at Day 1 and Day 7;
Effects on maternal behaviour as assessed by the change from baseline in the Barkin Index of Maternal Functioning (BIMF) total and sub-scale scores to Day 7;
Exposure of 5-MeO-DMT and bufotenine in breastmilk obtained at the pre-test day (Day-1), 1 hour after last study drug dosing, at discharge, in the evening of Day 0, and on Day 1 and on Day 7;
Exposure of 5-MeO-DMT and bufotenine in blood obtained at the pre-test day (Day-1), 1 hour after last study drug dosing, at discharge, on Day 1 and on Day 7;
The safety and tolerability of 5-MeO-DMT evaluated by:
   Reporting of treatment-emergent adverse events (TEAEs);
   Clinically significant changes from baseline in ECG, vital signs, safety laboratory assessments, peak flow respirometry;
   Assessment of sedation (Modified Observer's Assessment of Alertness and Sedation scale [MOAA/S]) following each dose (when the PsE has subsided and 60 minutes after each study drug dosing) and as part of the discharge evaluation on Day 0;
   Change from baseline in Clinician Administered Dissociative States Scale (CADSS) assessed as part of the discharge evaluation on Day 0 and at Day 1 and Day 7;
   Change from baseline in Brief Psychiatric Rating Scale (BPRS) assessed as part of the discharge evaluation on Day 0, and at Day 1 and Day 7;
   Change from baseline in C-SSRS assessed as part of the discharge evaluation on Day 0, and at Day 1 and Day 7;
   Change from baseline in YMRS assessed as part of the discharge evaluation on Day 0, and at Day 1 and Day 7;
The PsE experienced by the patients as reported 30 to 60 minutes after each dosing, when the PsE has subsided:
   PsE assessment using the peak experience (PE) scale to assess the achievement of a PE (PE scale total score ·75);
   Challenging Experience Questionnaire (CEQ);
   Mystical Experience Questionnaire (MEQ-30);
Duration of the PsE defined as the time from study drug dosing to the time when the PsE have subsided (investigator- and patient-scored), completed 30 to 60 minutes after each dosing;

One patient with postpartum depression diagnosed by a psychiatrist has, so far, been recruited into the clinical trial. Diagnosis was Major Depressive Disorder without psychotic features, confirmed by the Mini-International Neuropsychiatric Interview (MINI) (v7.0.2), with peri-partum onset that began no earlier than gestation and no later than the first 4 weeks postpartum. The patient was diagnosed with postpartum depression after giving birth to her third child. The patient completed all planned visit days. The inhalation procedure was adequately performed by the patient and was well tolerated with no inhalation-related adverse events.

### Results

Except for a temporary, clinically non-relevant increase in heart rate and blood pressure shortly after administration of 5-MeO-DMT, no other noteworthy changes in vital parameters occurred. Assessments of ECG (at 3 hours after administration) and safety laboratory analyses (at 7 days), CADSS (at 3 hours, 1 day and 7 days) were unremarkable. The few reported adverse events (cramping left abdominal pain and headache, both on Day 0) were mild, short-lasting and resolved spontaneously by the end of the study.

With regard to the intensity of the psychedelic experience, the recorded PES score achieved upon exposure to a nominal dose of 6 mg was 17.3. This score indicated the need to proceed to the administration of a subsequent, higher dose of 12 mg, per the design of the individualised dosing regimen. The PES score achieved for this dose was 85.7 and, being ·75, indicated the occurrence of a peak psychedelic experience and the completion of the IDR for this patient.

Significantly, the patient reported a major improvement in her depressive symptoms as assessed by MADRS at the earliest assessment timepoint of 2 hours after drug administration, with the effect being maintained over time (Table 4). The patient also fulfilled standard criteria for MADRS response (at least 50% improvement from baseline) and MADRS remission (MADRS total score equal or less than 10).

TABLE 4
MADRS/BPRS scores table

| | | | 2 hr | | | | | |
|---|---|---|---|---|---|---|---|---|
| | | | (MADRS); | | | Improvement | | |
| | | Base- | Discharge | | | 2 hr; | | |
| Scale item | Scale | line | (BPRS) | Day 1 | Day 7 | Discharge | Day 1 | Day 7 |
| Reported sadness | MADRS | 4 | 0 | 0 | 0 | 4 | 4 | 4 |
| Apparent sadness | MADRS | 5 | 0 | 0 | 0 | 5 | 5 | 5 |
| Inner Tension | MADRS | 5 | 0 | 0 | 1 | 5 | 5 | 4 |
| Reduced Sleep | MADRS | 5 | 5 | 0 | 0 | 0 | 5 | 5 |
| Reduced appetite | MADRS | 0 | 0 | 0 | 0 | 0 | 0 | 0 |
| Concentration Difficulties | MADRS | 0 | 0 | 0 | 0 | 0 | 0 | 0 |
| Lassitude | MADRS | 3 | 0 | 0 | 0 | 3 | 3 | 3 |
| Inability to Feel | MADRS | 3 | 0 | 0 | 0 | 3 | 3 | 3 |
| Pessimistic Thoughts | MADRS | 4 | 0 | 0 | 0 | 4 | 4 | 4 |
| Suicidal Thoughts | MADRS | 0 | 0 | 0 | 0 | 0 | 0 | 0 |
| Total MADRS | | 29 | 5 | 0 | 1 | 24 | 29 | 28 |
| Somatic Concern | BPRS | 6 | 1 | 1 | 1 | 5 | 5 | 5 |
| Anxiety | BPRS | 7 | 1 | 1 | 1 | 6 | 6 | 6 |
| Emotional withdrawal | BPRS | 5 | 1 | 1 | 1 | 4 | 4 | 4 |
| Conceptual disorganization | BPRS | 1 | 1 | 1 | 1 | 0 | 0 | 0 |
| Guilt feelings | BPRS | 6 | 1 | 1 | 1 | 5 | 5 | 5 |
| Tension | BPRS | 6 | 1 | 1 | 1 | 5 | 5 | 5 |
| Mannerisms and Posturing | BPRS | 1 | 1 | 1 | 1 | 0 | 0 | 0 |
| Grandiosity | BPRS | 1 | 1 | 1 | 1 | 0 | 0 | 0 |
| Depressive Mood | BPRS | 6 | 1 | 1 | 1 | 5 | 5 | 5 |
| Hostility | BPRS | 1 | 1 | 1 | 1 | 0 | 0 | 0 |
| Suspiciousness | BPRS | 1 | 1 | 1 | 1 | 0 | 0 | 0 |
| Hallucinatory behaviour | BPRS | 1 | 1 | 1 | 1 | 0 | 0 | 0 |
| Motor retardation | BPRS | 1 | 1 | 1 | 1 | 0 | 0 | 0 |
| Uncooperativeness | BPRS | 1 | 1 | 1 | 1 | 0 | 0 | 0 |
| Unusual Thought Content | BPRS | 1 | 1 | 1 | 1 | 0 | 0 | 0 |
| Blunted affect | BPRS | 1 | 1 | 1 | 1 | 0 | 0 | 0 |
| Excitement | BPRS | 2 | 1 | 1 | 1 | 1 | 1 | 1 |
| Disorientation | BPRS | 1 | 1 | 1 | 1 | 0 | 0 | 0 |

Significant improvements were noted for several MADRS items in particular. The items are outlined in Table 4. While the patient's baseline scores for some items reflected absence of the symptom (reduced appetite, concentration difficulties, suicidal thoughts), items with scores reflecting severe symptoms (e.g., reduced sleep, inner tension) saw remarkable improvement.

Similarly, improvements were seen in several BPRS items, including Somatic Concerns, Anxiety, Emotional withdrawal, Guilt feelings and Tension.

### Summary and Conclusions

A. An individualised dosing regimen of 6 mg 5-MeO-DMT, followed by 12 mg 5-MeO-DMT administered via inhalation was well tolerated and induced an astonishing and very significant clinical response in a patient formally diagnosed with postpartum depression.
B. The clinical response occurs rapidly within 2 hours after 5-MeO-DMT administration. Such rapid onset is unusual and has not been seen with conventional classes of antidepressants, including tricyclic antidepressants, monoamine oxidase inhibitors, selective serotonin-reuptake inhibitors (SSRIs), serotonin-norepinephrine reuptake inhibitors (SNRIs), and others, which generally take 4 to 6 weeks to show their effect.
C. The patient experienced a clinical remission within 2 hours after 5-MeO-DMT administration according to the IDR. This is highly superior to any approved therapy for postpartum depression, and also to all previously tested psychedelic agents.
D. A significant clinical response was sustained over the 7-day follow-up period, although 5-MeO-DMT was only given once and is no longer efficaciously present in the body during this time frame (see pharmacokinetic data above). This observation supports the superior clinical profile of 5-MeO-DMT and allows for convenient administration intervals.
E. In addition to anti-depressive effects, endpoints assessing other symptoms (such as somatic concerns, emotional withdrawal, anxiety, guilt and tension) were positively impacted, supporting the use of 5-MeO-DMT in patients with other mental diseases.

The highlighted aspects show that 5-MeO-DMT has a significantly improved efficacy profile compared to approved pharmacological therapies for postpartum depression and to all previously tested psychedelic agents, when used according to the present dosing regimen.

### Example 9—Clinical Trial of 5-MeO-DMT Administered Via Inhalation to Patients with Bipolar II Disorder

The single-arm, open-label clinical trial will involve 15 adult patients with bipolar II disorder and a current major depressive episode.

Patients who are currently taking anti-depressive medication need to discontinue or taper over time such medication.

The patients will receive a single-day individualized 5-MeO-DMT dosing regimen via inhalation after vaporization.

More in particular, the patients will receive up to three doses of 5-MeO-DMT on the administration day (Day 0): 6 mg, 12 mg, and 18 mg.
1. All patients will receive an initial dose of 6 mg 5-MeO-DMT.
2. The second dose (12 mg) will only be administered if:
   a. A peak experience (PES total score of ·75) has not been achieved following the 6 mg dose, and
   b. The 6 mg dose was safe and well-tolerated.
3. Similarly, a third dose (18 mg) will only be administered if:
   a. A peak experience (PES total score of ·75) has not been achieved following the 12 mg dose, and
   b. The 12 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day-1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, ·uncontrolled·hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day-1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 6 mg, 12 mg and 18 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 6 mg, 12 mg and 18 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via inhalation evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7.
The safety and tolerability of 5-MeO-DMT administered via inhalation evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day-1) to Day 1 and to Day 7 in the Pittsburgh Sleep Quality Index (PSQI).
The impact on cognitive outcomes as evaluated by change from the pre-test day (Day-1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 10—Clinical Trial of 5-MeO-DMT Administered Via Intravenous Injection to Patients with Bipolar II Disorder-Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 2 mg, 5 mg, and 8 mg.
1. All patients will receive an initial dose of 2 mg 5-MeO-DMT.
2. The second dose (5 mg) will only be administered if:
   a. A peak experience (PES total score of ·75) has not been achieved following the 2 mg dose, and
   b. The 2 mg dose was safe and well-tolerated.
3. Similarly, a third dose (8 mg) will only be administered if:
   a. A peak experience (PES total score of ·75) has not been achieved following the 5 mg dose, and
   b. The 5 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day-1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, ·uncontrolled·hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day-1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 2 mg, 5 mg and 8 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 2 mg, 5 mg and 8 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7.
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day-1) to Day 1 and to Day 7 in the Pittsburgh Sleep Quality Index (PSQI).
The impact on cognitive outcomes as evaluated by change from the pre-test day (Day-1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

### Example 11—Clinical Trial of 5-MeO-DMT Administered Via Intravenous Injection to Patients with Bipolar II Disorder-Prophetic Example

The clinical trial will involve adult patients with bipolar II disorder and a current major depressive episode.

Patients who are currently taking anti-depressive medication need to discontinue or taper over time such medication.

The patients will receive a single-day individualized 5-MeO-DMT dosing regimen by intravenous injection. The 5-MeO-DMT will be provided in the form of its hydrobromide salt and a formulation for intravenous injection. It is understood that the dosage amounts for 5-MeO-DMT mentioned below relate to the weight amount of the free base and the dosage amounts for the hydrobromide salt of 5-MeO-DMT can be calculated assuming that equimolar amounts are used.

More in particular, the patients will receive up to three doses of 5-MeO-DMT on the administration day (Day 0): 1 mg, 2 mg, and 3 mg.
1. All patients will receive an initial dose of 1 mg 5-MeO-DMT.
2. The second dose (2 mg) will only be administered if:
   a. A peak experience (PES total score of ·75) has not been achieved following the 1 mg dose, and
   b. The 1 mg dose was safe and well-tolerated.
3. Similarly, a third dose (3 mg) will only be administered if:
   a. A peak experience (PES total score of ·75) has not been achieved following the 2 mg dose, and
   b. The 2 mg dose was safe and well-tolerated.

The patients will be assessed for a peak psychedelic experience based on the patient-scored PES described above, sedation and other endpoints after dosing. Follow-up visits are planned for Day 1, and Day 7 after the dosing day.

Selection of patients is based on the following key inclusion criteria:
1. Understands the nature of the clinical trial and has provided signed and dated written informed consent in accordance with local regulations before the conduct of any trial-related procedures.
2. Is male or female and in the age range between 18 and 64 years (inclusive) at screening.
3. Meets the trial criteria for bipolar II disorder and is experiencing a major depressive episode, as assessed by a trial psychiatrist or registered clinical psychologist:
   a. Meets the Diagnostic and Statistical Manual of Mental Disorders (DSM-5) diagnostic criteria for bipolar II disorder with a current major depressive disorder episode confirmed by the Mini-International Neuropsychiatric Interview (MINI);
   b. Has a Montgomery-Åsberg Depression Rating Scale (MADRS) total score of equal to or greater than 24 at screening and prior to first dose on Day 0;
4. Has a Young Mania Rating Scale (YMRS) total score less than or equal to 8 at screening and prior to first dose on Day 0;
5. Agrees to keep any psychotherapy unchanged, and not initiate any new psychoactive medications during the course of the trial.
6. Female patients must be either surgically sterile (hysterectomy, tubal ligation, or bilateral oophorectomy (6 months prior to screening)) or postmenopausal with amenorrhea for the last 2 years or remain completely abstinent (complete avoidance of heterosexual intercourse) or use a highly effective (failure rate <1%) medically accepted contraceptive method, including, but not limited to, bilateral tubal ligation/occlusion, hormone contraceptives that inhibit ovulation, intrauterine device (including hormone-releasing intrauterine device/systems) for 30 days before and 90 days after 5-MeO-DMT dosing and must have a negative serum pregnancy test at screening and a negative urine pregnancy test on the pre-test day (Day-1).
7. Male patients must use prophylactic contraception (i.e., condom with spermicide or abstinence) and must not donate sperm for 30 days after 5-MeO-DMT dosing.

A potential patient who meets any of the following key exclusion criteria will be excluded from participation in this trial:
1. Has, based on history, psychiatric assessment, and evaluation of the MINI, a current or prior diagnosis of bipolar I disorder, a manic episode, a psychotic disorder, major depressive disorder (MDD) or other mood disorder with psychotic features, obsessive compulsive disorder, post-traumatic stress disorder (PTSD), autism spectrum disorder, borderline personality disorder, schizophrenia, delusional disorder, paranoid personality disorder, schizoaffective disorder, clinically significant intellectual disability, or any other psychiatric comorbidity that renders the patient unsuitable for the trial according to the investigator's judgment.
2. Has one or more first or second degree relatives with a current or previously diagnosed psychotic disorder, bipolar I disorder, or MDD with psychotic features.
3. Has significant suicide risk as defined by: (a) suicidal ideation as endorsed on items 4 or 5 on the C-SSRS within the past year, during the screening period, or at Baseline; or (b) suicidal behaviours within the past year; or (c) clinical assessment of significant suicidal risk during clinical interview; or (d) non-suicidal self-injury within the past year.
4. Has taken anti-depressive medication within 7 days or 5 half-lives (whichever is longer) prior to dosing (exception: within the last 5 weeks in the case of fluoxetine).
5. Has taken a medication with monoamine oxidase inhibitor (MAOI) activity within 14 days or 5 half-lives (whichever is longer) prior to dosing.
6. Has taken mood stabilizer therapy (e.g., lamotrigine, valproate, atypical antipsychotics) within 14 days (28 days for lithium) or 5 half-lives (whichever is longer) prior to dosing or takes mood stabilizer therapy at screening or is expected to require mood stabilizer therapy during the study (as per the investigator's judgment).
7. Has previously experienced a significant adverse reaction to a hallucinogenic or psychedelic drug according to the investigator's judgment.
8. Has known allergies or hypersensitivity or any other contraindication to 5-MeO-DMT.
9. Has any current or past clinically significant condition (e.g., severe infection, severe pulmonary disease, uncontrolled hypertension, uncontrolled diabetes, severe cardiovascular disease, severe hepatic or renal failure, severe brain disorder (including seizure disorder, stroke, dementia, degenerative neurologic diseases, meningitis, encephalitis, and head injury with loss of consciousness) that may interfere with the interpretation of the trial results, constitutes a health risk for the patient, or that otherwise renders the patient unsuitable for the trial according to the investigator's judgment.
10. Takes any medication or other substance that renders the patient unsuitable for the trial according to the investigator's judgment.
11. Has a clinically significant abnormality in physical examination, vital signs, electrocardiogram (ECG), or clinical laboratory parameters which renders the patient unsuitable for the trial according to the investigator's judgment.
12. Female patient who has a positive pregnancy test at screening or on the pre-test day (Day-1), is pregnant or lactating, or plans to become pregnant during the course of the trial and up to 30 days after 5-MeO-DMT dosing.
13. Patients with DSM-5, alcohol or substance use disorder (excluding tobacco or caffeine use disorders) within 6 months prior to screening.

The primary objective of the trial is to determine the onset and durability of anti-depressive effects of a single-day individualized dosing regimen of 1 mg, 2 mg and 3 mg of 5-MeO-DMT in patients with bipolar II disorder and a current major episode of depression. Secondary Objectives are to determine the effect on depressive symptoms and global clinical status; the safety and tolerability; the intensity and duration of psychoactive effects (PsE); the impact on sleep quality; the impact on cognitive outcomes of a single-day individualized dosing regimen of 1 mg, 2 mg and 3 mg of 5-MeO-DMT in patients with bipolar II disorder and a current major episode of depression.

The primary endpoint of the study is the evaluation of the anti-depressive effects of 5-MeO-DMT by the change from baseline in MADRS assessed at Day 7.

Secondary endpoints include:
The anti-depressive effects of 5-MeO-DMT administered via intravenous injection evaluated by:
   The proportion of patients in remission (MADRS·10) at 2 hours after the final study drug dosing on Day 0, and at Day 1 and Day 7;
   Change from baseline in MADRS assessed at 2 hours after the final study drug dosing on Day 0, and at Day 1;
   The proportion of responders (·50% reduction from baseline in MADRS total score) at 2 hours after the final study drug dosing on Day 0, and at Day 1 and Day 7;
   Change from baseline in CGI-S at 2 hours after the final study drug dosing on Day 0, and at Day 1 and Day 7.
   Change from baseline in BDRS at Day 1 and Day 7.
The safety and tolerability of 5-MeO-DMT administered via intravenous injection evaluated by:
   Reporting of treatment-emergent adverse events (TEAEs);
   Clinically significant changes from baseline in ECG, vital signs, safety laboratory assessments and spirometry assessments;
   Assessment of sedation (Modified Observer's Assessment of Alertness and Sedation scale [MOAA/S]) following each dose (when the PsE have subsided and 60 minutes after each study drug dosing) and as part of the discharge evaluation on Day 0;
   The incidence of adverse events (AEs) of mania or hypomania (as assessed using the DSM-5 criteria for mania/hypomania);
   Change from baseline in YMRS assessed as part of the discharge evaluation on Day 0 and at Day 1 and Day 7;
   Change from baseline in Clinician Administered Dissociative States Scale (CADSS) assessed as part of the discharge evaluation on Day 0 and at Day 1 and Day 7;
   Assessment of patient discharge readiness at discharge on Day 0 using the Clinical Assessment of Discharge Readiness (CADR);
   Change from baseline in Brief Psychiatric Rating Scale (BPRS) assessed as part of the discharge evaluation on Day 0, and at Day 1 and Day 7;
   C-SSRS categorization based on the Columbia Classification Algorithm of Suicide Assessment (C-CASA).
The PsE experienced by the patients as reported 30 to 60 minutes after each dosing, when the PsE has subsided:
   PsE assessment using the peak experience (PE) Scale (PES) to assess the achievement of a PE (PES total score·75);
   Challenging Experience Questionnaire (CEQ);
   Mystical Experience Questionnaire (MEQ-30).
Duration of the PsE, defined as the time from study drug dosing to the time when the PsE have subsided, completed 30 to 60 minutes after each dosing.
The impact on sleep quality as evaluated by change from pre-test day (Day-1) to Day 1 and to Day 7 in the Pittsburgh Sleep Quality Index (PSQI).
The impact on cognitive outcomes as evaluated by change from the pre-test day (Day-1) to discharge on Day 0, to Day 1 and to Day 7 in:
   Rapid visual information processing (RVP) test;
   Verbal recognition memory (VRM) test;
   Spatial working memory (SWM) test;
   Digit symbol substitution test (DSST).

1. 5-Methoxy-N,N-dimethyltryptamine (5-MeO-DMT) or a pharmaceutically acceptable salt thereof for use in treating a patient who is diagnosed with bipolar disorder, wherein the 5-MeO-DMT is administered via the intravenous, intramuscular or subcutaneous route.

2. 5-MeO-DMT or a pharmaceutically acceptable salt thereof for use as in claim 1, wherein the patient is diagnosed with bipolar II disorder.

3. 5-MeO-DMT or a pharmaceutically acceptable salt thereof for use as in claim 1, wherein the patient is diagnosed with bipolar I disorder.

4. 5-MeO-DMT or a pharmaceutically acceptable salt thereof for use as in claims 1 to 3, wherein the patient suffers from a current major depressive episode.

5. 5-MeO-DMT or a pharmaceutically acceptable salt thereof for use as in claim 4, wherein the patient has a Montgomery-Åsberg Depression Rating Scale (MADRS) total score of equal to or greater than 19, such as equal to or greater than 24, in particular equal to or greater than 37.

6. 5-MeO-DMT or a pharmaceutically acceptable salt thereof for use as in claim 4 or claim 5, wherein the patient has a Bipolar Depression Rating Scale (BDRS) total score of equal to or greater than 19, such as equal to or greater than 24, in particular equal to or greater than 37.

7. 5-MeO-DMT or a pharmaceutically acceptable salt thereof for use as in claims 1 to 6, wherein the patient had no adequate improvement after at least two adequate courses of therapy.

8. 5-MeO-DMT or a pharmaceutically acceptable salt thereof for use as in claims 1 to 6, wherein the patient had no adequate improvement after at least two adequate courses of therapy, wherein at least one of the two courses was a pharmacotherapy.

9. 5-MeO-DMT or a pharmaceutically acceptable salt thereof for use as in claims 1 to 6, wherein the patient had no adequate improvement after at least two adequate courses of pharmacotherapy.

10. 5-MeO-DMT or a pharmaceutically acceptable salt thereof for use as in claims 1 to 9, wherein the patient has a Young Mania Rating Scale (YMRS) total score less than or equal to 8.

11. 5-MeO-DMT or a pharmaceutically acceptable salt thereof for use as in claims 1 to 10, wherein the 5-MeO-DMT or salt thereof is administered at a dose or in a dosage regimen that causes the patient to experience a peak psychedelic experience.

12. 5-MeO-DMT or a pharmaceutically acceptable salt thereof for use as in claims 1 to 11, wherein a dosage of about 1 mg to about 10 mg 5-MeO-DMT is administered, or wherein equimolar amounts of the pharmaceutically acceptable salt are administered instead of 5-MeO-DMT.

13. 5-MeO-DMT or a pharmaceutically acceptable salt thereof for use as in claims 1 to 11, wherein a dosage of about 2 mg; or of about 5 mg; or of about 8 mg is administered, or wherein equimolar amounts of the pharmaceutically acceptable salt are administered instead of 5-MeO-DMT.

14. 5-MeO-DMT or a pharmaceutically acceptable salt thereof for use as in claims 1 to 11, wherein a dosage of about 1 mg; or of about 2 mg; or of about 3 mg is administered, or wherein equimolar amounts of the pharmaceutically acceptable salt are administered instead of 5-MeO-DMT.

15. 5-MeO-DMT or a pharmaceutically acceptable salt thereof for use as in claims 1 to 12, wherein the 5-MeO-DMT or salt thereof is administered in a first dosage amount for a first administration; and the 5-MeO-DMT or salt thereof is administered in zero to six subsequent administrations; wherein each subsequent administration uses a dosage amount higher than the previous administration unless the patient experiences a peak psychedelic experience.

16. 5-MeO-DMT or a pharmaceutically acceptable salt thereof for use as in claim 1 to 12 or 15, wherein the 5-MeO-DMT is administered in a dosage from about 1 mg to about 3 mg for a first administration, and then increased, unless the patient has already experienced a peak psychedelic experience, to a dosage from about 4 mg to about 6 mg for a second administration, and then increased, unless the patient has already experienced a peak psychedelic experience, to a dosage from about 7 mg to about 9 mg for a third administration, or wherein equimolar amounts of the pharmaceutically acceptable salt are administered instead of 5-MeO-DMT.

17. 5-MeO-DMT or a pharmaceutically acceptable salt thereof for use as in claim 16, wherein the first dosage of 5-MeO-DMT is about 2 mg, the second dosage of 5-MeO-DMT is about 5 mg, and the third dosage of 5-MeO-DMT is about 8 mg; or wherein equimolar amounts of the pharmaceutically acceptable salt are administered instead of 5-MeO-DMT.

18. 5-MeO-DMT or a pharmaceutically acceptable salt thereof for use as in claim 1 to 12 or 15, wherein the 5-MeO-DMT is administered in a dosage from about 0.5 mg to about 1.5 mg for a first administration, and then increased, unless the patient has already experienced a peak psychedelic experience, to a dosage from about 1.5 mg to about 2.5 mg for a second administration, and then increased, unless the patient has already experienced a peak psychedelic experience, to a dosage from about 2.5 mg to about 3.5 mg for a third administration, or wherein equimolar amounts of the pharmaceutically acceptable salt are administered instead of 5-MeO-DMT.

19. 5-MeO-DMT or a pharmaceutically acceptable salt thereof for use as in claim 18, wherein the first dosage of 5-MeO-DMT is about 1 mg, the second dosage of 5-MeO-DMT is about 2 mg, and the third dosage of 5-MeO-DMT is about 3 mg; or wherein equimolar amounts of the pharmaceutically acceptable salt are administered instead of 5-MeO-DMT.

20. 5-MeO-DMT or a pharmaceutically acceptable salt thereof for use as in claims 15 to 19, wherein the interval between two administrations is not less than 1 hour and not more than 24 hours, such as about 1 to 4 hours, preferably 1 to 2 hours.

21. 5-MeO-DMT or a pharmaceutically acceptable salt thereof for use as in claims 11 to 20, wherein the occurrence of a peak psychedelic experience is identified through achievement of at least 60% of the maximum possible score in each of the four subscales (mystical, positive mood, transcendence of time and space, and ineffability) of the 30-item revised Mystical Experience Questionnaire (MEQ30) or is identified through achievement of at least 60% of the maximum possible score of the Oceanic Boundlessness (OBN) dimension of the Altered States of Consciousness (ASC) questionnaire or is identified through achievement of a Peak Experience Scale (PES) Total Score of at least 75.

22. 5-MeO-DMT or a pharmaceutically acceptable salt thereof for use as in claim 21, wherein the occurrence of a peak psychedelic experience is identified through achievement of a Peak Experience Scale (PES) Total Score of at least 75.

23. 5-MeO-DMT or a pharmaceutically acceptable salt thereof for use as in claims 1 to 22, wherein the 5-MeO-DMT or a pharmaceutically acceptable salt thereof is administered via intravenous injection.

24. 5-MeO-DMT or a pharmaceutically acceptable salt thereof for use as in claims 1 to 23, wherein a clinical response, as reflected by a reduction in the Clinical Global Impression-Severity (CGI-S) score, occurs not later than about 2 hours after the last administration of 5-MeO-DMT or a pharmaceutically acceptable salt thereof.

25. 5-MeO-DMT or a pharmaceutically acceptable salt thereof for use as in claims 1 to 24, wherein a clinical response, as reflected by a reduction in the Clinical Global Impression-Severity (CGI-S) score, is observed on day 1, for instance, about 24 hours after the last administration of 5-MeO-DMT or a pharmaceutically acceptable salt thereof.

26. 5-MeO-DMT or a pharmaceutically acceptable salt thereof for use as in claim 1 or 25, wherein a clinical response, as reflected by a reduction in the Clinical Global Impression-Severity (CGI-S) score, persists until at least 6 days after the last administration of 5-MeO-DMT or a pharmaceutically acceptable salt thereof.

27. 5-MeO-DMT or a pharmaceutically acceptable salt thereof for use as in claims 1 to 26, wherein a clinical response, as reflected by a reduction in the Clinical Global Impression-Severity (CGI-S) score, persists until at least 14 days after the last administration of 5-MeO-DMT or a pharmaceutically acceptable salt thereof.

28. 5-MeO-DMT or a pharmaceutically acceptable salt thereof for use as in claims 1 to 27, wherein a clinical response, as reflected by a reduction in the Clinical Global Impression-Severity (CGI-S) score, persists until at least 28 days after the last administration of 5-MeO-DMT or a pharmaceutically acceptable salt thereof.

29. 5-MeO-DMT or a pharmaceutically acceptable salt thereof for use as in claims 1 to 28, wherein a clinical response, as reflected by at least 50% improvement of the BDRS; the MADRS or the HAM-D score, compared to the respective score prior to treatment, occurs not later than about 2 hours after the last administration of 5-MeO-DMT or a pharmaceutically acceptable salt thereof.

30. 5-MeO-DMT or a pharmaceutically acceptable salt thereof for use as in claims 1 to 29, wherein a remission of depressive symptoms, as reflected by a BDRS score equal to or less than 10; a MADRS score equal to or less than 10, or a HAM-D score equal to or less than 7, occurs not later than about 2 hours after the last administration of 5-MeO-DMT or a pharmaceutically acceptable salt thereof.

31. 5-MeO-DMT or a pharmaceutically acceptable salt thereof for use as in claims 1 to 30, wherein a clinical response, as reflected by at least 50% improvement of the BDRS; the MADRS or the HAM-D score, compared to the respective score prior to treatment, is observed on day 1, for instance, about 24 hours after the last administration of 5-MeO-DMT or a pharmaceutically acceptable salt thereof.

32. 5-MeO-DMT or a pharmaceutically acceptable salt thereof for use as in claims 1 to 31, wherein a remission of depressive symptoms, as reflected by a BDRS score equal to or less than 10; a MADRS score equal to or less than 10, or a HAM-D score equal to or less than 7, is observed on day 1, for instance, about 24 hours after the last administration of 5-MeO-DMT or a pharmaceutically acceptable salt thereof.

33. 5-MeO-DMT or a pharmaceutically acceptable salt thereof for use as in claims 1 to 32, wherein a clinical response, as reflected by at least 50% improvement of the BDRS; the MADRS or the HAM-D score, compared to the respective score prior to treatment, persists until at least 6 days after the last administration of 5-MeO-DMT or a pharmaceutically acceptable salt thereof.

34. 5-MeO-DMT or a pharmaceutically acceptable salt thereof for use as in claims 1 to 33, wherein there is a clinical response, as reflected by at least 75% improvement of the BDRS; the MADRS or the HAM-D score, compared to the respective score prior to treatment, on day 7 after the last administration of 5-MeO-DMT or a pharmaceutically acceptable salt thereof.

35. 5-MeO-DMT or a pharmaceutically acceptable salt thereof for use as in claims 1 to 34, wherein the patient is in remission of depressive symptoms, as reflected by a BDRS score equal to or less than 10; a MADRS score equal to or less than 10, or a HAM-D score equal to or less than 7, on day 7 after the last administration of 5-MeO-DMT or a pharmaceutically acceptable salt thereof.

36. 5-MeO-DMT or a pharmaceutically acceptable salt thereof for use as in claims 1 to 35, wherein a clinical response, as reflected by at least 50% improvement of the BDRS; the MADRS or the HAM-D score, compared to the respective score prior to treatment, persists until at least 14 days after the last administration of 5-MeO-DMT or a pharmaceutically acceptable salt thereof.

37. 5-MeO-DMT or a pharmaceutically acceptable salt thereof for use as in claims 1 to 36, wherein there is a clinical response, as reflected by at least 75% improvement of the BDRS; the MADRS or the HAM-D score, compared to the respective score prior to treatment, on day 14 after the last administration of 5-MeO-DMT or a pharmaceutically acceptable salt thereof.

38. 5-MeO-DMT or a pharmaceutically acceptable salt thereof for use as in claims 1 to 37, wherein the patient is in remission of depressive symptoms, as reflected by a BDRS score equal to or less than 10; a MADRS score equal to or less than 10, or a HAM-D score equal to or less than 7, on day 14 after the last administration of 5-MeO-DMT or a pharmaceutically acceptable salt there.

39. 5-MeO-DMT or a pharmaceutically acceptable salt thereof for use as in claims 1 to 38, wherein a clinical response, as reflected by at least 50% improvement of the BDRS; the MADRS or the HAM-D score, compared to the respective score prior to treatment, persists until at least 28 days after the last administration of 5-MeO-DMT or a pharmaceutically acceptable salt thereof.

40. 5-MeO-DMT or a pharmaceutically acceptable salt thereof for use as in claims 1 to 39, wherein there is a clinical response, as reflected by at least 75% improvement of the BDRS; the MADRS or the HAM-D score, compared to the respective score prior to treatment, on day 28 after the last administration of 5-MeO-DMT or a pharmaceutically acceptable salt thereof.

41. 5-MeO-DMT or a pharmaceutically acceptable salt thereof for use as in claims 1 to 40, wherein the patient is in remission of depressive symptoms, as reflected by a BDRS score equal to or less than 10; a MADRS score equal to or less than 10, or a HAM-D score equal to or less than 7, on day 28 after the last administration of 5-MeO-DMT or a pharmaceutically acceptable salt thereof.

42. 5-MeO-DMT or a pharmaceutically acceptable salt thereof for use as in claims 1 to 41, wherein the patient does not experience treatment-emergent mania or hypomania.

43. 5-MeO-DMT or a pharmaceutically acceptable salt thereof for use as in claims 1 to 42, wherein the patient has a Young Mania Rating Scale (YMRS) total score less than or equal to 15, preferably less than or equal to 12, as assessed about 2 hours after the last administration of 5-MeO-DMT or a pharmaceutically acceptable salt thereof.

44. 5-MeO-DMT or a pharmaceutically acceptable salt thereof for use as in claims 1 to 43, wherein the patient has a Young Mania Rating Scale (YMRS) total score less than or equal to 15, preferably less than or equal to 12, as assessed on day 1, for instance, about 24 hours after the last administration of 5-MeO-DMT or a pharmaceutically acceptable salt thereof.

45. 5-MeO-DMT or a pharmaceutically acceptable salt thereof for use as in claims 1 to 44, wherein the patient has a Young Mania Rating Scale (YMRS) total score less than or equal to 15, preferably less than or equal to 12, as assessed on day 7 after the last administration of 5-MeO-DMT or a pharmaceutically acceptable salt thereof.

46. 5-MeO-DMT or a pharmaceutically acceptable salt thereof for use as in claims 1 to 45, wherein the patient has a Young Mania Rating Scale (YMRS) total score less than or equal to 15, preferably less than or equal to 12, as assessed on day 14 after the last administration of 5-MeO-DMT or a pharmaceutically acceptable salt thereof.

47. 5-MeO-DMT or a pharmaceutically acceptable salt thereof for use as in claims 1 to 46, wherein the patient has a Young Mania Rating Scale (YMRS) total score less than or equal to 15, preferably less than or equal to 12, as assessed on day 28 after the last administration of 5-MeO-DMT or a pharmaceutically acceptable salt thereof.

48. 5-MeO-DMT or a pharmaceutically acceptable salt thereof for use as in claims 1 to 47, wherein the treatment leads to an improvement in at least one of sleep disturbance, psychomotor retardation, negative thinking, anxiety, cognitive dysfunction, and social/emotional withdrawal or detachment.

49. 5-MeO-DMT or a pharmaceutically acceptable salt thereof for use as in claims 1 to 48, wherein the patient suffers from sleep disturbance and the treatment reduces or eliminates the sleep disturbance.

50. 5-MeO-DMT or a pharmaceutically acceptable salt thereof for use as in claim 49, wherein the patient suffers from insomnia and the treatment reduces or eliminates insomnia.

51. 5-MeO-DMT or a pharmaceutically acceptable salt thereof for use as in claim 49, wherein the patient suffers from hypersomnia and the treatment reduces or eliminates hypersomnia.

52. 5-MeO-DMT or a pharmaceutically acceptable salt thereof for use as in claims 49 to 51, wherein the reduction or elimination of sleep disturbance is reflected by an improvement at least in the score of the BDRS item sleep disturbance on day 1, for instance, about 24 hours after the last administration of 5-MeO-DMT or a pharmaceutically acceptable salt thereof.

53. 5-MeO-DMT or a pharmaceutically acceptable salt thereof for use as in claims 49 to 52, wherein the reduction or elimination of sleep disturbance is reflected by an improvement at least in the score of the BDRS item sleep disturbance on day 7 after the last administration of 5-MeO-DMT or a pharmaceutically acceptable salt thereof.

54. 5-MeO-DMT or a pharmaceutically acceptable salt thereof for use as in claims 49 to 53, wherein the reduction or elimination of sleep disturbance is reflected by an improvement at least in the score of the BDRS item sleep disturbance on day 14 after the last administration of 5-MeO-DMT or a pharmaceutically acceptable salt thereof.

55. 5-MeO-DMT or a pharmaceutically acceptable salt thereof for use as in claims 49 to 54, wherein the reduction or elimination of sleep disturbance is reflected by an improvement at least in the score of the BDRS item sleep disturbance on day 28 after the last administration of 5-MeO-DMT or a pharmaceutically acceptable salt thereof.

56. 5-MeO-DMT or a pharmaceutically acceptable salt thereof for use as in claim 49, wherein the reduction or elimination of sleep disturbance as reflected by an improvement in the score of the BDRS item sleep disturbance occurs not later than about 24 hours after the last administration of 5-MeO-DMT or a pharmaceutically acceptable salt thereof.

57. 5-MeO-DMT or a pharmaceutically acceptable salt thereof for use as in claim 56, wherein the reduction or elimination of sleep disturbance as reflected by an improvement in the score of the BDRS item sleep disturbance persists until at least 6 days after the last administration of 5-MeO-DMT or a pharmaceutically acceptable salt thereof.

58. 5-MeO-DMT or a pharmaceutically acceptable salt thereof for use as in claim 56, wherein the reduction or elimination of sleep disturbance as reflected by an improvement in the score of the BDRS item sleep disturbance persists until at least 14 days after the last administration of 5-MeO-DMT or a pharmaceutically acceptable salt thereof.

59. 5-MeO-DMT or a pharmaceutically acceptable salt thereof for use as in claim 56, wherein the reduction or elimination of sleep disturbance as reflected by an improvement in the score of the BDRS item sleep disturbance persists until at least 28 days after the last administration of 5-MeO-DMT or a pharmaceutically acceptable salt thereof.

60. 5-MeO-DMT or a pharmaceutically acceptable salt thereof for use as in claim 49, wherein the sleep disturbance is reduced sleep and the reduction or elimination of sleep disturbance is reflected by an improvement at least in the score of the MADRS item reduced sleep on day 1, for instance, about 24 hours after the last administration of 5-MeO-DMT or a pharmaceutically acceptable salt thereof.

61. 5-MeO-DMT or a pharmaceutically acceptable salt thereof for use as in claim 49 or 60, wherein the sleep disturbance is reduced sleep and the reduction or elimination of sleep disturbance is reflected by an improvement at least in the score of the MADRS item reduced sleep on day 7 after the last administration of 5-MeO-DMT or a pharmaceutically acceptable salt thereof.

62. 5-MeO-DMT or a pharmaceutically acceptable salt thereof for use as in claim 49, 60 or 61, wherein the sleep disturbance is reduced sleep and the reduction or elimination of sleep disturbance is reflected by an improvement at least in the score of the MADRS item reduced sleep on day 14 after the last administration of 5-MeO-DMT or a pharmaceutically acceptable salt thereof.

63. 5-MeO-DMT or a pharmaceutically acceptable salt thereof for use as in claim 49 or 60 to 62, wherein the sleep disturbance is reduced sleep and the reduction or elimination of sleep disturbance is reflected by an improvement at least in the score of the MADRS item reduced sleep on day 28 after the last administration of 5-MeO-DMT or a pharmaceutically acceptable salt thereof.

64. 5-MeO-DMT or a pharmaceutically acceptable salt thereof for use as in claim 49, wherein the sleep disturbance is reduced sleep and the reduction or elimination of sleep disturbance as reflected by an improvement in the score of the MADRS item reduced sleep occurs not later than about 24 hours after the last administration of 5-MeO-DMT or a pharmaceutically acceptable salt thereof.

65. 5-MeO-DMT or a pharmaceutically acceptable salt thereof for use as in claim 64, wherein the sleep disturbance is reduced sleep and the reduction or elimination of sleep disturbance as reflected by an improvement in the score of the MADRS item reduced sleep persists until at least 6 days after the last administration of 5-MeO-DMT or a pharmaceutically acceptable salt thereof.

66. 5-MeO-DMT or a pharmaceutically acceptable salt thereof for use as in claim 64, wherein the sleep disturbance is reduced sleep and the reduction or elimination of sleep disturbance as reflected by an improvement in the score of the MADRS item reduced sleep persists until at least 14 days after the last administration of 5-MeO-DMT or a pharmaceutically acceptable salt thereof.

67. 5-MeO-DMT or a pharmaceutically acceptable salt thereof for use as in claim 64, wherein the sleep disturbance is reduced sleep and the reduction or elimination of sleep disturbance as reflected by an improvement in the score of the MADRS item reduced sleep persists until at least 28 days after the last administration of 5-MeO-DMT or a pharmaceutically acceptable salt thereof.

68. 5-MeO-DMT or a pharmaceutically acceptable salt thereof for use as in claim 49, wherein the patient suffers from sleep disturbance and an improvement in sleep disturbance, is reflected by a reduction in the Clinical Global Impression-Severity (CGI-S) score on day 1, for instance, about 24 hours after the last administration of 5-MeO-DMT or a pharmaceutically acceptable salt thereof.

69. 5-MeO-DMT or a pharmaceutically acceptable salt thereof for use as in claim 49 or 68, wherein the patient suffers from sleep disturbance and an improvement in sleep disturbance is reflected by a reduction in the Clinical Global Impression-Severity (CGI-S) score on day 7 after the last administration of 5-MeO-DMT or a pharmaceutically acceptable salt thereof.

70. 5-MeO-DMT or a pharmaceutically acceptable salt thereof for use as in claim 49, 68 or 69, wherein the patient suffers from sleep disturbance and an improvement in sleep disturbance is reflected by a reduction in the Clinical Global Impression-Severity (CGI-S) score on day 14 after the last administration of 5-MeO-DMT or a pharmaceutically acceptable salt thereof.

71. 5-MeO-DMT or a pharmaceutically acceptable salt thereof for use as in claim 49 or 68 to 70, wherein the patient suffers from sleep disturbance and an improvement in sleep disturbance is reflected by a reduction in the Clinical Global Impression-Severity (CGI-S) score on day 28 after the last administration of 5-MeO-DMT or a pharmaceutically acceptable salt thereof.

72. 5-MeO-DMT or a pharmaceutically acceptable salt thereof for use as in claims 1 to 48, wherein the patient suffers from sleep disturbance and an improvement in sleep disturbance, as reflected by a reduction in the Clinical Global Impression-Severity (CGI-S) score, occurs not later than about 24 hours after the last administration of 5-MeO-DMT or a pharmaceutically acceptable salt thereof.

73. 5-MeO-DMT or a pharmaceutically acceptable salt thereof for use as in claim 72, wherein the improvement in sleep disturbance, as reflected by a reduction in the Clinical Global Impression-Severity (CGI-S) score, persists until at least 6 days after the last administration of 5-MeO-DMT or a pharmaceutically acceptable salt thereof.

74. 5-MeO-DMT or a pharmaceutically acceptable salt thereof for use as in claim 72, wherein the improvement in sleep disturbance, as reflected by a reduction in the Clinical Global Impression-Severity (CGI-S) score, persists until at least 14 days after the last administration of 5-MeO-DMT or a pharmaceutically acceptable salt thereof.

75. 5-MeO-DMT or a pharmaceutically acceptable salt thereof for use as in claim 72, wherein the improvement in sleep disturbance, as reflected by a reduction in the Clinical Global Impression-Severity (CGI-S) score, persists until at least 28 days after the last administration of 5-MeO-DMT or a pharmaceutically acceptable salt thereof.

76. 5-MeO-DMT or a pharmaceutically acceptable salt thereof for use as in claims 1 to 48, wherein the patient suffers from sleep disturbance and the reduction or elimination of sleep disturbance is reflected by an improvement in the Pittsburgh Sleep Quality Index (PSQI) global score on day 1, for instance, about 24 hours after the last administration of 5-MeO-DMT or a pharmaceutically acceptable salt thereof, wherein the recall period spans from the time point when acute psychedelic experiences have subsided after the last administration to the assessment time point.

77. 5-MeO-DMT or a pharmaceutically acceptable salt thereof for use as in claim 1 to 48 or 76, wherein the patient suffers from sleep disturbance and the reduction or elimination of sleep disturbance is reflected by an improvement in the Pittsburgh Sleep Quality Index (PSQI) global score on day 7 after the last administration of 5-MeO-DMT or a pharmaceutically acceptable salt thereof, wherein the recall period spans from the time point when acute psychedelic experiences have subsided after the last administration to the assessment time point.

78. 5-MeO-DMT or a pharmaceutically acceptable salt thereof for use as in claim 1 to 48, 76 or 77, wherein the patient suffers from sleep disturbance and the reduction or elimination of sleep disturbance is reflected by an improvement in the Pittsburgh Sleep Quality Index (PSQI) global score on day 14 after the last administration of 5-MeO-DMT or a pharmaceutically acceptable salt thereof, wherein the recall period spans from the time point when acute psychedelic experiences have subsided after the last administration to the assessment time point.

79. 5-MeO-DMT or a pharmaceutically acceptable salt thereof for use as in claims 1 to 48 or 76 to 78, wherein the patient suffers from sleep disturbance and the reduction or elimination of sleep disturbance is reflected by an improvement in the Pittsburgh Sleep Quality Index (PSQI) global score on day 28 after the last administration of 5-MeO-DMT or a pharmaceutically acceptable salt thereof, wherein the recall period spans from the time point when acute psychedelic experiences have subsided after the last administration to the assessment time point.

80. 5-MeO-DMT or a pharmaceutically acceptable salt thereof for use as in claim 49, wherein the patient suffers from sleep disturbance and the reduction or elimination of sleep disturbance as reflected by an improvement in the Pittsburgh Sleep Quality Index (PSQI) global score occurs not later than about 24 hours after the last administration of 5-MeO-DMT or a pharmaceutically acceptable salt thereof, wherein the recall period spans from the time point when acute psychedelic experiences have subsided after the last administration to the assessment time point.

81. 5-MeO-DMT or a pharmaceutically acceptable salt thereof for use as in claim 80, wherein the patient suffers from sleep disturbance and the reduction or elimination of sleep disturbance as reflected by an improvement in the Pittsburgh Sleep Quality Index (PSQI) global score persists until at least 6 days after the last administration of 5-MeO-DMT or a pharmaceutically acceptable salt thereof, wherein the recall period spans from the time point when acute psychedelic experiences have subsided after the last administration to the assessment time point.

82. 5-MeO-DMT or a pharmaceutically acceptable salt thereof for use as in claim 80, wherein the patient suffers from sleep disturbance and the reduction or elimination of sleep disturbance as reflected by an improvement in the Pittsburgh Sleep Quality Index (PSQI) global score persists until at least 14 days after the last administration of 5-MeO-DMT or a pharmaceutically acceptable salt thereof, wherein the recall period spans from the time point when acute psychedelic experiences have subsided after the last administration to the assessment time point.

83. 5-MeO-DMT or a pharmaceutically acceptable salt thereof for use as in claim 80, wherein the patient suffers from sleep disturbance and the reduction or elimination of sleep disturbance as reflected by an improvement in the Pittsburgh Sleep Quality Index (PSQI) global score persists until at least 28 days after the last administration of 5-MeO-DMT or a pharmaceutically acceptable salt thereof, wherein the recall period spans from the time point when acute psychedelic experiences have subsided after the last administration to the assessment time point.

84. 5-MeO-DMT or a pharmaceutically acceptable salt thereof for use as in claims 1 to 83, wherein the patient suffers from psychomotor retardation and the treatment reduces or eliminates the psychomotor retardation.

85. 5-MeO-DMT or a pharmaceutically acceptable salt thereof for use as in claim 84, wherein the treatment improves or eliminates reduced energy and activity and/or reduced motivation.

86. 5-MeO-DMT or a pharmaceutically acceptable salt thereof for use as in claim 84 or 85, wherein the reduction or elimination of psychomotor retardation is reflected by an improvement at least in the score of the BDRS items reduced energy and activity and/or reduced motivation about 2 hours after the last administration of 5-MeO-DMT or a pharmaceutically acceptable salt thereof.

87. 5-MeO-DMT or a pharmaceutically acceptable salt thereof for use as in claims 84 to 86, wherein the reduction or elimination of psychomotor retardation is reflected by an improvement at least in the score of the BDRS items reduced energy and activity and/or reduced motivation on day 1, for instance, about 24 hours after the last administration of 5-MeO-DMT or a pharmaceutically acceptable salt thereof.

88. 5-MeO-DMT or a pharmaceutically acceptable salt thereof for use as in claims 84 to 87, wherein the reduction or elimination of psychomotor retardation is reflected by an improvement at least in the score of the BDRS items reduced energy and activity and/or reduced motivation on day 7 after the last administration of 5-MeO-DMT or a pharmaceutically acceptable salt thereof.

89. 5-MeO-DMT or a pharmaceutically acceptable salt thereof for use as in claims 84 to 88, wherein the reduction or elimination of psychomotor retardation is reflected by an improvement at least in the score of the BDRS items reduced energy and activity and/or reduced motivation on day 14 after the last administration of 5-MeO-DMT or a pharmaceutically acceptable salt thereof.

90. 5-MeO-DMT or a pharmaceutically acceptable salt thereof for use as in claims 84 to 89, wherein the reduction or elimination of psychomotor retardation is reflected by an improvement at least in the score of the BDRS items reduced energy and activity and/or reduced motivation on day 28 after the last administration of 5-MeO-DMT or a pharmaceutically acceptable salt thereof.

91. 5-MeO-DMT or a pharmaceutically acceptable salt thereof for use as in claim 84 or 85, wherein the reduction or elimination of psychomotor retardation as reflected by an improvement in the score of the BDRS items reduced energy and activity and/or reduced motivation occurs not later than about 2 hours after the last administration of 5-MeO-DMT or a pharmaceutically acceptable salt thereof.

92. 5-MeO-DMT or a pharmaceutically acceptable salt thereof for use as in claim 91, wherein the reduction or elimination of psychomotor retardation as reflected by an improvement in the score of the BDRS items reduced energy and activity and/or reduced motivation persists until at least 6 days after the last administration of 5-MeO-DMT or a pharmaceutically acceptable salt thereof.

93. 5-MeO-DMT or a pharmaceutically acceptable salt thereof for use as in claim 91, wherein the reduction or elimination of psychomotor retardation as reflected by an improvement in the score of the BDRS items reduced energy and activity and/or reduced motivation persists until at least 14 days after the last administration of 5-MeO-DMT or a pharmaceutically acceptable salt thereof.

94. 5-MeO-DMT or a pharmaceutically acceptable salt thereof for use as in claim 91, wherein the reduction or elimination of psychomotor retardation as reflected by an improvement in the score of the BDRS items reduced energy and activity and/or reduced motivation persists until at least 28 days after the last administration of 5-MeO-DMT or a pharmaceutically acceptable salt there.

95. 5-MeO-DMT or a pharmaceutically acceptable salt thereof for use as in claim 84, wherein the reduction or elimination of psychomotor retardation is reflected by an improvement at least in the score of the MADRS item lassitude about 2 hours after the last administration of 5-MeO-DMT or a pharmaceutically acceptable salt thereof.

96. 5-MeO-DMT or a pharmaceutically acceptable salt thereof for use as in claim 84 or 95, wherein the reduction or elimination of psychomotor retardation is reflected by an improvement at least in the score of the MADRS item lassitude on day 1, for instance, about 24 hours after the last administration of 5-MeO-DMT or a pharmaceutically acceptable salt thereof.

97. 5-MeO-DMT or a pharmaceutically acceptable salt thereof for use as in claim 84, 95 or 96, wherein the reduction or elimination of psychomotor retardation is reflected by an improvement at least in the score of the MADRS item lassitude on day 7 after the last administration of 5-MeO-DMT or a pharmaceutically acceptable salt thereof.

98. 5-MeO-DMT or a pharmaceutically acceptable salt thereof for use as in claim 84 or 95 to 97, wherein the reduction or elimination of psychomotor retardation is reflected by an improvement at least in the score of the MADRS item lassitude on day 14 after the last administration of 5-MeO-DMT or a pharmaceutically acceptable salt thereof.

99. 5-MeO-DMT or a pharmaceutically acceptable salt thereof for use as in claim 84 or 95 to 98, wherein the reduction or elimination of psychomotor retardation is reflected by an improvement at least in the score of the MADRS item lassitude on day 28 after the last administration of 5-MeO-DMT or a pharmaceutically acceptable salt thereof.

100. 5-MeO-DMT or a pharmaceutically acceptable salt thereof for use as in claim 84, wherein the reduction or elimination of psychomotor retardation as reflected by an improvement in the score of the MADRS item lassitude occurs not later than about 2 hours after the last administration of 5-MeO-DMT or a pharmaceutically acceptable salt thereof.

101. 5-MeO-DMT or a pharmaceutically acceptable salt thereof for use as in claim 100, wherein the reduction or elimination of psychomotor retardation as reflected by an improvement in the score of the MADRS item lassitude persists until at least 6 days after the last administration of 5-MeO-DMT or a pharmaceutically acceptable salt thereof.

102. 5-MeO-DMT or a pharmaceutically acceptable salt thereof for use as in claim 100, wherein the reduction or elimination of psychomotor retardation as reflected by an improvement in the score of the MADRS item lassitude persists until at least 14 days after the last administration of 5-MeO-DMT or a pharmaceutically acceptable salt thereof.

103. 5-MeO-DMT or a pharmaceutically acceptable salt thereof for use as in claim 100, wherein the reduction or elimination of psychomotor retardation as reflected by an improvement in the score of the MADRS item lassitude persists until at least 28 days after the last administration of 5-MeO-DMT or a pharmaceutically acceptable salt thereof.

104. 5-MeO-DMT or a pharmaceutically acceptable salt thereof for use as in claim 84, wherein the patient suffers from psychomotor retardation and an improvement in psychomotor retardation is reflected by a reduction in the Clinical Global Impression-Severity (CGI-S) score about 2 hours after the last administration of 5-MeO-DMT or a pharmaceutically acceptable salt thereof.

105. 5-MeO-DMT or a pharmaceutically acceptable salt thereof for use as in claim 84 or 104, wherein the patient suffers from psychomotor retardation and an improvement in psychomotor retardation is reflected by a reduction in the Clinical Global Impression-Severity (CGI-S) score on day 1, for instance, about 24 hours after the last administration of 5-MeO-DMT or a pharmaceutically acceptable salt thereof.

106. 5-MeO-DMT or a pharmaceutically acceptable salt thereof for use as in claim 84, 104 or 105, wherein the patient suffers from psychomotor retardation and an improvement in psychomotor retardation is reflected by a reduction in the Clinical Global Impression-Severity (CGI-S) score on day 7 after the last administration of 5-MeO-DMT or a pharmaceutically acceptable salt thereof.

107. 5-MeO-DMT or a pharmaceutically acceptable salt thereof for use as in claim 84 or 104 to 106, wherein the patient suffers from psychomotor retardation and an improvement in psychomotor retardation is reflected by a reduction in the Clinical Global Impression-Severity (CGI-S) score on day 14 after the last administration of 5-MeO-DMT or a pharmaceutically acceptable salt thereof.

108. 5-MeO-DMT or a pharmaceutically acceptable salt thereof for use as in claim 84 or 104 to 107, wherein the patient suffers from psychomotor retardation and an improvement in psychomotor retardation is reflected by a reduction in the Clinical Global Impression-Severity (CGI-S) score on day 28 after the last administration of 5-MeO-DMT or a pharmaceutically acceptable salt thereof.

109. 5-MeO-DMT or a pharmaceutically acceptable salt thereof for use as in claims 1 to 83, wherein the patient suffers from psychomotor retardation and an improvement in psychomotor retardation, as reflected by a reduction in the Clinical Global Impression-Severity (CGI-S) score, occurs not later than about 2 hours after the last administration of 5-MeO-DMT or a pharmaceutically acceptable salt thereof.

110. 5-MeO-DMT or a pharmaceutically acceptable salt thereof for use as in claim 109, wherein the improvement in psychomotor retardation, as reflected by a reduction in the Clinical Global Impression-Severity (CGI-S) score, persists until at least 6 days after the last administration of 5-MeO-DMT or a pharmaceutically acceptable salt thereof.

111. 5-MeO-DMT or a pharmaceutically acceptable salt thereof for use as in claim 109, wherein the improvement in psychomotor retardation, as reflected by a reduction in the Clinical Global Impression-Severity (CGI-S) score, persists until at least 14 days after the last administration of 5-MeO-DMT or a pharmaceutically acceptable salt thereof.

112. 5-MeO-DMT or a pharmaceutically acceptable salt thereof for use as in claim 109, wherein the improvement in psychomotor retardation, as reflected by a reduction in the Clinical Global Impression-Severity (CGI-S) score, persists until at least 28 days after the last administration of 5-MeO-DMT or a pharmaceutically acceptable salt thereof.

113. 5-MeO-DMT or a pharmaceutically acceptable salt thereof for use as in claims 1 to 112, wherein the patient suffers from negative thinking and the treatment reduces or eliminates the negative thinking.

114. 5-MeO-DMT or a pharmaceutically acceptable salt thereof for use as in claim 113, wherein the treatment reduces or eliminates feelings of worthlessness; helplessness and hopelessness; and/or guilt.

115. 5-MeO-DMT or a pharmaceutically acceptable salt thereof for use as in claim 113 or 114, wherein the reduction or elimination of negative thinking is reflected by an improvement at least in the scores of the BDRS items worthlessness; helplessness and hopelessness; and/or guilt about 2 hours after the last administration of 5-MeO-DMT or a pharmaceutically acceptable salt thereof.

116. 5-MeO-DMT or a pharmaceutically acceptable salt thereof for use as in claims 113 to 115, wherein the reduction or elimination of negative thinking is reflected by an improvement at least in the scores of the BDRS items worthlessness; helplessness and hopelessness; and/or guilt on day 1, for instance, about 24 hours after the last administration of 5-MeO-DMT or a pharmaceutically acceptable salt thereof.

117. 5-MeO-DMT or a pharmaceutically acceptable salt thereof for use as in claims 113 to 116, wherein the reduction or elimination of negative thinking is reflected by an improvement at least in the scores of the BDRS items worthlessness; helplessness and hopelessness; and/or guilt on day 7 after the last administration of 5-MeO-DMT or a pharmaceutically acceptable salt thereof.

118. 5-MeO-DMT or a pharmaceutically acceptable salt thereof for use as in claims 113 to 117, wherein the reduction or elimination of negative thinking is reflected by an improvement at least in the scores of the BDRS items worthlessness; helplessness and hopelessness; and/or guilt on day 14 after the last administration of 5-MeO-DMT or a pharmaceutically acceptable salt thereof.

119. 5-MeO-DMT or a pharmaceutically acceptable salt thereof for use as in claims 113 to 118, wherein the reduction or elimination of negative thinking is reflected by an improvement at least in the score of the BDRS items worthlessness; helplessness and hopelessness; and/or guilt on day 28 after the last administration of 5-MeO-DMT or a pharmaceutically acceptable salt thereof.

120. 5-MeO-DMT or a pharmaceutically acceptable salt thereof for use as in claim 113 or 114, wherein the reduction or elimination of negative thinking as reflected by an improvement in the scores of the BDRS items worthlessness; helplessness and hopelessness; and/or guilt occurs not later than about 2 hours after the last administration of 5-MeO-DMT or a pharmaceutically acceptable salt thereof.

121. 5-MeO-DMT or a pharmaceutically acceptable salt thereof for use as in claim 120, wherein the reduction or elimination of negative thinking as reflected by an improvement in the scores of the BDRS items worthlessness; helplessness and hopelessness; and/or guilt persists until at least 6 days after the last administration of 5-MeO-DMT or a pharmaceutically acceptable salt thereof.

122. 5-MeO-DMT or a pharmaceutically acceptable salt thereof for use as in claim 120, wherein the reduction or elimination of negative thinking as reflected by an improvement in the scores of the BDRS items worthlessness; helplessness and hopelessness; and/or guilt persists until at least 14 days after the last administration of 5-MeO-DMT or a pharmaceutically acceptable salt thereof.

123. 5-MeO-DMT or a pharmaceutically acceptable salt thereof for use as in claim 120, wherein the reduction or elimination of negative thinking as reflected by an improvement in the scores of the BDRS items worthlessness; helplessness and hopelessness; and/or guilt persists until at least 28 days after the last administration of 5-MeO-DMT or a pharmaceutically acceptable salt thereof.

124. 5-MeO-DMT or a pharmaceutically acceptable salt thereof for use as in claim 113 or 114, wherein the reduction or elimination of negative thinking is reflected by an improvement at least in the score of the MADRS item pessimistic thoughts about 2 hours after the last administration of 5-MeO-DMT or a pharmaceutically acceptable salt thereof.

125. 5-MeO-DMT or a pharmaceutically acceptable salt thereof for use as in claim 113, 114 or 124, wherein the reduction or elimination of negative thinking is reflected by an improvement at least in the score of the MADRS item pessimistic thoughts on day 1, for instance, about 24 hours after the last administration of 5-MeO-DMT or a pharmaceutically acceptable salt thereof.

126. 5-MeO-DMT or a pharmaceutically acceptable salt thereof for use as in claim 113, 114, 124 or 125, wherein the reduction or elimination of negative thinking is reflected by an improvement at least in the score of the MADRS item pessimistic thoughts on day 7 after the last administration of 5-MeO-DMT or a pharmaceutically acceptable salt thereof.

127. 5-MeO-DMT or a pharmaceutically acceptable salt thereof for use as in claims 113, 114 or 124 to 126, wherein the reduction or elimination of negative thinking is reflected by an improvement at least in the score of the MADRS item pessimistic thoughts on day 14 after the last administration of 5-MeO-DMT or a pharmaceutically acceptable salt thereof.

128. 5-MeO-DMT or a pharmaceutically acceptable salt thereof for use as in claims 113, 114 or 124 to 127, wherein the reduction or elimination of negative thinking is reflected by an improvement at least in the score of the MADRS item pessimistic thoughts on day 28 after the last administration of 5-MeO-DMT or a pharmaceutically acceptable salt thereof.

129. 5-MeO-DMT or a pharmaceutically acceptable salt thereof for use as in claim 113 or 114, wherein the reduction or elimination of negative thinking as reflected by an improvement in the score of the MADRS item pessimistic thoughts occurs not later than about 2 hours after the last administration of 5-MeO-DMT or a pharmaceutically acceptable salt thereof.

130. 5-MeO-DMT or a pharmaceutically acceptable salt thereof for use as in claim 129, wherein the reduction or elimination of negative thinking as reflected by an improvement in the score of the MADRS item pessimistic thoughts persists until at least 6 days after the last administration of 5-MeO-DMT or a pharmaceutically acceptable salt thereof.

131. 5-MeO-DMT or a pharmaceutically acceptable salt thereof for use as in claim 129, wherein the reduction or elimination of negative thinking as reflected by an improvement in the score of the MADRS item pessimistic thoughts persists until at least 14 days after the last administration of 5-MeO-DMT or a pharmaceutically acceptable salt thereof.

132. 5-MeO-DMT or a pharmaceutically acceptable salt thereof for use as in claim 129, wherein the reduction or elimination of negative thinking as reflected by an improvement in the score of the MADRS item pessimistic thoughts persists until at least 28 days after the last administration of 5-MeO-DMT or a pharmaceutically acceptable salt thereof.

133. 5-MeO-DMT or a pharmaceutically acceptable salt thereof for use as in claim 113 or 114, wherein the reduction or elimination of negative thinking is reflected by an improvement at least in the score of the BPRS item guilt feelings about 2 hours after the last administration of 5-MeO-DMT or a pharmaceutically acceptable salt thereof.

134. 5-MeO-DMT or a pharmaceutically acceptable salt thereof for use as in claim 113, 114 or 133, wherein the reduction or elimination of negative thinking is reflected by an improvement at least in the score of the BPRS item guilt feelings on day 1, for instance, about 24 hours after the last administration of 5-MeO-DMT or a pharmaceutically acceptable salt thereof.

135. 5-MeO-DMT or a pharmaceutically acceptable salt thereof for use as in claim 113, 114, 133 or 134, wherein the reduction or elimination of negative thinking is reflected by an improvement at least in the score of the BPRS item guilt feelings on day 7 after the last administration of 5-MeO-DMT or a pharmaceutically acceptable salt thereof.

136. 5-MeO-DMT or a pharmaceutically acceptable salt thereof for use as in claims 113, 114 or 133 to 135, wherein the reduction or elimination of negative thinking is reflected by an improvement at least in the score of the BPRS item guilt feelings on day 14 after the last administration of 5-MeO-DMT or a pharmaceutically acceptable salt thereof.

137. 5-MeO-DMT or a pharmaceutically acceptable salt thereof for use as in claims 113, 114 or 133 to 136 wherein the reduction or elimination of negative thinking is reflected by an improvement at least in the score of the BPRS item guilt feelings on day 28 after the last administration of 5-MeO-DMT or a pharmaceutically acceptable salt thereof.

138. 5-MeO-DMT or a pharmaceutically acceptable salt thereof for use as in claim 113 or 114, wherein the reduction or elimination of negative thinking as reflected by an improvement in the score of the BPRS item guilt feelings occurs not later than about 2 hours after the last administration of 5-MeO-DMT or a pharmaceutically acceptable salt thereof.

139. 5-MeO-DMT or a pharmaceutically acceptable salt thereof for use as in claim 138, wherein the reduction or elimination of negative thinking as reflected by an improvement in the score of the BPRS item guilt feelings persists until at least 6 days after the last administration of 5-MeO-DMT or a pharmaceutically acceptable salt thereof.

140. 5-MeO-DMT or a pharmaceutically acceptable salt thereof for use as in claim 138, wherein the reduction or elimination of negative thinking as reflected by an improvement in the score of the BPRS item guilt feelings persists until at least 14 days after the last administration of 5-MeO-DMT or a pharmaceutically acceptable salt thereof.

141. 5-MeO-DMT or a pharmaceutically acceptable salt thereof for use as in claim 138, wherein the reduction or elimination of negative thinking as reflected by an improvement in the score of the BPRS item guilt feelings persists until at least 28 days after the last administration of 5-MeO-DMT or a pharmaceutically acceptable salt thereof.

142. 5-MeO-DMT or a pharmaceutically acceptable salt thereof for use as in claim 113 or 114, wherein the patient suffers from negative thinking and an improvement in negative thinking is reflected by a reduction in the Clinical Global Impression-Severity (CGI-S) score about 2 hours after the last administration of 5-MeO-DMT or a pharmaceutically acceptable salt thereof.

143. 5-MeO-DMT or a pharmaceutically acceptable salt thereof for use as in claim 113, 114 or 142, wherein the patient suffers from negative thinking and an improvement in negative thinking is reflected by a reduction in the Clinical Global Impression-Severity (CGI-S) score on day 1, for instance, about 24 hours after the last administration of 5-MeO-DMT or a pharmaceutically acceptable salt thereof.

144. 5-MeO-DMT or a pharmaceutically acceptable salt thereof for use as in claim 113, 114, 142 or 143, wherein the patient suffers from negative thinking and an improvement in negative thinking is reflected by a reduction in the Clinical Global Impression-Severity (CGI-S) score on day 7 after the last administration of 5-MeO-DMT or a pharmaceutically acceptable salt thereof.

145. 5-MeO-DMT or a pharmaceutically acceptable salt thereof for use as in claims 113, 114 or 142 to 144, wherein the patient suffers from negative thinking and an improvement in negative thinking is reflected by a reduction in the Clinical Global Impression-Severity (CGI-S) score on day 14 after the last administration of 5-MeO-DMT or a pharmaceutically acceptable salt thereof.

146. 5-MeO-DMT or a pharmaceutically acceptable salt thereof for use as in claims 113, 114 or 142 to 145, wherein the patient suffers from negative thinking and an improvement in negative thinking is reflected by a reduction in the Clinical Global Impression-Severity (CGI-S) score on day 28 after the last administration of 5-MeO-DMT or a pharmaceutically acceptable salt thereof.

147. 5-MeO-DMT or a pharmaceutically acceptable salt thereof for use as in claims 1 to 112, wherein the patient suffers from negative thinking and an improvement in negative thinking, as reflected by a reduction in the Clinical Global Impression-Severity (CGI-S) score, occurs not later than about 2 hours after the last administration of 5-MeO-DMT or a pharmaceutically acceptable salt thereof.

148. 5-MeO-DMT or a pharmaceutically acceptable salt thereof for use as in claim 147, wherein the improvement in negative thinking, as reflected by a reduction in the Clinical Global Impression-Severity (CGI-S) score, persists until at least 6 days after the last administration of 5-MeO-DMT or a pharmaceutically acceptable salt thereof.

149. 5-MeO-DMT or a pharmaceutically acceptable salt thereof for use as in claim 147, wherein the improvement in negative thinking, as reflected by a reduction in the Clinical Global Impression-Severity (CGI-S) score, persists until at least 14 days after the last administration of 5-MeO-DMT or a pharmaceutically acceptable salt thereof.

150. 5-MeO-DMT or a pharmaceutically acceptable salt thereof for use as in claim 147, wherein the improvement in negative thinking, as reflected by a reduction in the Clinical Global Impression-Severity (CGI-S) score, persists until at least 28 days after the last administration of 5-MeO-DMT or a pharmaceutically acceptable salt thereof.

151. 5-MeO-DMT or a pharmaceutically acceptable salt thereof for use as in claims 1 to 150, wherein the patient suffers from anxiety and the treatment reduces or eliminates the anxiety.

152. 5-MeO-DMT or a pharmaceutically acceptable salt thereof for use as in claim 151, wherein the reduction or elimination of anxiety is reflected by an improvement at least in the score of the BDRS item anxiety 2 hours after the last administration of 5-MeO-DMT or a pharmaceutically acceptable salt thereof.

153. 5-MeO-DMT or a pharmaceutically acceptable salt thereof for use as in claim 151 or 152, wherein the reduction or elimination of anxiety is reflected by an improvement at least in the score of the BDRS item anxiety on day 1, for instance, about 24 hours after the last administration of 5-MeO-DMT or a pharmaceutically acceptable salt thereof.

154. 5-MeO-DMT or a pharmaceutically acceptable salt thereof for use as in claims 151 to 153, wherein the reduction or elimination of anxiety is reflected by an improvement at least in the score of the BDRS item anxiety on day 7 after the last administration of 5-MeO-DMT or a pharmaceutically acceptable salt thereof.

155. 5-MeO-DMT or a pharmaceutically acceptable salt thereof for use as in claims 151 to 154, wherein the reduction or elimination of anxiety is reflected by an improvement at least in the score of the BDRS item anxiety on day 14 after the last administration of 5-MeO-DMT or a pharmaceutically acceptable salt thereof.

156. 5-MeO-DMT or a pharmaceutically acceptable salt thereof for use as in claims 151 to 155, wherein the reduction or elimination of anxiety is reflected by an improvement at least in the score of the BDRS item anxiety on day 28 after the last administration of 5-MeO-DMT or a pharmaceutically acceptable salt thereof.

157. 5-MeO-DMT or a pharmaceutically acceptable salt thereof for use as in claim 151, wherein the reduction or elimination of anxiety as reflected by an improvement in the score of the BDRS item anxiety occurs not later than about 2 hours after the last administration of 5-MeO-DMT or a pharmaceutically acceptable salt thereof.

158. 5-MeO-DMT or a pharmaceutically acceptable salt thereof for use as in claim 157, wherein the reduction or elimination of anxiety as reflected by an improvement in the score of the BDRS item anxiety persists until at least 6 days after the last administration of 5-MeO-DMT or a pharmaceutically acceptable salt thereof.

159. 5-MeO-DMT or a pharmaceutically acceptable salt thereof for use as in claim 157, wherein the reduction or elimination of anxiety as reflected by an improvement in the score of the BDRS item anxiety persists until at least 14 days after the last administration of 5-MeO-DMT or a pharmaceutically acceptable salt thereof.

160. 5-MeO-DMT or a pharmaceutically acceptable salt thereof for use as in claim 157, wherein the reduction or elimination of anxiety as reflected by an improvement in the score of the BDRS item anxiety persists until at least 28 days after the last administration of 5-MeO-DMT or a pharmaceutically acceptable salt thereof.

161. 5-MeO-DMT or a pharmaceutically acceptable salt thereof for use as in claim 151, wherein the reduction or elimination of anxiety is reflected by an improvement at least in the score of the BPRS item anxiety about 2 hours after the last administration of 5-MeO-DMT or a pharmaceutically acceptable salt thereof.

162. 5-MeO-DMT or a pharmaceutically acceptable salt thereof for use as in claim 151 or 161, wherein the reduction or elimination of anxiety is reflected by an improvement at least in the score of the BPRS item anxiety on day 1 after the last administration of 5-MeO-DMT or a pharmaceutically acceptable salt thereof.

163. 5-MeO-DMT or a pharmaceutically acceptable salt thereof for use as in claim 151, 161 or 162, wherein the reduction or elimination of anxiety is reflected by an improvement at least in the score of the BPRS item anxiety on day 7 after the last administration of 5-MeO-DMT or a pharmaceutically acceptable salt thereof.

164. 5-MeO-DMT or a pharmaceutically acceptable salt thereof for use as in claim 151 or 161 to 163, wherein the reduction or elimination of anxiety is reflected by an improvement at least in the score of the BPRS item anxiety on day 14 after the last administration of 5-MeO-DMT or a pharmaceutically acceptable salt thereof.

165. 5-MeO-DMT or a pharmaceutically acceptable salt thereof for use as in claim 151 or 161 to 164, wherein the reduction or elimination of anxiety is reflected by an improvement at least in the score of the BPRS item anxiety on day 28 after the last administration of 5-MeO-DMT or a pharmaceutically acceptable salt thereof.

166. 5-MeO-DMT or a pharmaceutically acceptable salt thereof for use as in claim 151, wherein the reduction or elimination of anxiety as reflected by an improvement in the score of the BPRS item anxiety occurs not later than about 2 hours after the last administration of 5-MeO-DMT or a pharmaceutically acceptable salt thereof.

167. 5-MeO-DMT or a pharmaceutically acceptable salt thereof for use as in claim 166, wherein the reduction or elimination of anxiety as reflected by an improvement in the score of the BPRS item anxiety persists until at least 6 days after the last administration of 5-MeO-DMT or a pharmaceutically acceptable salt thereof.

168. 5-MeO-DMT or a pharmaceutically acceptable salt thereof for use as in claim 166, wherein the reduction or elimination of anxiety as reflected by an improvement in the score of the BPRS item anxiety persists until at least 14 days after the last administration of 5-MeO-DMT or a pharmaceutically acceptable salt thereof.

169. 5-MeO-DMT or a pharmaceutically acceptable salt thereof for use as in claim 166, wherein the reduction or elimination of anxiety as reflected by an improvement in the score of the BPRS item anxiety persists until at least 28 days after the last administration of 5-MeO-DMT or a pharmaceutically acceptable salt thereof.

170. 5-MeO-DMT or a pharmaceutically acceptable salt thereof for use as in claim 151, wherein the patient suffers from anxiety and an improvement in anxiety is reflected by a reduction in the Clinical Global Impression-Severity (CGI-S) score about 2 hours after the last administration of 5-MeO-DMT or a pharmaceutically acceptable salt thereof.

171. 5-MeO-DMT or a pharmaceutically acceptable salt thereof for use as in claim 151 or 170, wherein the patient suffers from anxiety and an improvement in anxiety is reflected by a reduction in the Clinical Global Impression-Severity (CGI-S) score on day 1, for instance, about 24 hours after the last administration of 5-MeO-DMT or a pharmaceutically acceptable salt thereof.

172. 5-MeO-DMT or a pharmaceutically acceptable salt thereof for use as in claim 151, 170 or 171, wherein the patient suffers from anxiety and an improvement in anxiety is reflected by a reduction in the Clinical Global Impression-Severity (CGI-S) score on day 7 after the last administration of 5-MeO-DMT or a pharmaceutically acceptable salt thereof.

173. 5-MeO-DMT or a pharmaceutically acceptable salt thereof for use as in claim 151 or 170 to 172, wherein the patient suffers from anxiety and an improvement in anxiety is reflected by a reduction in the Clinical Global Impression-Severity (CGI-S) score on day 14 after the last administration of 5-MeO-DMT or a pharmaceutically acceptable salt thereof.

174. 5-MeO-DMT or a pharmaceutically acceptable salt thereof for use as in claim 151 or 170 to 173, wherein the patient suffers from anxiety and an improvement in anxiety is reflected by a reduction in the Clinical Global Impression-Severity (CGI-S) score on day 28 after the last administration of 5-MeO-DMT or a pharmaceutically acceptable salt thereof.

175. 5-MeO-DMT or a pharmaceutically acceptable salt thereof for use as in claims 1 to 150, wherein the patient suffers from anxiety and an improvement in anxiety, as reflected by a reduction in the Clinical Global Impression-Severity (CGI-S) score, occurs not later than about 2 hours after the last administration of 5-MeO-DMT or a pharmaceutically acceptable salt thereof.

176. 5-MeO-DMT or a pharmaceutically acceptable salt thereof for use as in claim 175, wherein the improvement in anxiety, as reflected by a reduction in the Clinical Global Impression-Severity (CGI-S) score, persists until at least 6 days after the last administration of 5-MeO-DMT or a pharmaceutically acceptable salt thereof.

177. 5-MeO-DMT or a pharmaceutically acceptable salt thereof for use as in claim 175, wherein the improvement in anxiety, as reflected by a reduction in the Clinical Global Impression-Severity (CGI-S) score, persists until at least 14 days after the last administration of 5-MeO-DMT or a pharmaceutically acceptable salt thereof.

178. 5-MeO-DMT or a pharmaceutically acceptable salt thereof for use as in claim 175, wherein the improvement in anxiety, as reflected by a reduction in the Clinical Global Impression-Severity (CGI-S) score, persists until at least 28 days after the last administration of 5-MeO-DMT or a pharmaceutically acceptable salt thereof.

179. 5-MeO-DMT or a pharmaceutically acceptable salt thereof for use as in claims 1 to 178, wherein the patient suffers from cognitive dysfunction and the treatment reduces or eliminates the cognitive dysfunction.

180. 5-MeO-DMT or a pharmaceutically acceptable salt thereof for use as in claim 179, wherein the reduction or elimination of cognitive dysfunction is reflected by an improvement at least in the score of the BDRS item impaired concentration and memory about 2 hours after the last administration of 5-MeO-DMT or a pharmaceutically acceptable salt thereof.

181. 5-MeO-DMT or a pharmaceutically acceptable salt thereof for use as in claim 179 or 180, wherein the reduction or elimination of cognitive dysfunction is reflected by an improvement at least in the score of the BDRS item impaired concentration and memory on day 1, for instance, about 24 hours after the last administration of 5-MeO-DMT or a the last administration of 5-MeO-DMT or a pharmaceutically acceptable salt thereof.

182. 5-MeO-DMT or a pharmaceutically acceptable salt thereof for use as in claims 179 to 181, wherein the reduction or elimination of cognitive dysfunction is reflected by an improvement at least in the score of the BDRS item impaired concentration and memory on day 7 after the last administration of 5-MeO-DMT or a pharmaceutically acceptable salt thereof.

183. 5-MeO-DMT or a pharmaceutically acceptable salt thereof for use as in claims 179 to 182, wherein the reduction or elimination of cognitive dysfunction is reflected by an improvement at least in the score of the BDRS item impaired concentration and memory on day 14 after the last administration of 5-MeO-DMT or a pharmaceutically acceptable salt thereof.

184. 5-MeO-DMT or a pharmaceutically acceptable salt thereof for use as in claims 179 to 183, wherein the reduction or elimination of cognitive dysfunction is reflected by an improvement at least in the score of the BDRS item impaired concentration and memory on day 28 after the last administration of 5-MeO-DMT or a pharmaceutically acceptable salt thereof.

185. 5-MeO-DMT or a pharmaceutically acceptable salt thereof for use as in claim 179, wherein the reduction or elimination of cognitive dysfunction as reflected by an improvement in the score of the BDRS item impaired concentration and memory occurs not later than about 2 hours after the last administration of 5-MeO-DMT or a pharmaceutically acceptable salt thereof.

186. 5-MeO-DMT or a pharmaceutically acceptable salt thereof for use as in claim 185, wherein the reduction or elimination of cognitive dysfunction as reflected by an improvement in the score of the BDRS item impaired concentration and memory persists until at least 6 days after the last administration of 5-MeO-DMT or a pharmaceutically acceptable salt thereof.

187. 5-MeO-DMT or a pharmaceutically acceptable salt thereof for use as in claim 185, wherein the reduction or elimination of cognitive dysfunction as reflected by an improvement in the score of the BDRS item impaired concentration and memory persists until at least 14 days after the last administration of 5-MeO-DMT or a pharmaceutically acceptable salt thereof.

188. 5-MeO-DMT or a pharmaceutically acceptable salt thereof for use as in claim 185, wherein the reduction or elimination of cognitive dysfunction as reflected by an improvement in the score of the BDRS item impaired concentration and memory persists until at least 28 days after the last administration of 5-MeO-DMT or a pharmaceutically acceptable salt thereof.

189. 5-MeO-DMT or a pharmaceutically acceptable salt thereof for use as in claim 179, wherein the reduction or elimination of cognitive dysfunction is reflected by an improvement at least in the score of the MADRS item concentration difficulties about 2 hours after the last administration of 5-MeO-DMT or a pharmaceutically acceptable salt thereof.

190. 5-MeO-DMT or a pharmaceutically acceptable salt thereof for use as in claim 179 or 189, wherein the reduction or elimination of cognitive dysfunction is reflected by an improvement at least in the score of the MADRS item concentration difficulties on day 1, for instance, about 24 hours after the last administration of 5-MeO-DMT or a pharmaceutically acceptable salt thereof.

191. 5-MeO-DMT or a pharmaceutically acceptable salt thereof for use as in claim 179, 189 or 190, wherein the reduction or elimination of cognitive dysfunction is reflected by an improvement at least in the score of the MADRS item concentration difficulties on day 7 after the last administration of 5-MeO-DMT or a pharmaceutically acceptable salt thereof.

192. 5-MeO-DMT or a pharmaceutically acceptable salt thereof for use as in claim 179 or 189 to 191, wherein the reduction or elimination of cognitive dysfunction is reflected by an improvement at least in the score of the MADRS item concentration difficulties on day 14 after the last administration of 5-MeO-DMT or a pharmaceutically acceptable salt thereof.

193. 5-MeO-DMT or a pharmaceutically acceptable salt thereof for use as in claim 179 or 189 to 192, wherein the reduction or elimination of cognitive dysfunction is reflected by an improvement at least in the score of the MADRS item concentration difficulties on day 28 after the last administration of 5-MeO-DMT or a pharmaceutically acceptable salt thereof.

194. 5-MeO-DMT or a pharmaceutically acceptable salt thereof for use as in claim 179, wherein the reduction or elimination of cognitive dysfunction as reflected by an improvement in the score of the MADRS item concentration difficulties occurs not later than about 2 hours after the last administration of 5-MeO-DMT or a pharmaceutically acceptable salt thereof.

195. 5-MeO-DMT or a pharmaceutically acceptable salt thereof for use as in claim 194, wherein the reduction or elimination of cognitive dysfunction as reflected by an improvement in the score of the MADRS item concentration difficulties persists until at least 6 days after the last administration of 5-MeO-DMT or a pharmaceutically acceptable salt thereof.

196. 5-MeO-DMT or a pharmaceutically acceptable salt thereof for use as in claim 194, wherein the reduction or elimination of cognitive dysfunction as reflected by an improvement in the score of the MADRS item concentration difficulties persists until at least 14 days after the last administration of 5-MeO-DMT or a pharmaceutically acceptable salt thereof.

197. 5-MeO-DMT or a pharmaceutically acceptable salt thereof for use as in claim 194, wherein the reduction or elimination of cognitive dysfunction as reflected by an improvement in the score of the MADRS item concentration difficulties persists until at least 28 days after the last administration of 5-MeO-DMT or a pharmaceutically acceptable salt thereof.

198. 5-MeO-DMT or a pharmaceutically acceptable salt thereof for use as in claim 179, wherein the patient suffers from cognitive dysfunction and an improvement in cognitive dysfunction is reflected by a reduction in the Clinical Global Impression-Severity (CGI-S) score about 2 hours after the last administration of 5-MeO-DMT or a pharmaceutically acceptable salt thereof.

199. 5-MeO-DMT or a pharmaceutically acceptable salt thereof for use as in claim 179 or 198, wherein the patient suffers from cognitive dysfunction and an improvement in cognitive dysfunction is reflected by a reduction in the Clinical Global Impression-Severity (CGI-S) score on day 1, for instance, about 24 hours after the last administration of 5-MeO-DMT or a pharmaceutically acceptable salt thereof.

200. 5-MeO-DMT or a pharmaceutically acceptable salt thereof for use as in claim 179, 198 or 199, wherein the patient suffers from cognitive dysfunction and an improvement in cognitive dysfunction is reflected by a reduction in the Clinical Global Impression-Severity (CGI-S) score on day 7 after the last administration of 5-MeO-DMT or a pharmaceutically acceptable salt thereof.

201. 5-MeO-DMT or a pharmaceutically acceptable salt thereof for use as in claim 179 or 198 to 200, wherein the patient suffers from cognitive dysfunction and an improvement in cognitive dysfunction is reflected by a reduction in the Clinical Global Impression-Severity (CGI-S) score on day 14 after the last administration of 5-MeO-DMT or a pharmaceutically acceptable salt thereof.

202. 5-MeO-DMT or a pharmaceutically acceptable salt thereof for use as in claim 179 or 198 to 201, wherein the patient suffers from cognitive dysfunction and an improvement in cognitive dysfunction is reflected by a reduction in the Clinical Global Impression-Severity (CGI-S) score on day 28 after the last administration of 5-MeO-DMT or a pharmaceutically acceptable salt thereof.

203. 5-MeO-DMT or a pharmaceutically acceptable salt thereof for use as in claims 1 to 178, wherein the patient suffers from cognitive dysfunction and an improvement in cognitive dysfunction, as reflected by a reduction in the Clinical Global Impression-Severity (CGI-S) score, occurs not later than about 2 hours after the last administration of 5-MeO-DMT or a pharmaceutically acceptable salt thereof.

204. 5-MeO-DMT or a pharmaceutically acceptable salt thereof for use as in claim 203, wherein the improvement in cognitive dysfunction, as reflected by a reduction in the Clinical Global Impression-Severity (CGI-S) score, persists until at least 6 days after the last administration of 5-MeO-DMT or a pharmaceutically acceptable salt thereof.

205. 5-MeO-DMT or a pharmaceutically acceptable salt thereof for use as in claim 203, wherein the improvement in cognitive dysfunction, as reflected by a reduction in the Clinical Global Impression-Severity (CGI-S) score, persists until at least 14 days after the last administration of 5-MeO-DMT or a pharmaceutically acceptable salt thereof.

206. 5-MeO-DMT or a pharmaceutically acceptable salt thereof for use as in claim 203, wherein the improvement in cognitive dysfunction, as reflected by a reduction in the Clinical Global Impression-Severity (CGI-S) score, persists until at least 28 days after the last administration of 5-MeO-DMT or a pharmaceutically acceptable salt thereof.

207. 5-MeO-DMT or a pharmaceutically acceptable salt thereof for use as in claims 1 to 206, wherein the patient suffers from social/emotional withdrawal or detachment and the treatment reduces or eliminates the social/emotional withdrawal or detachment.

208. 5-MeO-DMT or a pharmaceutically acceptable salt thereof for use as in claim 207, wherein the treatment reduces or eliminates at least one of anhedonia, emotional withdrawal and affective flattening.

209. 5-MeO-DMT or a pharmaceutically acceptable salt thereof for use as in claim 207 or 208, wherein the reduction or elimination of social/emotional withdrawal or detachment is reflected by an improvement at least in the scores of the BDRS items anhedonia, emotional withdrawal and/or affective flattening about 2 hours after the last administration of 5-MeO-DMT or a pharmaceutically acceptable salt thereof.

210. 5-MeO-DMT or a pharmaceutically acceptable salt thereof for use as in claims 207 to 209, wherein the reduction or elimination of social/emotional withdrawal or detachment is reflected by an improvement at least in the scores of the BDRS items anhedonia, emotional withdrawal and/or affective flattening on day 1, for instance, about 24 hours after the last administration of 5-MeO-DMT or a pharmaceutically acceptable salt thereof.

211. 5-MeO-DMT or a pharmaceutically acceptable salt thereof for use as in claims 207 to 210, wherein the reduction or elimination of social/emotional withdrawal or detachment is reflected by an improvement at least in the score of the BDRS items anhedonia, emotional withdrawal and/or affective flattening on day 7 after the last administration of 5-MeO-DMT or a pharmaceutically acceptable salt thereof.

212. 5-MeO-DMT or a pharmaceutically acceptable salt thereof for use as in claims 207 to 211, wherein the reduction or elimination of social/emotional withdrawal or detachment is reflected by an improvement at least in the scores of the BDRS items anhedonia, emotional withdrawal and/or affective flattening on day 14 after the last administration of 5-MeO-DMT or a pharmaceutically acceptable salt thereof.

213. 5-MeO-DMT or a pharmaceutically acceptable salt thereof for use as in claims 207 to 212, wherein the reduction or elimination of social/emotional withdrawal or detachment is reflected by an improvement at least in the scores of the BDRS items anhedonia, emotional withdrawal and/or affective flattening on day 28 after the last administration of 5-MeO-DMT or a pharmaceutically acceptable salt thereof.

214. 5-MeO-DMT or a pharmaceutically acceptable salt thereof for use as in claim 207 or 208, wherein the reduction or elimination of social/emotional withdrawal or detachment as reflected by an improvement in the scores of the BDRS items anhedonia, emotional withdrawal and/or affective flattening occurs not later than about 2 hours after the last administration of 5-MeO-DMT or a pharmaceutically acceptable salt thereof.

215. 5-MeO-DMT or a pharmaceutically acceptable salt thereof for use as in claim 214, wherein the reduction or elimination of social/emotional withdrawal or detachment as reflected by an improvement in the scores of the BDRS items anhedonia, emotional withdrawal and/or affective flattening persists until at least 6 days after the last administration of 5-MeO-DMT or a pharmaceutically acceptable salt thereof.

216. 5-MeO-DMT or a pharmaceutically acceptable salt thereof for use as in claim 214, wherein the reduction or elimination of social/emotional withdrawal or detachment as reflected by an improvement in the scores of the BDRS items anhedonia, emotional withdrawal and/or affective flattening persists until at least 14 days after the last administration of 5-MeO-DMT or a pharmaceutically acceptable salt thereof.

217. 5-MeO-DMT or a pharmaceutically acceptable salt thereof for use as in claim 214, wherein the reduction or elimination of social/emotional withdrawal or detachment as reflected by an improvement in the scores of the BDRS items anhedonia, emotional withdrawal and/or affective flattening persists until at least 28 days after the last administration of 5-MeO-DMT or a pharmaceutically acceptable salt thereof.

218. 5-MeO-DMT or a pharmaceutically acceptable salt thereof for use as in claim 207 or 208, wherein the reduction or elimination of social/emotional withdrawal or detachment is reflected by an improvement at least in the score of the MADRS item inability to feel about 2 hours after the last administration of 5-MeO-DMT or a pharmaceutically acceptable salt thereof.

219. 5-MeO-DMT or a pharmaceutically acceptable salt thereof for use as in claim 207, 208 or 218, wherein the reduction or elimination of social/emotional withdrawal or detachment is reflected by an improvement at least in the score of the MADRS item inability to feel on day 1, for instance, about 24 hours days after the last administration of 5-MeO-DMT or a pharmaceutically acceptable salt thereof.

220. 5-MeO-DMT or a pharmaceutically acceptable salt thereof for use as in claim 207, 208, 218 or 219, wherein the reduction or elimination of social/emotional withdrawal or detachment is reflected by an improvement at least in the score of the MADRS item inability to feel on day 7 days after the last administration of 5-MeO-DMT or a pharmaceutically acceptable salt thereof.

221. 5-MeO-DMT or a pharmaceutically acceptable salt thereof for use as in claims 207, 208 or 218 to 220, wherein the reduction or elimination of social/emotional withdrawal or detachment is reflected by an improvement at least in the score of the MADRS item inability to feel on day 14 days after the last administration of 5-MeO-DMT or a pharmaceutically acceptable salt thereof.

222. 5-MeO-DMT or a pharmaceutically acceptable salt thereof for use as in claims 207, 208 or 218 to 221, wherein the reduction or elimination of social/emotional withdrawal or detachment is reflected by an improvement at least in the score of the MADRS item inability to feel on day 28 after the last administration of 5-MeO-DMT or a pharmaceutically acceptable salt thereof.

223. 5-MeO-DMT or a pharmaceutically acceptable salt thereof for use as in claim 207 or 208, wherein the reduction or elimination of social/emotional withdrawal or detachment as reflected by an improvement in the score of the MADRS item inability to feel occurs not later than about 2 hours after the last administration of 5-MeO-DMT or a pharmaceutically acceptable salt thereof.

224. 5-MeO-DMT or a pharmaceutically acceptable salt thereof for use as in claim 223, wherein the reduction or elimination of social/emotional withdrawal or detachment as reflected by an improvement in the score of the MADRS item inability to feel persists until at least 6 days after the last administration of 5-MeO-DMT or a pharmaceutically acceptable salt thereof.

225. 5-MeO-DMT or a pharmaceutically acceptable salt thereof for use as in claim 223, wherein the reduction or elimination of social/emotional withdrawal or detachment as reflected by an improvement in the score of the MADRS item inability to feel persists until at least 14 days after the last administration of 5-MeO-DMT or a pharmaceutically acceptable salt thereof.

226. 5-MeO-DMT or a pharmaceutically acceptable salt thereof for use as in claim 223, wherein the reduction or elimination of social/emotional withdrawal or detachment as reflected by an improvement in the score of the MADRS item inability to feel persists until at least 28 days after the last administration of 5-MeO-DMT or a pharmaceutically acceptable salt thereof.

227. 5-MeO-DMT or a pharmaceutically acceptable salt thereof for use as in claim 207 or 208, wherein the reduction or elimination of social/emotional withdrawal or detachment is reflected by an improvement at least in the score of the BPRS item emotional withdrawal about 2 hours after the last administration of 5-MeO-DMT or a pharmaceutically acceptable salt thereof.

228. 5-MeO-DMT or a pharmaceutically acceptable salt thereof for use as in claim 207, 208 or 227, wherein the reduction or elimination of social/emotional withdrawal or detachment is reflected by an improvement at least in the score of the BPRS item emotional withdrawal on day 1, for instance, about 24 hours after the last administration of 5-MeO-DMT or a pharmaceutically acceptable salt thereof.

229. 5-MeO-DMT or a pharmaceutically acceptable salt thereof for use as in claim 207, 208, 227 or 228, wherein the reduction or elimination of social/emotional withdrawal or detachment is reflected by an improvement at least in the score of the BPRS item emotional withdrawal on day 7 after the last administration of 5-MeO-DMT or a pharmaceutically acceptable salt thereof.

230. 5-MeO-DMT or a pharmaceutically acceptable salt thereof for use as in claims 207, 208 or 227 to 229, wherein the reduction or elimination of social/emotional withdrawal or detachment is reflected by an improvement at least in the score of the BPRS item emotional withdrawal on day 14 after the last administration of 5-MeO-DMT or a pharmaceutically acceptable salt thereof.

231. 5-MeO-DMT or a pharmaceutically acceptable salt thereof for use as in claims 207, 208 or 227 to 230, wherein the reduction or elimination of social/emotional withdrawal or detachment is reflected by an improvement at least in the score of the BPRS item emotional withdrawal on day 28 after the last administration of 5-MeO-DMT or a pharmaceutically acceptable salt thereof.

232. 5-MeO-DMT or a pharmaceutically acceptable salt thereof for use as in claim 207 or 208, wherein the reduction or elimination of social/emotional withdrawal or detachment as reflected by an improvement in the score of the BPRS item emotional withdrawal occurs not later than about 2 hours after the last administration of 5-MeO-DMT or a pharmaceutically acceptable salt thereof.

233. 5-MeO-DMT or a pharmaceutically acceptable salt thereof for use as in claim 232, wherein the reduction or elimination of social/emotional withdrawal or detachment as reflected by an improvement in the score of the BPRS item emotional withdrawal persists until at least 6 days after the last administration of 5-MeO-DMT or a pharmaceutically acceptable salt thereof.

234. 5-MeO-DMT or a pharmaceutically acceptable salt thereof for use as in claim 232, wherein the reduction or elimination of social/emotional withdrawal or detachment as reflected by an improvement in the score of the BPRS item emotional withdrawal persists until at least 14 days after the last administration of 5-MeO-DMT or a pharmaceutically acceptable salt thereof.

235. 5-MeO-DMT or a pharmaceutically acceptable salt thereof for use as in claim 232, wherein the reduction or elimination of social/emotional withdrawal or detachment as reflected by an improvement in the score of the BPRS item emotional withdrawal persists until at least 28 days after the last administration of 5-MeO-DMT or a pharmaceutically acceptable salt thereof.

236. 5-MeO-DMT or a pharmaceutically acceptable salt thereof for use as in claim 207 or 208, wherein the reduction or elimination of social/emotional withdrawal or detachment is reflected by an improvement at least in the score of the BPRS item blunted affect about 2 hours after the last administration of 5-MeO-DMT or a pharmaceutically acceptable salt thereof.

237. 5-MeO-DMT or a pharmaceutically acceptable salt thereof for use as in claim 207, 208 or 236, wherein the reduction or elimination of social/emotional withdrawal or detachment is reflected by an improvement at least in the score of the BPRS item blunted affect on day 1, for instance, about 24 hours after the last administration of 5-MeO-DMT or a pharmaceutically acceptable salt thereof.

238. 5-MeO-DMT or a pharmaceutically acceptable salt thereof for use as in claim 207, 208, 236 or 237, wherein the reduction or elimination of social/emotional withdrawal or detachment is reflected by an improvement at least in the score of the BPRS item blunted affect on day 7 after the last administration of 5-MeO-DMT or a pharmaceutically acceptable salt thereof.

239. 5-MeO-DMT or a pharmaceutically acceptable salt thereof for use as in claims 207, 208 or 236 to 238, wherein the reduction or elimination of social/emotional withdrawal or detachment is reflected by an improvement at least in the score of the BPRS item blunted affect on day 14 after the last administration of 5-MeO-DMT or a pharmaceutically acceptable salt thereof.

240. 5-MeO-DMT or a pharmaceutically acceptable salt thereof for use as in claims 207, 208 or 236 to 239, wherein the reduction or elimination of social/emotional withdrawal or detachment is reflected by an improvement at least in the score of the BPRS item blunted affect on day 28 after the last administration of 5-MeO-DMT or a pharmaceutically acceptable salt thereof.

241. 5-MeO-DMT or a pharmaceutically acceptable salt thereof for use as in claim 207 or 208, wherein the reduction or elimination of social/emotional withdrawal or detachment as reflected by an improvement in the score of the BPRS item blunted affect occurs not later than about 2 hours after the last administration of 5-MeO-DMT or a pharmaceutically acceptable salt thereof.

242. 5-MeO-DMT or a pharmaceutically acceptable salt thereof for use as in claim 241, wherein the reduction or elimination of social/emotional withdrawal or detachment as reflected by an improvement in the score of the BPRS item blunted affect persists until at least 6 days after the last administration of 5-MeO-DMT or a pharmaceutically acceptable salt thereof.

243. 5-MeO-DMT or a pharmaceutically acceptable salt thereof for use as in claim 241, wherein the reduction or elimination of social/emotional withdrawal or detachment as reflected by an improvement in the score of the BPRS item blunted affect persists until at least 14 days after the last administration of 5-MeO-DMT or a pharmaceutically acceptable salt thereof.

244. 5-MeO-DMT or a pharmaceutically acceptable salt thereof for use as in claim 241, wherein the reduction or elimination of social/emotional withdrawal or detachment as reflected by an improvement in the score of the BPRS item blunted affect persists until at least 28 days after the last administration of 5-MeO-DMT or a pharmaceutically acceptable salt thereof.

245. 5-MeO-DMT or a pharmaceutically acceptable salt thereof for use as in claim 207 or 208, wherein the patient suffers from social/emotional withdrawal or detachment and an improvement in social/emotional withdrawal or detachment is reflected by a reduction in the Clinical Global Impression-Severity (CGI-S) score about 2 hours after the last administration of 5-MeO-DMT or a pharmaceutically acceptable salt thereof.

246. 5-MeO-DMT or a pharmaceutically acceptable salt thereof for use as in claim 207, 208 or 245, wherein the patient suffers from social/emotional withdrawal or detachment and an improvement in social/emotional withdrawal or detachment is reflected by a reduction in the Clinical Global Impression-Severity (CGI-S) score on day 1, for instance, about 24 hours after the last administration of 5-MeO-DMT or a pharmaceutically acceptable salt thereof.

247. 5-MeO-DMT or a pharmaceutically acceptable salt thereof for use as in claim 207, 208, 245 or 246, wherein the patient suffers from social/emotional withdrawal or detachment and an improvement in social/emotional withdrawal or detachment is reflected by a reduction in the Clinical Global Impression-Severity (CGI-S) score on day 7 after the last administration of 5-MeO-DMT or a pharmaceutically acceptable salt thereof.

248. 5-MeO-DMT or a pharmaceutically acceptable salt thereof for use as in claims 207, 208 or 245 to 247, wherein the patient suffers from social/emotional withdrawal or detachment and an improvement in social/emotional withdrawal or detachment is reflected by a reduction in the Clinical Global Impression-Severity (CGI-S) score on day 14 after the last administration of 5-MeO-DMT or a pharmaceutically acceptable salt thereof.

249. 5-MeO-DMT or a pharmaceutically acceptable salt thereof for use as in claims 207, 208 or 245 to 248, wherein the patient suffers from social/emotional withdrawal or detachment and an improvement in social/emotional withdrawal or detachment is reflected by a reduction in the Clinical Global Impression-Severity (CGI-S) score on day 28 after the last administration of 5-MeO-DMT or a pharmaceutically acceptable salt thereof.

250. 5-MeO-DMT or a pharmaceutically acceptable salt thereof for use as in claims 1 to 206, wherein the patient suffers from social/emotional withdrawal or detachment and an improvement at least in social/emotional withdrawal or detachment, as reflected by a reduction in the Clinical Global Impression-Severity (CGI-S) score, occurs not later than about 2 hours after the last administration of 5-MeO-DMT or a pharmaceutically acceptable salt thereof.

251. 5-MeO-DMT or a pharmaceutically acceptable salt thereof for use as in claim 250, wherein the improvement in social/emotional withdrawal or detachment, as reflected by a reduction at least in the Clinical Global Impression-Severity (CGI-S) score, persists until at least 6 days after the last administration of 5-MeO-DMT or a pharmaceutically acceptable salt thereof.

252. 5-MeO-DMT or a pharmaceutically acceptable salt thereof for use as in claim 251, wherein the improvement in social/emotional withdrawal or detachment, as reflected by a reduction at least in the Clinical Global Impression-Severity (CGI-S) score, persists until at least 14 days after the last administration of 5-MeO-DMT or a pharmaceutically acceptable salt thereof.

253. 5-MeO-DMT or a pharmaceutically acceptable salt thereof for use as in claim 251, wherein the improvement in social/emotional withdrawal or detachment, as reflected by a reduction at least in the Clinical Global Impression-Severity (CGI-S) score, persists until at least 28 days after the last administration of 5-MeO-DMT or a pharmaceutically acceptable salt thereof.

254. 5-MeO-DMT or a pharmaceutically acceptable salt thereof for use as in claims 1 to 253, wherein the treatment reduces or eliminates suicidal ideation.

255. 5-MeO-DMT or a pharmaceutically acceptable salt thereof for use as in claim 254, wherein the reduction or elimination of suicidal ideation is reflected by an improvement at least in the score of the BDRS item suicidal ideation about 2 hours after the last administration of 5-MeO-DMT or a pharmaceutically acceptable salt thereof.

256. 5-MeO-DMT or a pharmaceutically acceptable salt thereof for use as in claim 254 or 255, wherein the reduction or elimination of suicidal ideation is reflected by an improvement at least in the score of the BDRS item suicidal ideation on day 1, for instance, about 24 hours after the last administration of 5-MeO-DMT or a pharmaceutically acceptable salt thereof.

257. 5-MeO-DMT or a pharmaceutically acceptable salt thereof for use as in claims 254 to 256, wherein the reduction or elimination of suicidal ideation is reflected by an improvement at least in the score of the BDRS item suicidal ideation on day 7 after the last administration of 5-MeO-DMT or a pharmaceutically acceptable salt thereof.

258. 5-MeO-DMT or a pharmaceutically acceptable salt thereof for use as in claims 254 to 257, wherein the reduction or elimination of suicidal ideation is reflected by an improvement at least in the score of the BDRS item suicidal ideation on day 14 after the last administration of 5-MeO-DMT or a pharmaceutically acceptable salt thereof.

259. 5-MeO-DMT or a pharmaceutically acceptable salt thereof for use as in claims 254 to 258, wherein the reduction or elimination of suicidal ideation is reflected by an improvement at least in the score of the BDRS item suicidal ideation on day 28 after the last administration of 5-MeO-DMT or a pharmaceutically acceptable salt thereof.

260. 5-MeO-DMT or a pharmaceutically acceptable salt thereof for use as in claim 254, wherein the reduction or elimination of suicidal ideation as reflected by an improvement in the score of the BDRS item suicidal ideation occurs not later than about 2 hours after the last administration of 5-MeO-DMT or a pharmaceutically acceptable salt thereof.

261. 5-MeO-DMT or a pharmaceutically acceptable salt thereof for use as in claim 260, wherein the reduction or elimination of suicidal ideation as reflected by an improvement in the score of the BDRS item suicidal ideation persists until at least 6 days after the last administration of 5-MeO-DMT or a pharmaceutically acceptable salt thereof.

262. 5-MeO-DMT or a pharmaceutically acceptable salt thereof for use as in claim 260, wherein the reduction or elimination of suicidal ideation as reflected by an improvement in the score of the BDRS item suicidal ideation persists until at least 14 days after the last administration of 5-MeO-DMT or a pharmaceutically acceptable salt thereof.

263. 5-MeO-DMT or a pharmaceutically acceptable salt thereof for use as in claim 260, wherein the reduction or elimination of suicidal ideation as reflected by an improvement in the score of the BDRS item suicidal ideation persists until at least 28 days after the last administration of 5-MeO-DMT or a pharmaceutically acceptable salt thereof.

264. 5-MeO-DMT or a pharmaceutically acceptable salt thereof for use as in claim 254, wherein the reduction or elimination of suicidal ideation is reflected by an improvement at least in the score of the MADRS item suicidal thoughts about 2 hours after the last administration of 5-MeO-DMT or a pharmaceutically acceptable salt thereof.

265. 5-MeO-DMT or a pharmaceutically acceptable salt thereof for use as in claim 254 or 264, wherein the reduction or elimination of suicidal ideation is reflected by an improvement at least in the score of the MADRS item suicidal thoughts on day 1, for instance, about 24 hours after the last administration of 5-MeO-DMT or a pharmaceutically acceptable salt thereof.

266. 5-MeO-DMT or a pharmaceutically acceptable salt thereof for use as in claim 254, 264 or 265, wherein the reduction or elimination of suicidal ideation is reflected by an improvement at least in the score of the MADRS item suicidal thoughts on day 7 after the last administration of 5-MeO-DMT or a pharmaceutically acceptable salt thereof.

267. 5-MeO-DMT or a pharmaceutically acceptable salt thereof for use as in claim 254 or 264 to 266, wherein the reduction or elimination of suicidal ideation is reflected by an improvement at least in the score of the MADRS item suicidal thoughts on day 14 after the last administration of 5-MeO-DMT or a pharmaceutically acceptable salt thereof.

268. 5-MeO-DMT or a pharmaceutically acceptable salt thereof for use as in claim 254 or 264 to 267, wherein the reduction or elimination of suicidal ideation is reflected by an improvement at least in the score of the MADRS item suicidal thoughts on day 28 after the last administration of 5-MeO-DMT or a pharmaceutically acceptable salt thereof.

269. 5-MeO-DMT or a pharmaceutically acceptable salt thereof for use as in claim 254, wherein the reduction or elimination of suicidal ideation as reflected by an improvement in the score of the MADRS item suicidal thoughts occurs not later than about 2 hours after the last administration of 5-MeO-DMT or a pharmaceutically acceptable salt thereof.

270. 5-MeO-DMT or a pharmaceutically acceptable salt thereof for use as in claim 269, wherein the reduction or elimination of suicidal ideation as reflected by an improvement in the score of the MADRS item suicidal thoughts persists until at least 6 days after the last administration of 5-MeO-DMT or a pharmaceutically acceptable salt thereof.

271. 5-MeO-DMT or a pharmaceutically acceptable salt thereof for use as in claim 269, wherein the reduction or elimination of suicidal ideation as reflected by an improvement in the score of the MADRS item suicidal thoughts persists until at least 14 days after the last administration of 5-MeO-DMT or a pharmaceutically acceptable salt thereof.

272. 5-MeO-DMT or a pharmaceutically acceptable salt thereof for use as in claim 269, wherein the reduction or elimination of suicidal ideation as reflected by an improvement in the score of the MADRS item suicidal thoughts persists until at least 28 days after the last administration of 5-MeO-DMT or a pharmaceutically acceptable salt thereof.

273. 5-MeO-DMT or a pharmaceutically acceptable salt thereof for use as in claim 254, wherein the patient suffers from suicidal ideation and an improvement in suicidal ideation is reflected by a reduction in the Clinical Global Impression-Severity (CGI-S) score about 2 hours after the last administration of 5-MeO-DMT or a pharmaceutically acceptable salt thereof.

274. 5-MeO-DMT or a pharmaceutically acceptable salt thereof for use as in claim 254 or 273, wherein the patient suffers from suicidal ideation and an improvement in suicidal ideation is reflected by a reduction in the Clinical Global Impression-Severity (CGI-S) score on day 1, for instance, about 24 hours after the last administration of 5-MeO-DMT or a pharmaceutically acceptable salt thereof.

275. 5-MeO-DMT or a pharmaceutically acceptable salt thereof for use as in claim 254, 273 or 274, wherein the patient suffers from suicidal ideation and an improvement in suicidal ideation is reflected by a reduction in the Clinical Global Impression-Severity (CGI-S) score on day 7 after the last administration of 5-MeO-DMT or a pharmaceutically acceptable salt thereof.

276. 5-MeO-DMT or a pharmaceutically acceptable salt thereof for use as in claim 254 or 273 to 275, wherein the patient suffers from suicidal ideation and an improvement in suicidal ideation is reflected by a reduction in the Clinical Global Impression-Severity (CGI-S) score on day 14 after the last administration of 5-MeO-DMT or a pharmaceutically acceptable salt thereof.

277. 5-MeO-DMT or a pharmaceutically acceptable salt thereof for use as in claim 254 or 273 to 276, wherein the patient suffers from suicidal ideation and an improvement in suicidal ideation is reflected by a reduction in the Clinical Global Impression-Severity (CGI-S) score on day 28 after the last administration of 5-MeO-DMT or a pharmaceutically acceptable salt thereof.

278. 5-MeO-DMT or a pharmaceutically acceptable salt thereof for use as in claims 1 to 253, wherein the patient suffers from suicidal ideation and an improvement in suicidal ideation, as reflected by a reduction in the Clinical Global Impression-Severity (CGI-S) score, occurs not later than about 2 hours after the last administration of 5-MeO-DMT or a pharmaceutically acceptable salt thereof.

279. 5-MeO-DMT or a pharmaceutically acceptable salt thereof for use as in claim 278, wherein the improvement in suicidal ideation, as reflected by a reduction in the Clinical Global Impression-Severity (CGI-S) score, persists until at least 6 days after the last administration of 5-MeO-DMT or a pharmaceutically acceptable salt thereof.

280. 5-MeO-DMT or a pharmaceutically acceptable salt thereof for use as in claim 278, wherein the improvement in suicidal ideation, as reflected by a reduction in the Clinical Global Impression-Severity (CGI-S) score, persists until at least 14 days after the last administration of 5-MeO-DMT or a pharmaceutically acceptable salt thereof.

281. 5-MeO-DMT or a pharmaceutically acceptable salt thereof for use as in claim 278, wherein the improvement in suicidal ideation, as reflected by a reduction in the Clinical Global Impression-Severity (CGI-S) score, persists until at least 28 days after the last administration of 5-MeO-DMT or a pharmaceutically acceptable salt thereof.

282. 5-MeO-DMT or a pharmaceutically acceptable salt thereof for use as in claims 1 to 281, wherein the treatment reduces or eliminates at least one of psychotic symptoms; irritability; lability; increased motor drive; increased speech; agitation.
